(19) United States
(12) Patent Application Publication
(10) Pub. No.: US 2023/0135885 A1
(43) Pub. Date: May 4, 2023

(54) METHOD AND APPARATUS FOR LOADING AND/OR UNLOADING SUTURE AND/OR A SHUTTLE

(71) Applicant: Maruho Medical, Inc., Reno, NV (US)

(72) Inventors: Brett A. SNYDER, Reno, NV (US); John L. MINCK, JR., Reno, NV (US); Ryan N. SCHMID, Reno, NV (US); Andrew J. HUNTINGTON, Reno, NV (US)

(73) Assignee: Maruho Medical, Inc., Reno, NV (US)

(21) Appl. No.: 17/822,391

(22) Filed: Aug. 25, 2022

Related U.S. Application Data
(60) Provisional application No. 63/260,667, filed on Aug. 27, 2021.

(51) Int. Cl. A61B 17/04

(52) U.S. Cl. CPC A61B 17/0401; A61B 2017/0496; A61B 2017/0406; A61B 2017/0409

(57) ABSTRACT

A device is disclosed that can load a suture and/or a shuttle into a device. The device can have a suture and/or a shuttle that can be moved (e.g., via a loader control) from a non-loaded configuration to a loaded configuration without damaging the suture or the shuttle.

### CROSS-REFERENCE TO RELATED APPLICATIONS

This application claims the benefit of priority to U.S. Provisional Application No. 63/260,667 filed Aug. 27, 2021, which is incorporated herein by reference in its entirety for all purposes.

### BACKGROUND

### 1. Technical Field

The present disclosure relates to systems, methods, and devices for loading suture and/or shuttles into suture devices (also referred to as suture manipulating devices).

### 2. Description Of Related Art

Suture devices can pass suture and/or shuttles through tissue but may need to be loaded with suture and/or a shuttle before use.

A need still exists to load suture and/or shuttles into suture devices without damaging the suture and/or the shuttle during the loading process.

### SUMMARY

This disclosure relates generally to tissue piercers and suture devices and methods of loading and/or unloading the same.

A loader is disclosed. The loader can have a body. The body can have a device space. The loader can have a loader control. The loader can have a shuttle. The shuttle can be moveable from a shuttle first position to a shuttle second position via the loader control. A device can be positionable in the device space. When the device is positioned in the device space, the shuttle can be moveable from the shuttle first position to the shuttle second position. The shuttle can be more contracted when the shuttle is in the shuttle second position than when the shuttle is in the shuttle first position.

A loader is disclosed. The loader can have a body. The body can have a device space. The loader can have a loader control. The loader can have a deflector. The loader can have a shuttle. The shuttle can be contractible and expandable. The shuttle can be contractible via the deflector. When the shuttle is in contact with the deflector, the shuttle can have a contracted configuration. A device can be positionable in the device space. When the device is positioned in the device space and the shuttle has the contracted configuration, the shuttle can be moveable into the device via the loader control.

A method of loading and/or unloading a device is disclosed. The method can include loading a shuttle and/or a suture into the device, and/or unloading the shuttle and/or the suture from the device. Loading the shuttle can include contracting and/or expanding the shuttle. Unloading the shuttle can include contracting and/or expanding the shuttle.

### BRIEF DESCRIPTION OF THE FIGURES

The drawings shown and described are exemplary embodiments and non-limiting. Like reference numerals indicate identical or functionally equivalent features throughout.

FIGS. 1a, 1b and 1c are perspective, top and side views, respectively, of a variation of the suture passing device.

FIGS. 2a and 2b are a distant and close-up view, respectively, of a variation of the shuttle in a straight configuration.

FIG. 2c is a close-up view of the variation of the shuttle from FIGS. 2a and 2b in a curved configuration.

FIG. 3a is a close-up, perspective, partial see-through view of the distal end of a variation of the suture passing device attached to a length of a suture.

FIG. 3b is a close-up view of a portion of FIG. 3a.

FIGS. 4a and 4b are close-up perspective and side views, respectively, of the distal end of a variation of the suture passing device in a closed configuration.

FIG. 4c is a close-up of the distal end of FIGS. 4a and 4b.

FIG. 4d is a close-up perspective view of the distal end of the device of FIG. 4a in a closed configuration.

FIGS. 5A and 5B illustrate variations of the shuttle.

FIGS. 6A through 6F are bottom and side perspective, partial see-through (the upper jaw is see-through), longitudinal cross-section, partial cut-away close-up, and partial cut-away views, respectively, of the distal end of a variation of the device with the jaws in an opened configuration with the shuttle and pushers in various positions, and with the compression cover not shown in FIG. 6F for illustrative purposes.

FIG. 7A is a side perspective view of a variation of the distal end of device with the jaws in a closed configuration with the shuttle in the upper jaw and not engaged in the lower jaw.

FIGS. 7B and 7C are longitudinal cross-section and side perspective views, respectively, of the device of FIG. 7A with the shuttle in the top and bottom jaws. FIG. 26 b does not show the pushers for illustrative purposes.

FIG. 7D is a partial cut-away view of FIG. 7C.

FIG. 8A illustrates a variation of the shuttle in a lower jaw with half the lower jaw shown transparent.

FIG. 8B illustrates a variation of the shuttle in a lower jaw with half the lower jaw shown transparent.

FIG. 9A illustrates a variation of the shuttle in an upper jaw with half the upper jaw shown transparent.

FIG. 9B illustrates a variation of the shuttle in an upper jaw with half the upper jaw shown transparent.

FIG. 10 illustrates a variation of the device with half the lower and upper jaws shown transparent.

FIG. 11A illustrates a perspective view of a variation of a shuttle.

FIG. 11B illustrates a bottom view of the shuttle of FIG. 11A.

FIG. 11C illustrates a side view of the shuttle of FIG. 11A.

FIG. 11D illustrates a front perspective view of a variation of a shuttle.

FIG. 11E illustrates a rear perspective view of the shuttle of FIG. 11D

FIG. 11F illustrates a front perspective view of a variation of a shuttle.

FIG. 11G illustrates a rear perspective view of the shuttle of FIG. 11F.

FIG. 12A illustrates a side view of a variation of the device with half the device shown transparent.

FIG. 12B illustrates a perspective view of the device of FIG. 12A.

FIG. 13A illustrates a variation of the device.

FIG. 13B illustrates a variation of the device.

FIG. 13C illustrates a variation of a handle of the device.

FIG. 13D illustrates a variation of a handle of the device.

FIG. 13E illustrates a variation of a handle of the device.

FIG. 14A illustrates a top view of a variation of a loader.

FIG. 14B illustrates a top view of a variation of the loader of FIG. 14A.

FIG. 14C illustrates a top view of a variation of the loader of FIG. 14A.

FIG. 14D illustrates a perspective view of a variation of the loader of FIG. 14A.

FIG. 14E illustrates a top view of a variation of the loader of FIG. 14A.

FIG. 14F illustrates a perspective view of a variation of the loader of FIG. 14A.

FIG. 15A illustrates a top view of a variation of the loader of FIG. 14A.

FIG. 15B illustrates a perspective view of a variation of the loader of FIG. 14A.

FIG. 15C illustrates a magnified perspective view of the loader of FIG. 15B.

FIG. 15D illustrates a magnified perspective view of the loader of FIG. 15C.

FIG. 15E illustrates a magnified perspective cross-sectional view of the loader of FIG. 15D.

FIG. 15F illustrates a magnified perspective cross-sectional view of the loader of FIG. 15E.

FIG. 16 illustrates a top view of a variation of the loader of FIG. 14A.

FIG. 17A illustrates a top view of a variation of the loader of FIG. 14A.

FIG. 17B is a magnified view of the loader of FIG. 17A at section 17B-17B.

FIG. 17C is a perspective view of FIG. 17B.

FIG. 18 is a bottom view of a variation of the loader of FIG. 14A.

FIG. 19A is a perspective view of a variation of a loader control.

FIG. 19B is a perspective view of the loader control of FIG. 19A with suture.

FIG. 20A is a perspective view of a variation of a loader control in an open configuration.

FIG. 20B is a perspective view of the loader control of FIG. 20A in a first closed configuration.

FIG. 20C is a perspective view of the loader control of FIG. 20A in a second closed configuration.

FIG. 20D is a top view a component of the loader control of FIG. 20A.

FIG. 20E is a side view of the component of FIG. 20D.

FIG. 20F is a magnified view of section 20F-20F in FIG. 20E.

FIG. 20G is a front view of the component of FIG. 20E taken along line 20G-20G.

FIG. 20H is a bottom view of the component of FIG. 20D.

FIG. 20I is a bottom view of a component of the loader control of FIG. 20A.

FIG. 20J is a side view of the component of FIG. 20I.

FIG. 20K is a cross-sectional view of the component of FIG. 20J taken along line 20K-20K.

FIG. 20L is a front view of the component of FIG. 20J taken along line 20L-20L.

FIG. 20M is a top view of the component of FIG. 20I.

FIG. 20N is a cross-sectional view of the component of FIG. 20M taken along line 20N-20N.

FIG. 20O is a perspective view of the component of FIG. 20I.

FIG. 21A illustrates a top view of a variation of a loader.

FIG. 21B illustrates a top view of a variation of the loader of FIG. 21A.

FIG. 21C illustrates a top view of a variation of the loader of FIG. 21A.

FIG. 21D illustrates a top view of a variation of the loader of FIG. 21A.

FIG. 21E illustrates a top view of a variation of the loader of FIG. 21A.

FIG. 21F illustrates a top view of a variation of the loader of FIG. 21A.

FIG. 21G illustrates a top view of a variation of the loader of FIG. 21A.

FIG. 21H is a magnified view of section 21H-21H in FIG. 21G.

FIG. 21I illustrates a magnified perspective view of the loader of FIG. 21H.

FIG. 21J illustrates a magnified perspective view of the loader of FIG. 21H.

FIG. 21K illustrates a magnified perspective view of the loader of FIG. 21H.

FIG. 21L illustrates a magnified perspective view of the loader of FIG. 21H.

FIG. 21M illustrates a magnified perspective view of the loader of FIG. 21H.

FIG. 21N illustrates a side view of a variation of the loader of FIG. 21A.

FIG. 21O illustrates a side view of a variation of the loader of FIG. 21A.

FIG. 21P illustrates a side view of a variation of the loader of FIG. 21A.

FIG. 21Q illustrates a bottom perspective view of a variation of the loader of FIG. 21A.

FIG. 21R illustrates a bottom perspective view of a variation of the loader of FIG. 21A.

FIG. 21S illustrates a top perspective view of a variation of the loader of FIG. 21A.

### DETAILED DESCRIPTION

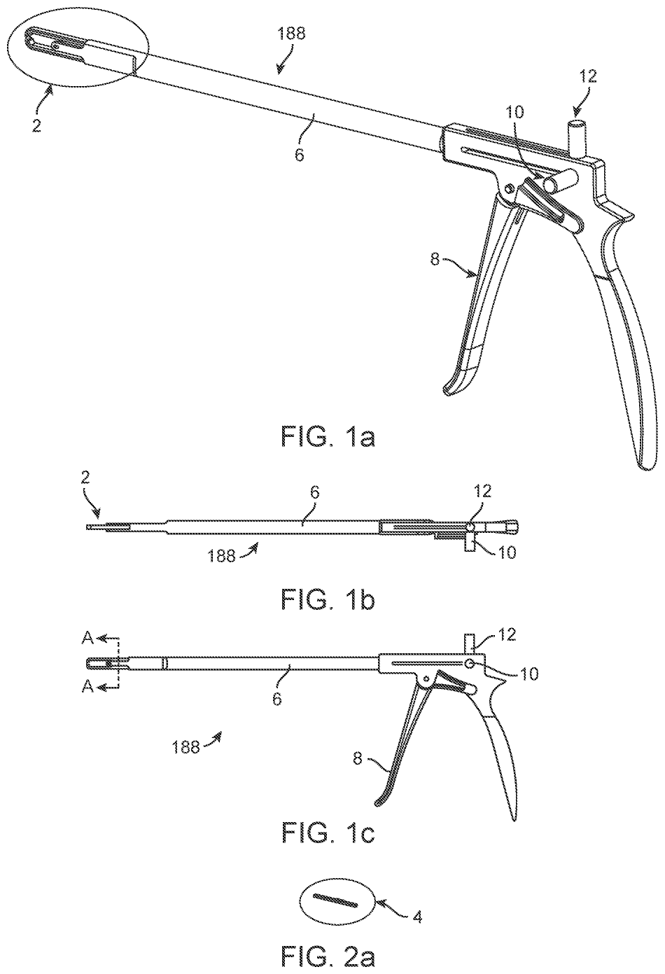

FIGS. 1a through 1c illustrate a suture passing device 188 that can be used to pass suture 70 through soft or hard tissue 74 with or without removing the device 188 or the suture 70 from the target site while creating one or more complete stitches.

The suture passing device 188 can have an ergonomic handle 104, a sliding tube actuator 6, and a distal end 2. The ergonomic handle 104 can be used to control the distal end 2. The ergonomic handle 104 can have a side knob 10. The ergonomic handle 104 can have a top knob 12. The top knob 12 and/or the side knob 10 can individually or in concert, advance and/or retract the upper 86 and/or lower pusher 76.

The sliding tube actuator 6 can have an outer compression cover 34 and an inner rod (not shown due to obstruction by the outer compression cover 34). The inner rod can be fixedly attached to the handle 104 and the proximal end of the jaw structure 28. The outer compression cover 34 can be radially outside of the inner rod. The outer compression cover 34 can be actuated by the handle 104, for example be distally and proximally translated with respect to the handle 104 when the trigger 8 is squeezed or released.

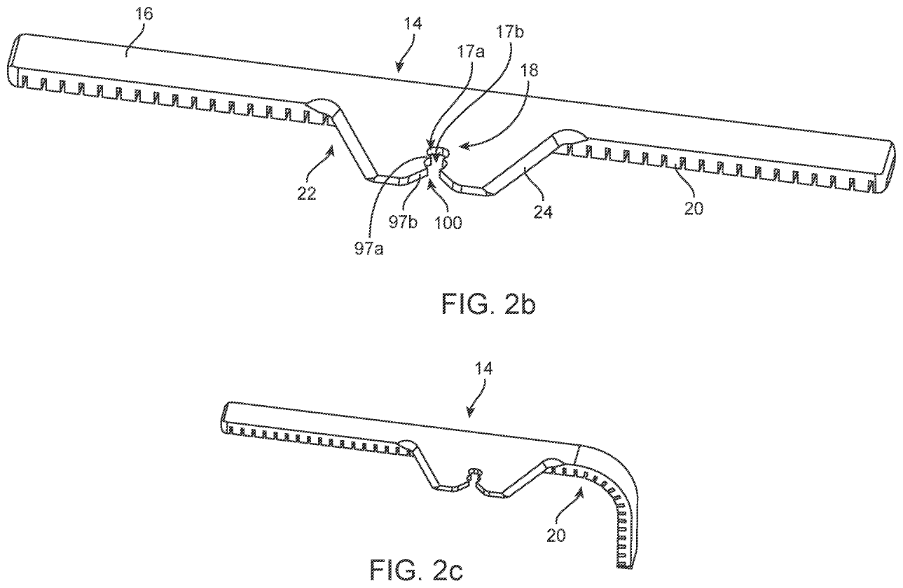

FIGS. 2a and 2b illustrate that the device 188 can have a sliding ribbon shuttle 14 or needle held within the device 188. The shuttle 14 can have an elongated shuttle rail 16. The shuttle rail 16 can have numerous slits 20 along one or both sides of the shuttle rail 16. The slits 20 can be positioned at regular or irregular length intervals along the rail 16.

The shuttle 14 can have a suture holder 18 extending laterally from the rail 16. The shuttle 14, for example the suture holder 18, can extend out of the lateral side slot 72 of the arm structure. The suture holder 18 can extend from the left and/or right side of the device 188. The distal end 2 of the device 188 can be reversible so the suture holder 18 can be switched from one side of the device 188 to the other side of the device 188. The suture holder 18 can have a generally flat, isosceles trapezoid configuration. The suture holder 18 can have a suture holding notch 100. The notch 100 can have an inner hole 17a, an outer hole 17b contiguous with the inner hole 17a, and a first cleat 97a positioned between the inner hole 17a and the outer hole 17b. The notch 100 can have a second cleat 97b on the side of the outer hole away from the inner hole. The notch 100 can be configured to secure to suture 70. For example, the suture 70 can be compressed and friction fit in the inner cleat 97a.

The suture holder 18 can have a front leading edge and a rear leading edge. The edges can be slanted at a right or non-right angle with respect to the longitudinal axis of the rail 16. One or both of the edges can be sharpened to be traumatic to tissue 74, for example to cut through soft tissue 74. The edges can cut through tissue 74, allowing the suture holder 18 to pull the suture 70 through the tissue 74 immediately behind the respective edge.

The shuttle 14 can be made from a flexible polymer, such as PEEK, a resilient metal such as Nitinol, any material disclosed herein or combinations thereof. The shuttle 14 can be made from a molded polymer. The shuttle 14 can be pre-curved, for example to reduce resistance when going around curves in the tracks.

FIG. 2c illustrates that the rail 16 can curve at the locations of the slits 20, and/or the rail 16 can be pre-curved.

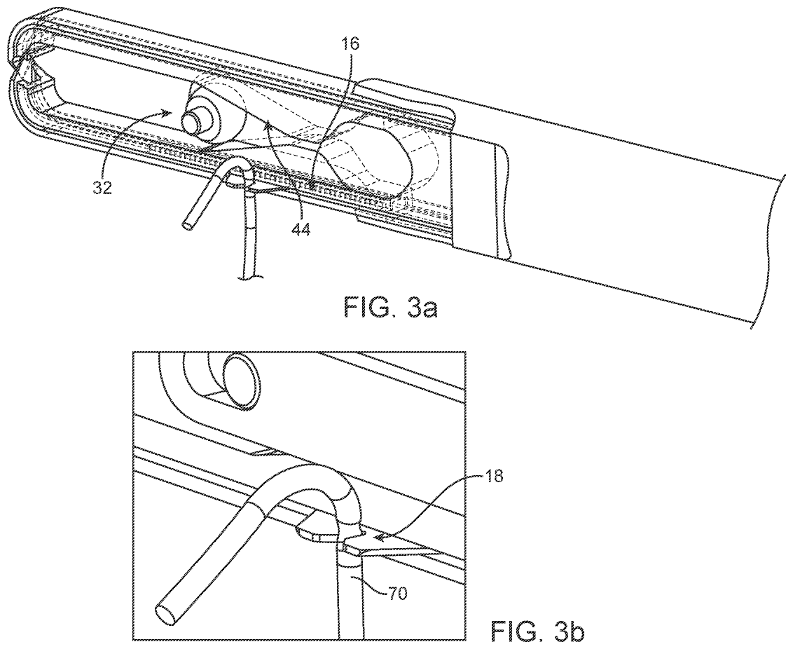

FIGS. 3a and 3b illustrate that the suture passing device 188 can capture or releasably attach to the suture 70 in the inner and/or outer cleats 97a and/or 97b of the suture holder 18. The suture 70 can be loaded or held laterally of the jaw structure 28, out of plane with the rotation of the jaws. The device 188 can make multiple passes of the suture 70 through the tissue 74 without extracting or reloading the suture passing device 188. The jaw structure 28 can resiliently deform open at the proximal end of the jaw structure 28, having no hinge. The jaws can be opened and/or closed with no mechanical pivots or linkages in the jaw structure 28.

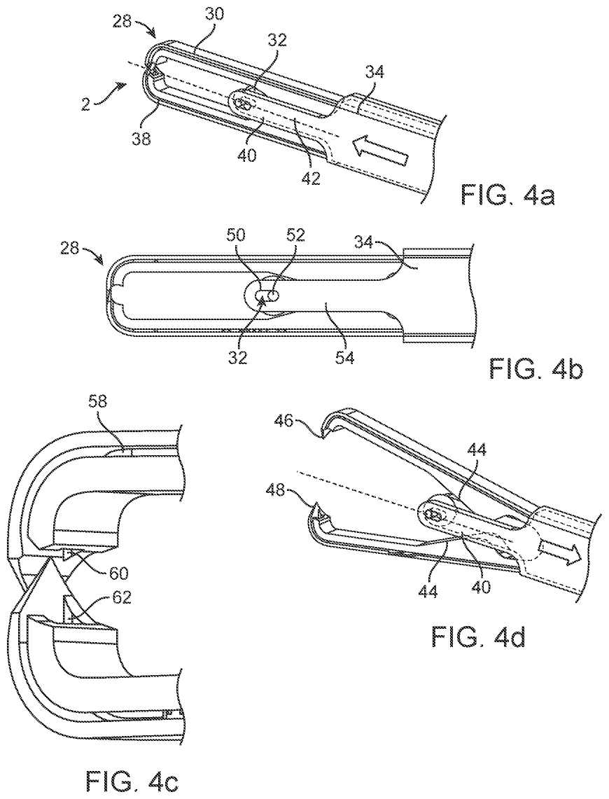

FIG. 4a illustrates that the suture passer device 188 can have a jaw structure 28 with a top jaw 30 and a bottom jaw 38. The entire jaw structure 28 can be an integral piece of material, such as a single molded, cast, or cut element of Nitinol, other resilient metal or polymer, any other material listed herein, or combinations thereof. The jaw structure 28 can be configured to be in an opened configuration (as shown in FIG. 4d) when in an unbiased configuration (i.e., when no external forces are applied).

The jaw structure 28 can have a jaw structure longitudinal axis 42. Each jaw can also have a respective jaw longitudinal axis along the jaw.

The inside channel of the compression cover 34 can be sized and shaped to fit over the jaw structure 28 with minimum clearance when the jaw structure 28 is in a closed configuration. When the compression cover is translated distally 138 with respect to the jaw structure 28, as shown by arrow, the compression cover 34 can press the top and bottom jaws 38 toward the jaw structure longitudinal axis 42. The jaw structure 28 can be fully compressed into a closed configuration, as shown in FIGS. 4a through 4c. In this way, when an actuation lever such as the trigger 8 is actuated, the channel or compression cover 34 can advance to cam closed the jaws. The jaws can pre-pierce the tissue and establish a continuous track for the shuttle to pass through the tissue.

The compression cover 34 can be attached to an opening ball 32 positioned between the first and second jaws.

FIG. 4b illustrates that the opening ball 32 can be rotatably or fixedly attached to a ball axle 52 passing laterally through the opening ball 32. The ball axle 52 can extend out from the lateral sides of the ball 32. The ball axle 52 can be slidably received by axle slots 50 formed through distal arms 54 or extensions 138 of the compression cover 34. When the jaw structure 28 is in a closed configuration, the ball axle 52 can abut and interference fit against the proximal end of the axle slot 50, for example to prevent overextension of the compression cover 34 over the jaw structure 28. When the jaw structure 28 is in an opened configuration, the ball axle 52 can abut and interference fit against the distal end 2 of the axle slot 50, for example to prevent overrotation of the jaws and/or pulling the ball 32 past the ramps 44 on the inside of the jaw structure 28.

FIG. 4c illustrates that the bottom track 66 can distally terminate in a bottom track port 62. The top track 64 can distally terminate at a top track port 60. The top track port 60 can align with and be adjacent to (as shown) or in contact with the bottom track port 62 when the jaw structure 28 is in a closed configuration with the first jaw tip 46 interdigitating with the second jaw tip 48. The tracks of the upper jaw 78 and bottom jaw 38 can form a continuous path when the jaw structure 28 is in a closed configuration. The first jaw tip 46 can interdigitate with and be adjacent or in contact with the second jaw tip 48 when the jaw structure 28 is in a closed configuration.

FIG. 4d illustrates that that compression cover 34 can be translated proximally 126, as shown by arrow, with respect to the jaw structure 28. The ball axle 52 can slide to the distal end 2 of the axle slot 50. The axle slot 50 can then pull the ball axle 52, and therefore the opening ball 32, proximally. The opening ball 32 can then press against the inside surface ramp 44 of the first jaw and/or second jaw. The first jaw tip 46 and/or second jaw tip 48 can then rotate away from the opposing jaw tip. The jaw structure 28 can then be in an opened configuration, as shown.

The proximal ends of the jaws can be rigid or flexible, for example to bend around the opening of the compression cover 34 when the jaws are in an opened configuration. The entire jaws or just the proximal ends of the jaws can be made from Nitinol, for example with the distal ends of the jaws made from stainless steel.

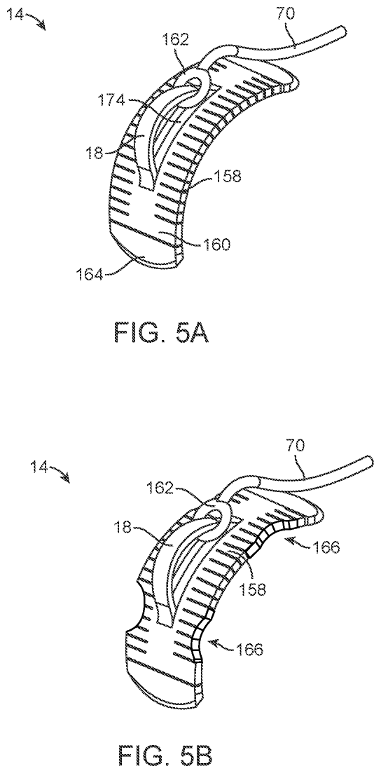

FIG. 5A illustrates that the suture holder 18 can be an arc integral with the shuttle spine 160. For example, the shuttle 14 can be made from a single panel of material (e.g., metal). The lateral sides of the suture holder 18 can be cut, and the longitudinal ends can remain integrated with the shuttle spine 160. The suture holder 18 can then be bent or otherwise deformed away from the plane of the shuttle spine 160, for example forming an arc away from the plane of the shuttle spine 160.

The suture 70 can have a suture loop 162 at the terminal end of the suture 70. The suture loop 162 can extend around and completely or partially circumscribe the suture holder 18. The remainder of the suture 70 can be integral with the suture loop 162, or can removably attached to the suture loop 162. The suture loop 162 can be circular or oval.

FIG. 5B illustrates that the shuttle 14 can have one or more shuttle notches 166 or cut-outs. For example, the shuttle 14 can have two shuttle notches 166 on each lateral site of the shuttle. The shuttle notches 166 can be even longitudinally spaced and distributed along the shuttle 14. The shuttle notches 166 can be curved. The sides of the shuttle 14, other than at the notches, can be straight.

A radius of curvature of the shuttle notch 166 can be from about 1 mm to about 2 mm.

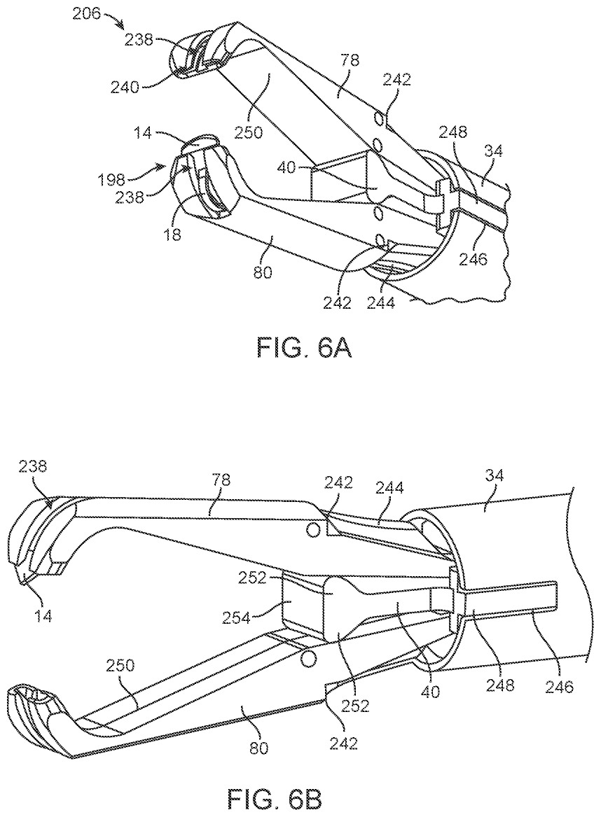

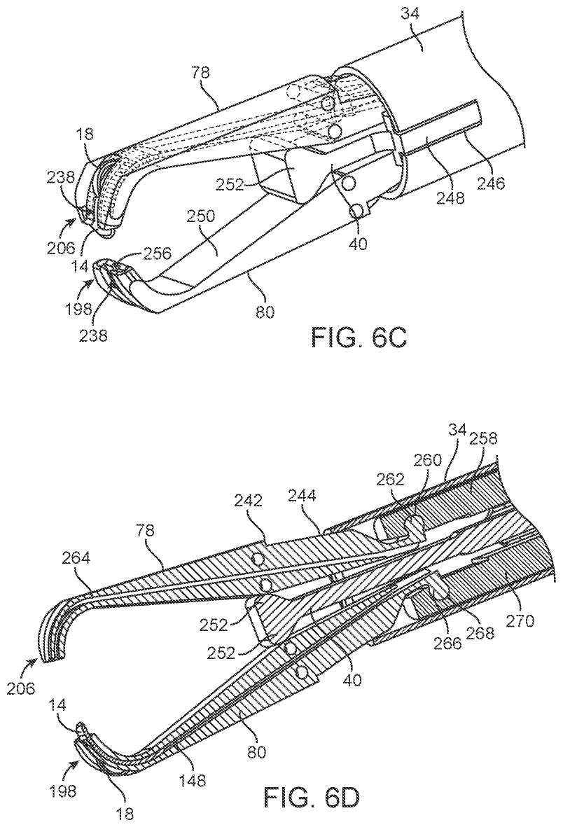

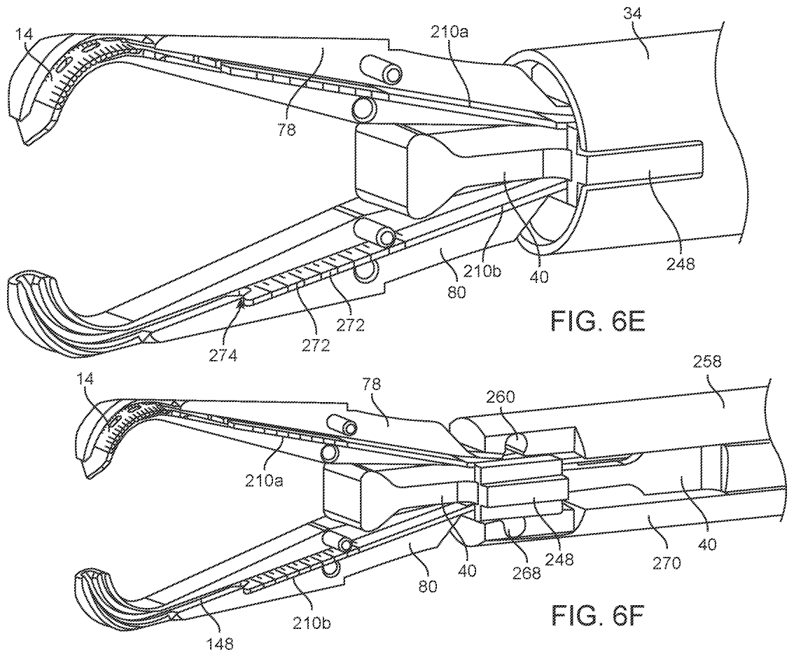

FIGS. 6A through 6F illustrate that the upper jaw tip 206 and/or lower jaw tip 198 can have suture holder slots 238. The suture holder slots 238 can extend medially along the outer surface of the respective jaw tip. The suture holder slot 238 can extend from the outer surface of the jaw tip to the respective track. The suture holder 18 can be accessible through or extend out of the suture holder slot 238. The suture 70 (not shown) can attach to or be integral with the suture holder 18 in or outside of the suture holder slot 238.

The upper track 264 can distally terminate at an upper jaw tip shuttle port 240. The lower track 148 can distally terminate at a lower jaw tip shuttle port 256. The shuttle 14 can extend out of or into, and pass through each of the shuttle 14 ports. During use, the sharpened shuttle tip 164 extending out of the shuttle port can pierce, cut and dissect tissue 74 when the jaws are rotated to a closed configuration.

The upper jaw 78 and/or lower jaw 80 can have a jaw stop 242. The jaw stop 242 can be a feature, shape or configuration that can abut and stop the distal translation of the compression cover 34 with respect to the jaws. For example, the distal terminal end of the compression cover 34 can abut the jaw stops 242 when the jaws are in a closed configuration.

The radially inner surface of the jaws can have radially inner slopes 250.

The upper jaw 78 and/or lower jaw 80 can have a jaw slide 244. The jaw slide 244 can be a radially outer surface of the jaws between the jaw stops 242 and the compression cover 34 when the compression cover 34 is in a proximally retracted 126 position with respect to the jaws and/or the jaws are in an opened configuration. The jaw slide 244 can increase in radius from the jaw structure longitudinal axis 42 in the distal longitudinal direction (e.g., the larger the longitudinal dimension of the jaw slide 244, the larger the radial dimension of the jaw slide 244). When the compression cover is translated distally 138 with respect to the jaws, the radially inner distal edge of the compression cover 34 can slide along the jaw slide 244, and press the jaw slide 244 toward the jaw structure longitudinal axis 42. A radially compressive force delivered from the compression cover 34 to the jaw slide 244 can create a torque in the respective jaw, rotating the respective jaw toward the jaw structure longitudinal axis 42 and the opposing jaw.

The device 188 can have a jaw control extension 40. The jaw control extension 40 can extend along the jaw structure longitudinal axis 42. The jaw control extension 40 can extend between the jaws proximal to the jaw tips. The jaw control extensions 40 can terminate in a jaw control extension head 254.

The jaw control extension head 254 can have one or two lobes or cams. Each lobe can extend from the longitudinal axis of the jaw control extension 40 toward a jaw. The lobes can act similarly to the opening roller ball shown in FIGS. 4a, 4d, and elsewhere herein. The upper jaw 78 and lower jaw 80 can have upper and inner jaw radially inner slopes 250, respectively. The inner slopes can be the radially inner surfaces of the jaws proximal to the jaw tips and distal to the jaw control extension head 254 when the jaw control extension head 254 is in a proximally retracted position with respect to the jaws. The radially inner slope 250 can increase in radius from the jaw structure longitudinal axis 42 in the distal longitudinal direction (e.g., the larger the longitudinal dimension of the radially inner slope 250, the larger the radial dimension of the radially inner slope 250). When the jaw control extension 40 is proximally translated or retracted with respect to the jaws, the lobes can slide against the radially inner slopes 250 of the jaws and press the jaws away from each other into an open configuration.

When the jaws are in an open configuration, the compression cover 34 can be positioned at or proximally past the proximal end of the jaw slides 244, and the jaw extension head can be positioned at or proximally past the proximal end of the radially inner slopes 250.

The jaw control extension 40 can be attached to or integral with a control rail 248. The control rail 248 can extend radially from one or both lateral sides of the jaw control extension 40, for example in a plane at a right angle to a plane defined by the opposing jaws or the opposing extension head lobes 252.

The compression cover 34 can have a control rail slot 246. The control rail slot 246 can extend to the distal terminal end of the compression cover 34. The control rail 248 can be fixed to or longitudinally translate within the control rail slot 246. The control rail 248 can interference fit, abut or stop against the proximal end of the control rail slot 246, for example when the control rail 248 is in a proximal or distal longitudinal position with respect to the jaws. The control rail 248 can move longitudinally in unison (i.e., coincidentally) with the compression cover 34 in the distal and/or longitudinal directions. The control rail 248 can move longitudinally in unison with the jaw control extension 40 in the distal and/or longitudinal directions.

The device 188 can have an upper socket arm 258 and a lower socket arm 270 radially inside of the compression cover 34. The upper socket arm 258 and lower socket arm 270 can be a single integrated element (e.g., a hollow cylinder) or separate elements. The upper socket arm 258 can be opposite the lower socket arm 270. The upper socket arm 258 can be translatably fixed (i.e., mechanically attached to translate in unison) to the lower socket arm 270. The jaw control extension 40 can extend longitudinally between the upper 258 and lower socket arms 270 or within a hollow channel inside a unitary socket arm (comprising the upper 258 and lower socket arms 270 as an integrated element). The distal terminal ends of the socket arms can extend to or proximal to the distal terminal end of the compression cover 34 when the jaws are in an open configuration.

The proximal terminal end of the upper jaw 78 can have a laterally elongated upper jaw bearing 262. The upper jaw bearing 262 can extend radially outward from the remainder for the proximal end of the upper jaw 78.

The distal end 2 of the upper socket arm 258 can have a laterally elongated upper jaw socket 260. The upper jaw socket 260 can open medially and have a diameter approximately equal to or slightly larger than the diameter of the upper jaw bearing 262.

An upper jaw 78 hinge can have the upper jaw bearing 262 and the upper jaw socket 260. The upper jaw 78 can rotate around the transverse axis of the upper jaw bearing 262. The upper jaw bearing 262 can rotate in the upper jaw socket 260.

The proximal terminal end of the lower jaw 80 can have a laterally elongated lower jaw bearing 266. The lower jaw bearing 266 can extend radially outward from the remainder for the proximal end of the lower jaw 80.

The distal end 2 of the lower socket arm 270 can have a laterally elongated lower jaw socket 268. The lower jaw socket 268 can open medially and have a diameter approximately equal to or slightly larger than the diameter of the lower jaw bearing 266.

A lower jaw 80 hinge can have the lower jaw bearing 266 and the lower jaw socket 268. The lower jaw 80 can rotate around the transverse axis of the lower jaw bearing 266. The lower jaw bearing 266 can rotate in the lower jaw socket 268.

The upper 86 and/or lower pushers 76 can have entire lengths or only distal ends 2 that can have articulated segmentations 286. The articulated segments 286 can rotate with respect to each other around an axis perpendicular to the longitudinal axis of the respective pusher. The articulated segmentations 286 can be connected by a discrete hinge (e.g., a pin or snap connection) or can be longitudinally coincidental or longitudinally alternating lateral slots cut into the sides of the pusher, similar to the shape of the shuttle lateral slots 158. The proximal end of either or both upper 86 and lower pushers 76 can have a continuous, non-segmented, flat, uniform ribbon of material.

Each of the upper 86 and/or lower pushers 76 can have distal terminal ends that can have a shuttle seat 274. The shuttle seat 274 can be an inverse shape to the shape of the shuttle tip 164. For example, if the shuttle tip 164 has an angled end, the shuttle seat 274 can have the opposite angle. If the shuttle tip 164 has a convex curved end, the shuttle seat 274 can have a concave curved end with the same radius of curvature as the shuttle tip 164.

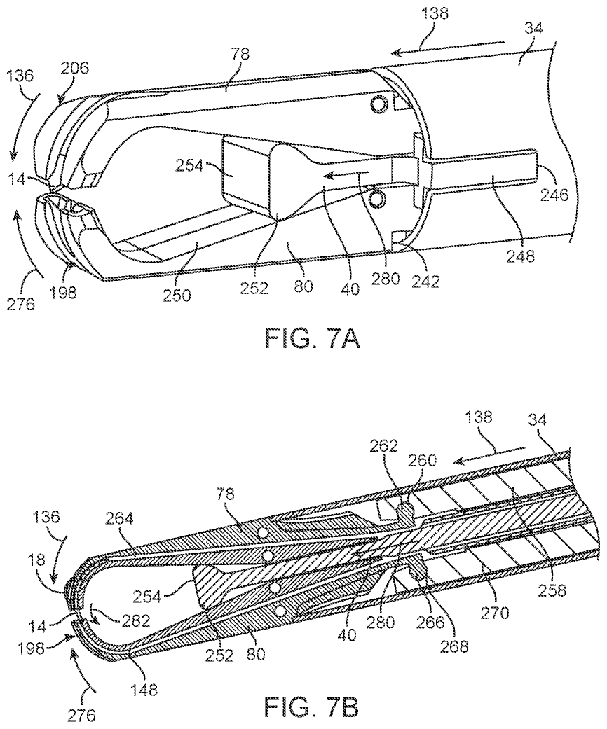

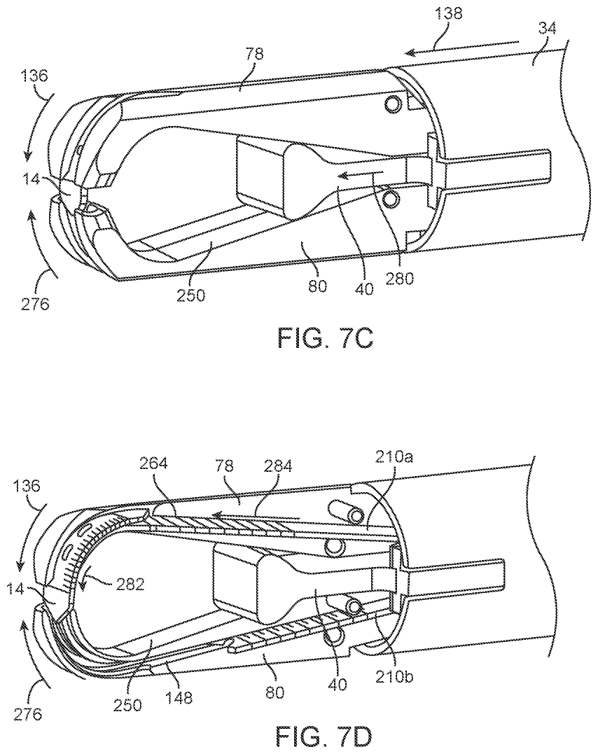

FIGS. 7A through 7D illustrate that the compression cover 34 can be distally translated, as shown by arrow, with respect to the jaws. The compression cover 34 can deliver translational force through the edges of the control rail slot 246 to the control rail 248. The control rail 248 can deliver the translational force to the jaw control extension 40. The jaw control extension 40 can translate distally, as shown by arrow, concurrently with the compression cover 34. The compression cover 34 can translate 138 over the jaw slides 244, pressing radially inward on the jaw slides 244. The jaw control extension head 254 can move distally with respect to the jaws, as shown by arrow 280, for example, allowing the closure of the jaws without interference fitting or abutting against the jaw control extension head 254. The upper jaw 78 and/or lower jaw 80 can rotate radially inward, as shown by arrows.

When the jaws are in a closed configuration, the compression cover 34 can be positioned at or adjacent to the jaw stop 242, and the jaw extension head can be positioned at or proximally past the proximal end of the radially inner slopes 250.

When the jaws are in a closed configuration, if the shuttle 14 is in the upper track 264, the upper pusher 86 can translate distally through the upper track 264. The distal terminal end of the upper pusher 86 can abut the shuttle 14. The upper pusher 86 can then push the shuttle 14 through the upper track 264, out the upper jaw tip shuttle port 240 and into the lower jaw tip shuttle port 256.

When the jaws are in a closed configuration, if the shuttle 14 is in the lower track 148, the lower pusher 76 can translate distally through the lower track 148. The distal terminal end of the lower pusher 76 can abut the shuttle 14. The lower pusher 76 can then push the shuttle 14 through the lower track 148, out the lower jaw tip shuttle port 256 and into the upper jaw tip shuttle port 240.

When the shuttle 14 is pushed from the upper track 264 to the lower track 148 or vice versa, the shuttle 14 can be curvilinearly translated 282, as shown by arrow, following the paths of the upper track 264 and the lower track 148.

When the jaws are in a closed configuration, the shuttle 14 can move from the upper jaw 78 to the lower jaw 80, as shown by arrow, back to the upper jaw 78, and can repeat the motion from the upper jaw 78 to the lower jaw 80, and optionally from the lower jaw 80 to the upper jaw 78 one, two or more times.

The device 188 can have a pusher lockout that can prevent translation of the pushers and the shuttle 14 when the jaws are in an open configuration.

The device 188 can have a jaw lockout preventing opening of the jaws when either of the pushers is extended out of the respective jaw tip shuttle port and/or when the shuttle 14 is concurrently in the upper jaw 78 and the lower jaw 80.

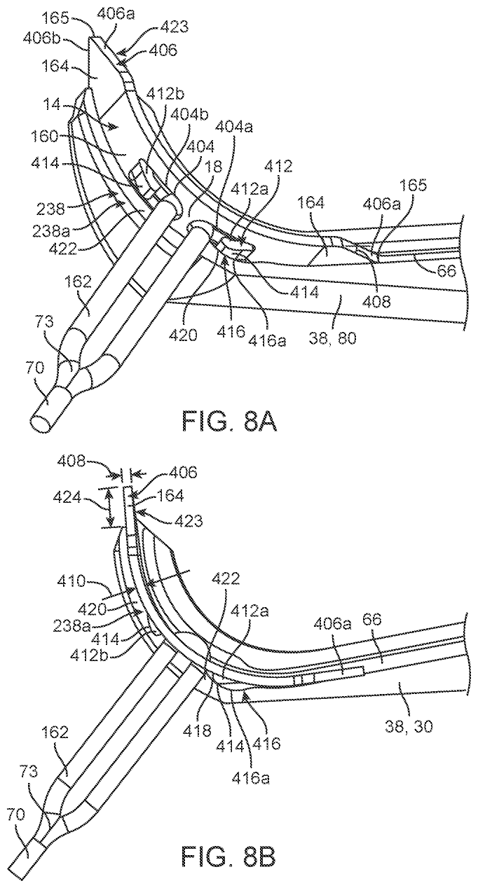

FIG. 8A illustrates that the suture holder 18 can be attached to or integrated with the shuttle 14. For example, the suture holder 18 can be a bridge integrated with the shuttle spine 160. A portion of the shuttle spine 160 can define the suture holder 18. As another example, the suture holder 18 can be removably attached to the shuttle 14. The suture holder 18 can extend between a shuttle first lateral side and a shuttle second lateral side. The suture holder can extend between a shuttle first longitudinal side and a shuttle second longitudinal side. The suture holder 18 can be in the longitudinal center of the shuttle 14, on a proximal end of the shuttle 14, or on a distal end of the shuttle 14. A center of the suture holder 18 can be in the transverse center of the shuttle 14, on a first lateral side of the shuttle, or on a second lateral side of the shuttle. The suture holder 18 can be in the plane of the shuttle spine 160, extend away from the plane of the shuttle spine 160, or both. For example, FIG. 8A illustrates that the suture holder 18 does not extend away from the plane of the shuttle spine 160. The plane of the suture holder 18 can be flush with or coincident with the plane of the shuttle spine 160. This can advantageously allow the shuttle and suture holder 14, 18 to take up less space, thereby minimizing the trauma to surrounding tissue as the shuttle 14 is passed between the upper and lower jaws 30, 38 since it brings the base of the suture 70 closer to the shuttle 14. With the suture 70 closer to the shuttle 14, the force of the suture 70 against surrounding tissue is reduced as compared to when the suture 70 is connected to a structure out of the plane of the shuttle spine 160 (e.g., the suture holder 18 of FIGS. 5A and 5B). The shuttle and suture holder 18 can be a monolithic structure. The suture loop 162 can extend around and completely or partially circumscribe the suture holder 18. The remainder of the suture 70 can be integral with the suture loop 162, or can removably attached to the suture loop 162. The suture loop 162 can be attached to or integrated with the suture 70 at a suture junction 73. The suture junction 73 can be a knot, a braid, or both. The suture loop 162 can be circular, oval, or stadium-shaped.

The shuttle 14 can have zero, one, or multiple suture holes 404, for example, 0 to 4 or more suture holes 404, including every 1 suture hole increment within this range. For example, FIG. 8A illustrates that the shuttle 14 can have a first suture hole 404a and a second suture hole 404b. The shuttle holes 404 (e.g., first and second suture holes 404a, 404b) can have a regular or irregular shape, for example, curved, polygonal, or both. The suture holes 404 can be defined by one or more curved surfaces or curved edges, for example, one or more curved surfaces or curved edges of the shuttle 14. The suture holes 404 can be defined by one or more flat surfaces or straight edges, for example, one or more flat surfaces or straight edges of the shuttle 14. The suture holes 404 can have a cross-sectional shape of a circle, ellipse, rectangle, stadium, horseshoe, star, slot, or any combination thereof. The suture holes 404 can have such cross-sectional shapes when the shuttle is curved or flat. The suture holes 404 can have a constant cross-sectional area or a tapered cross-sectional area.

The shuttle tips 164 can be beveled, non-beveled, or both. For example, FIG. 8A illustrates that the shuttle tips 164 can be non-beveled. The shuttle tips 164 can have one or multiple tip surfaces 406, for example, 1 to 4 or more tip surfaces 406, including every 1 tip surface increment within this range (e.g., 1 tip surface, 2 tip surfaces). For example, FIG. 8A illustrates that the shuttle tips 164 can have a first tip surface 406a (e.g., a first non-beveled tip surface as shown in FIG. 8A) and a second tip surface 406b (e.g., a second non-beveled tip surface as shown in FIG. 8A). The non-beveled portion of the shuttle tips 164 can advantageously improve the force transfer from the upper and lower pushers 86, 76, thereby making it easier for the pushers 86 and 76 to push against the shuttle 14. A larger component of the force from the pushers (e.g., pushers 86 and 76) can be transferred along the longitudinal axis of the shuttle 14 when the shuttle tip 164 has a non-beveled surface 406 as compared to a beveled surface (e.g., the beveled surfaces shown in FIGS. 5A and 5B at the tips of the shuttle). For beveled surfaces, a portion of the force applied to the shuttle 14 is directed against the surfaces that define the lower and upper tracks 66, 64 perpendicularly away from the beveled surface. When a pusher applies a longitudinal force against a beveled surface, a portion of the longitudinal force applied to the shuttle 14 by the pusher is transformed into a transverse component away from the longitudinal axis of the shuttle 14.

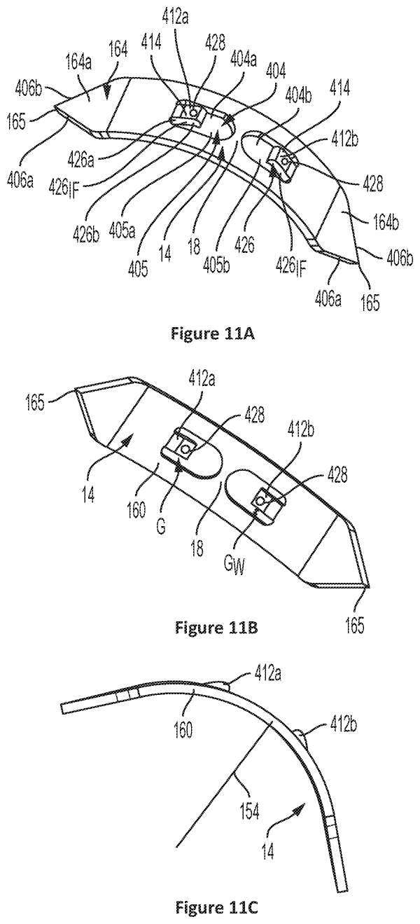

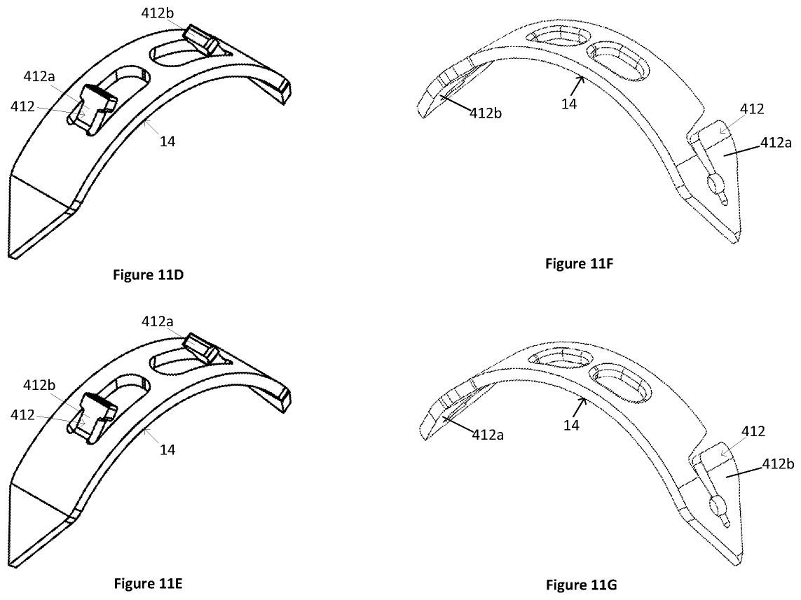

FIG. 8A illustrates that the shuttle tips 164 can be tapered to form a terminal tip 165. The terminal tip 165 can be an edge or part of a rounded or flat surface. The tapered portion of the shuttle tip 164 can be a first bevel, for example, a first transverse bevel toward a longitudinal axis (e.g., center longitudinal axis) of the shuttle 14. The non-beveled surfaces 406 can thereby form first tissue cutting surfaces that are beveled in a first direction. The bevel referred to in the preceding paragraph can refer to a second bevel, for example, a second transverse bevel angled relative to a transverse axis perpendicular to the transverse axis of the first bevel and toward a longitudinal axis (e.g., center longitudinal axis) of the shuttle 14. Such a second bevel is shown in FIGS. 11F and 11G at the tips of the shuttle, for example, without a first bevel (e.g., the first bevel shown in FIGS. 5A and 5B). The second bevels can define second beveled surfaces along the taper that face a second direction different from the first direction. The second bevels can form second tissue cutting surfaces. The shuttle tips can pierce or cut tissue. The tapered portion of the shuttle tips 164 can pierce or cut tissue. The tip surfaces 406 (e.g., tip surfaces 406a and 406b) can pierce or cut tissue. When the shuttle tips 164 have first and second bevels, the first and second bevels can pierce tissue or cut tissue. FIG. 8A illustrates that the shuttle tips 164 can be non-sharpened, meaning that while the edges are tapered to form a first cutting surface, the cutting surfaces 406 themselves can be chamfered or non-chamfered. The shuttle tips 164 can be non-sharpened and still cut or pierce tissue, where sharpened or non-sharpened can refer to the presence or non-presence of a second bevel (e.g., as shown in FIGS. 11F and 11G), respectively.

FIG. 8A illustrates that the device 188 can have one or multiple male stops 412 (also referred to as male catches, male detents, stops, catchers, detents) and one or multiple female stops 416 (also referred to as female catches, female detents, stops, catchers, detents). The device 188 can have, for example, 1-10 or more male stops 412, including every 1 male stop increment within this range (e.g., 1, 2, 3, 4 or more male stops). The device 188 can have, for example, 1-10 or more female stops 416, including every 1 female stop increment within this range (e.g., 1, 2, 3, 4 or more female stops).

The male stops 412 can be attached to or integrated with the device 188. For example, the male stops 412 can be part of, attached to, or integrated with the shuttle 14. As another example, the male stops 412 can be part of, attached to, or integrated with the jaws (e.g., jaws 30, 38, 78, 80). As yet another example, the device 188 can have some male stops 412 that are part of, attached to, or integrated with the shuttle 14 and can have some male stops 412 that are part of, attached to, or integrated with the jaws (e.g., jaws 330, 38, 78, 80). As yet still another example, the male stops 412 can be part of, attached to, or integrated with the pushers (e.g., the lower and upper pushers 76, 86).

The female stops 416 can be attached to or integrated with the device 188. For example, the female stops 416 can be part of, attached to, or integrated with the shuttle 14. As another example, the female stops 416 can be part of, attached to, or integrated with the jaws (e.g., jaws 330, 38, 78, 80). As yet another example, the device 188 can have some female stops 416 that are part of, attached to, or integrated with the shuttle 14 and can have some female stops 416 that are part of, attached to, or integrated with the jaws (e.g., jaws 330, 38, 78, 80). As yet still another example, the female stops 416 can be part of, attached to, or integrated with the pushers (e.g., the lower and upper pushers 76, 86).

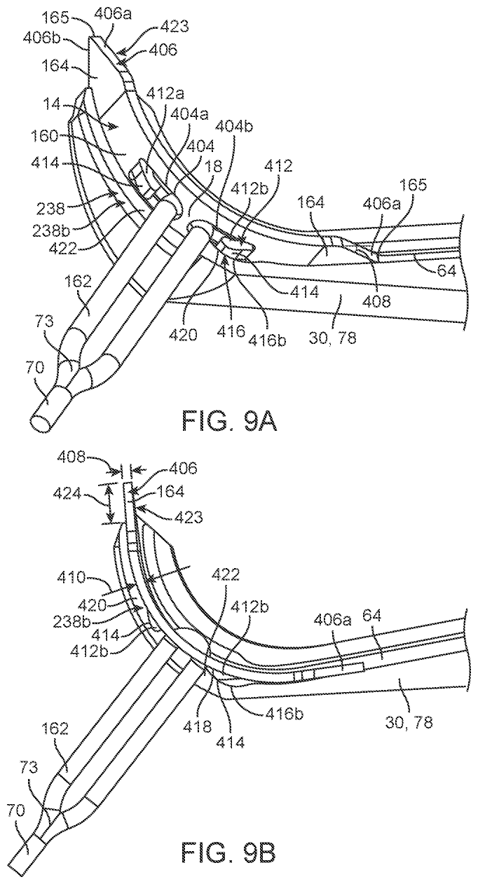

FIGS. 8A-9B illustrate, for example, that the shuttle 14 can have the male stops 412 and that the jaws (e.g., jaws 330, 38, 78, 80) can have the female stops 416. For example, FIGS. 8A-9B illustrate that the shuttle 14 can have a first male stop 412a and a second male stop 412b, that the lower jaw (e.g., jaw 38, 80) can have a first female stop 416a (also referred to as the lower jaw first female stop 416a and other similar terms) configured to releasably engage with or releasably attach to the first male stop 412a, and that the upper jaw (e.g., jaw 30, jaw 78) can have a second female stop 416b (also referred to as the upper jaw first female stop 416b and other similar terms) configured to releasably engage with or releasably attach to the second male stop 412b. Half of the lower and upper jaws are shown transparent in FIGS. 8A-9B so that the shuttle 14 can be more easily seen in the jaw tracks (e.g., lower and upper tracks 66, 64), and so that the male and female stops 412, 416 can be more easily seen.

Each male stop 412 can releasably fit into, attach to, or engage with a corresponding female stop 416, for example, via a friction fit, snap fit, magnetic fit, ratchet fit, or any combination thereof. For example, the first male stop 412a can be configured to releasably attach to the first female stop 416a and the second male stop 412b can be configured to releasably attach to the second female stop 416b. When two stops (e.g., male and female stops 412, 416) are releasably attached to one another, a threshold release force can be required to release the stops from one another. The threshold release force can be from about 1.0 Newton to about 10.0 Newtons or more, including every 0.5 Newton increment within this range (e.g., 4.0 Newtons, 4.5 Newtons, 5.0 Newtons). As another example, the release force can be from about 0.5 lbs to about 1.5 lbs, including every 0.1 lb increment within this range (e.g., 1.0 lb).

The male stops 412 can be a positive feature such as a protrusion, bump, ridge, arm, extension, flexure, detent flexure, or any combination thereof. The male stops 412 can be straight and/or curved. The male stops 412 can be flexible, rigid, or both (e.g., a first portion can be flexible and a second portion can be rigid). The male stops 412 can be one or more springs. The female stops 416 can be a negative feature such as a void, space, pocket, notch, hole, through hole, recess, detent recess, or any combination thereof. The female stops 416 can be flexible, rigid, or both (e.g., a first portion can be flexible and a second portion can be rigid). The male and female stops 412, 416 can include magnets that attract one another to keep the male and female stops releasably attached together.

The male stops 412 can have a male surface 414 and the female stops 416 can have a female surface 418. The male and female surfaces 414, 418 can be configured to engage with one another, slidably engage with one another, contact one another other, or any combination thereof. The female stops 416 can have a lip 420 configured to engage with, slidably engage with, or contact the male stop 412, or any combination thereof. The male stops 412 can be configured to engage with, slidably engage with, or contact the lip 420, or any combination thereof.

For each male-female stop pair, the male and female surfaces 414,418 can engage with one another and/or the male stop 412 can engage with the lip 420, for example, when the male stop 412 is being forced into the female stop 416, when the male stop 412 is being withdrawn from the female stop 416, when the female stop 416 is being forced over or onto the male stop 412, when the female stop 416 is being withdrawn from the male stop 412, when the male and female stops 412, 416 are attached to one another (also referred to as the stopped position, caught position, fixed position), or any combination thereof. As another example, two female stops 416 can engage with one another, for example, where the two female stops 416 include a magnet. As yet another example, two male stops 412 can engage with one another, for example, where the two male stops include a magnet. The male and/or female stops 412, 416 can form a hook or hook-like feature to releasably catch the other stop.

The male stops 412 can move relative to the female stops 416, vice versa, or both. The female stops 416 can move relative to the male stops 412, vice versa, or both. For example, FIG. 8A illustrates that the male stops 412 can be translatable (e.g., slidably translatable) in the lower jaw track, for example, in a first direction toward the first female stop 416a and in a second direction away from the first female stop 416a, or vice versa such that the first female stop 416a is translatable toward and away from a male stops 412 (e.g., for arrangements where the female stop 416 is integrated with or attached to the shuttle 14 and the male stop 412 is integrated with or attached to the lower jaw). As another example, FIG. 9A illustrates that the male stops 412 can be translatable (e.g., slidably translatable) in the upper jaw track, for example, in a first direction toward the second female stop 416b and in a second direction away from the second female stop 416b, or vice versa such that the second female stop 416b is translatable toward and away from a male stop 412 (e.g., for arrangements where the female stop 416 is integrated with or attached to the shuttle 14 and the male stop 412 is integrated with or attached to the upper jaw). The first and second directions can be opposite from one another.

FIGS. 8A and 8B illustrate that when the shuttle 14 is being translated (e.g., pushed by the upper jaw pusher 86, pulled by the lower jaw pusher 76, or both) in a first direction in the lower jaw track toward the first female stop 416a, the lip 420 can exert a force against the first male stop 412a that causes the first male stop 412a to flex toward a longitudinal axis of the shuttle 14. This flexure can allow the first male stop 412a to fit into the first female stop 416a. Upon passing by the lip 420, the first male stop 412a can rebound to its neutral position or to a less flexed position and releasably lock the shuttle 14 to the lower jaw via the releasable attachment between the first male stop 412a and the first female stop 416a. FIGS. 8A and 8B further illustrate that when the shuttle 14 is being translated (e.g., pulled by the upper jaw pusher 86, pushed by the lower jaw pusher 76, or both) in a second direction (e.g., opposite the first direction) in the lower jaw track away from the first female stop 416a, the female surface 418 can exert a force against the first male stop 412a that causes the first male stop 412a to flex toward a longitudinal axis of the shuttle 14. This flexure can allow the first male stop 412a to slide under and past the lip 420. Upon passing by the lip 420 in the second direction, the first male stop 412a can rebound to its neutral position (also referred to as a non-flexed position). When the first male stop 412a flexes, it can deflect into the first suture hole 404a.

FIGS. 9A and 9B illustrate that when the shuttle 14 is being translated (e.g., pushed by the lower jaw pusher 76, pulled by the upper jaw pusher 76, or both) in a first direction in the upper jaw track toward the second female stop 416b, the lip 420 can exert a force against the second male stop 412b that causes the second male stop 412b to flex toward a longitudinal axis of the shuttle 14. This flexure can allow the second male stop 412b to fit into the second female stop 416b. Upon passing by the lip 420, the second male stop 412b can rebound to its neutral position or to a less flexed position and releasably lock the shuttle 14 to the upper jaw via the releasable attachment between the second male stop 412b and the second female stop 416b. FIGS. 9A and 9B further illustrate that when the shuttle 14 is being translated (e.g., pushed by the upper jaw pusher 86, pulled by the lower jaw pusher 76, or both) in a second direction (e.g., opposite the first direction) in the upper jaw track away from the second female stop 416b, the female surface 418 can exert a force against the second male stop 412b that causes the second male stop 412b to flex toward a longitudinal axis of the shuttle 14. This flexure can allow the second male stop 412b to slide under and past the lip 420. Upon passing by the lip 420 in the second direction, the second male stop 412b can rebound to its neutral position (also referred to as a non-flexed position). When the first male stop 412a flexes, it can deflect into the second suture hole 404b.

The lip 420 can resist passage of the first and second male stops 412a, 412b along the second direction out of the first and second female stops 416a, 416b with the threshold release force. The female surface 418 can be an inner surface of the lip 420. The lip 420 can resist passage of the first and second male stops 412a, 412b along the first direction into the first and second female stops 416a, 416b with the threshold release force or a lesser force (e.g., a force 10% to 75% of the threshold release force).

The device 188 can have zero, one, or multiple male stops 412 and zero, one, or multiple female stops 416 on the device distal end 2 (e.g., closer to the jaws than to the handle 104) and/or on the device proximal end (e.g., closer to the handle 104 than to the jaws). For example, the upper jaw (e.g., upper jaw 78) can have one or multiple male stops 412, one or multiple female stops 416, or any combination thereof. The lower jaw (e.g., lower jaw 80) can have one or multiple male stops 412, one or multiple female stops 416, or any combination thereof. The male and/or female stops 412 and/or 416 can be attached to or integrated with the jaw, the jaw track, or both. The shuttle 14 can have one or multiple male stops 412, one or multiple female stops 416, or any combination thereof. The male stops 412 can extend away from and/or toward a longitudinal axis of the shuttle 14. The male stops 412 can extend away from and/or toward a longitudinal axis of a jaw track (e.g., tracks 66 and 64). The female stops 416 can extend away from and/or toward a longitudinal axis of the shuttle 14. The female stops 416 can extend away from and/or toward a longitudinal axis of a jaw track (e.g., tracks 66 and 64).

For example, FIG. 8A illustrates that the shuttle 14 can have a first male stop 412a and a second male stop 412b, and that the lower and upper jaw tracks (e.g., tracks 66 and 64) can each define a female stop 416 (e.g., a first female stop 416a in the lower jaw and a second female stop 416b in the upper jaw). The lower and upper jaws can each define one or multiple female stops 416. For example, the first male stop 412a can releasably attach to the first female stop 416a and the second male stop 412b can releasably attach to a second female stop 416b. FIG. 8A illustrates that the first and second male stops 412a, 412b can extend away from a longitudinal axis (e.g., center longitudinal axis) of the shuttle toward a longitudinal center of the shuttle 14. The first and second male stops 412a, 412b can be the same or a different dimension away from the longitudinal center of the shuttle 14 as the other detent.

FIGS. 8A-9B further illustrate a surface 422 of jaw suture slots 238, for example, lower jaw suture slot 238a and upper jaw suture slot 238b.

FIGS. 8B and 9B illustrate that the shuttle tips 164 can have a shuttle tip thickness 408 of about 0.05 mm to about 0.75 mm, including every 0.05 mm increment within this range (e.g., 0.15 mm, 0.20 mm, 0.25 mm). The shuttle tip thickness 408 can be the width of the non-beveled surfaces 406. As another example, the shuttle tip thickness 408 can be from about 0.0080 in. to about 0.0090 in. (e.g., 0.0085 in.).

FIGS. 8B and 9B further illustrate that the shuttle 14 can have a shuttle thickness 410 (also referred to as the shuttle thickness 14_{T}) of about 0.05 mm to about 0.75 mm, including every 0.05 mm increment within this range (e.g., 0.15 mm, 0.20 mm, 0.25 mm). The shuttle tip thickness 408 can be the same or different from the shuttle thickness 410. The shuttle tip thickness 408 can be less than, equal to, or greater than the shuttle thickness 410. For example, the shuttle tip thickness 408 can be about 0.15 mm and the shuttle thickness 410 can be about 0.25 mm, or vice versa.

The shuttle 14 can be made from a single panel of material (e.g., metal). The suture holes 404 can be cut, leaving the shuttle 14 and the male stops 412. The shuttle 14 can then be bent, which can result in the male stops 412 extending out of the plane of the shuttle spine 160.

FIGS. 8A-9B further illustrate that a portion 423 of the shuttle tips 164 can remain exposed outside of the jaws when the shuttle 14 is fully translated into the jaws. The exposed portion 423 can have an exposed length 424, for example, from about 0.25 mm to about 5.00 mm or more, including every 0.25 mm increment within this range (e.g., 0.50 mm, 1.00 mm, 1.50 mm). The exposed portion 423 can align the lower and upper jaws when they close. The exposed portion 423 can pierce tissue when the lower and upper jaws are closed against each other and before the shuttle 14 is translated to the other jaw. This can advantageously leverage the clamping force of the jaws to cut tissue with the shuttle 14. A portion of the exposed portion 423 can pierce tissue when the shuttle 14 is translated from the lower jaw to the upper jaw, or vice versa.

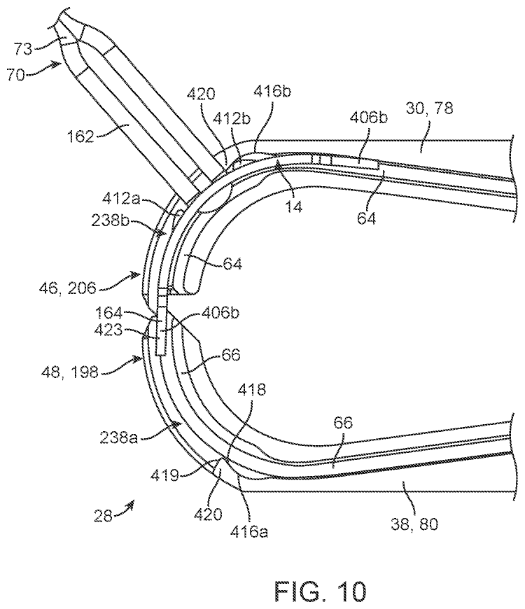

FIG. 10 illustrates that all or a portion of the exposed portion 423 can be in the other jaw when the lower and upper jaws are closed and before the shuttle 14 is translated to the other jaw via the lower or upper pusher 76, 86. For example, when the jaws are moved from an open configuration to a closed configuration with the exposed portion 423 extending from the upper jaw (e.g., jaw 30, jaw 78) as shown in FIGS. 8A and 8B, the exposed portion 423 can be moved into the lower jaw (e.g., jaw 38, jaw 80) via the jaws closing with or without translation (e.g., simultaneous translation) of the shuttle 14 into the lower jaw via an upper and/or lower pusher while the jaws are being closed.

FIG. 10 illustrates that the female stops 416 can have an outer surface 419 and an inner surface 418 (also referred to as a female surface). The outer surface 419 can be flat or curved. The outer surface 419 can define a ramp surface for the male stops 412 to flex against. The outer surface 419 can define a plane at an angle to a longitudinal axis of the shuttle. For example, the plane of the outer surface 419 can be perpendicular or substantially perpendicular to the center longitudinal axis of the shuttle. The inner surface can be flat or curved. The inner surface 418 can define a ramp surface for the male stops 412 to flex against.

FIG. 10 illustrates that when the lower and upper jaws are closed the jaws can define a continuous track for the shuttle 14 such the lower jaw track 66 and the upper jaw track 64 are continuous with one another. The tracks of the upper jaw and bottom jaw can form a continuous path when the jaw structure 28 is in a closed configuration.

FIG. 10 illustrates that the first jaw tip (e.g., jaw tip 46, jaw tip 206) can be configured to interdigitate with the second jaw tip (e.g., jaw tip 48, jaw tip 198). For example, the first jaw tip can interdigitate with and be adjacent or in contact with the second jaw tip when the jaw structure 28 is in a closed configuration. The jaw tips can be sharpened. The jaw tips can be tapered. The jaw tips can be sharp and seat into each other to form a continuous track when the jaw structure 28 is in a closed configuration. The jaw tips can seat into each other to connect the lower and upper jaw tracks 66, 64 to each other.

FIG. 11A illustrates that the shuttle 14 can have a shuttle first tip 164a and a shuttle second tip 164b.

FIG. 11A illustrates that the shuttle 14 can have zero, one, or multiple shuttle holes 405, for example, 1 to 6 or more shuttle holes 405, including every 1 shuttle hole increment within this range (e.g., 2 shuttle holes, 4 shuttle holes). The shuttle 14 can have a first shuttle hole 405a and a second shuttle hole 405b. The shuttle holes 405 (e.g., holes 405a and 405b) can be the same as or different from the suture holes 404 (e.g., holes 404a and 404b). The male and/or female stops 412, 416 can move into and out of the shuttle holes 405, the suture holes 404, or any combination thereof, for example, via flexing, bending, translating, and/or rotating into and out of the holes 405 and/or 404.

FIG. 11A illustrates that the male stops 412 can have one or multiple bends 426. For example, FIG. 11A illustrates that the male stops 412 can have a first bend 426a and a second bend 426b. The male stops 412 can have inflection points 426_{IF} where the curvature of the male stop 412 changes direction or its concavity. For example, FIG. 11A illustrates that the male stops 412 can have an inflection point 426_{IF} between two bends 426 (e.g., between the first and second bends 426a, 426b) where the male stops 412 change concavity (e.g., from concave up for the first bend 426a to concave down for the second bend 426b as shown in FIG. 11A).

FIG. 11A illustrates that one or more magnets 428 can be attached to or integrated with the male stops 412 on a first side of the detents, for example, on or under the first surface (also referred to as a male surface). The magnets 428 can be configured to be magnetically attracted to a magnet attached to or integrated with the upper or lower jaw having an opposite dipole as the magnets 428.

FIG. 11B illustrates that the one or more magnets 428 can be attached to or integrated with a second surface 415 of the male stops 412, for example, to an underside of the male stops 412.

FIG. 11B illustrates that there can be a gap G on each side of the male stop 412 between the male stop 412 and the shuttle body 160. The gap G can advantageously inhibit or prevent pressure from forming in the jaws by allowing gas, liquid, or solids to flow or pass through the gap G as the shuttle 14 is advanced into the jaws. There can be a gap on each lateral side of the male stop 412 as shown in FIG. 11B. As another example, there may not be any gaps G between the male stops 412 and the shuttle body 160, or the gap G can be on only one side of the male stop 412 instead of both sides as shown in FIG. 11B. The gaps G can have a gap width G_{w} that can be, for example, constant (e.g., as shown in FIG. 11B) or tapered. As another example, the gaps G can have multiple gap widths G_{w}. For example, FIG. 11B illustrates that the gap G can have a constant width, for example, as measured between the lateral edge or surface of the male stop 412 and the lateral edge or surface of the shuttle body 160.

FIG. 11B illustrates that the terminal tips 165 can have a sharpened edge. The sharpened edge can be configured to pierce tissue.

FIG. 11C illustrates that the male stops 412 (e.g., first and second male stops 412a, 412b) can extend away from a longitudinal axis of the shuttle 14 out of the plane of the shuttle spine 160.

The shuttle longitudinal axis (e.g., longitudinal axis 157) can be flat or curved. FIG. 11C illustrates that the shuttle radius of curvature 154 can be from about 2.00 mm to about 5.00 mm or more, including every 0.01 mm increment within this range (e.g., 2.84 mm).

The shuttle 14 can be straight or have a preformed bend or curve (e.g., having the radius of curvature 154). The shuttle 14 can have a preformed bend having a radius of curvature of about 40% to about 200% of the radius of curvature 154, including every 1% increment within this range (e.g., 50%).

The curvature of the shuttle 14 can be constant. The curvature of the shuttle 14 can be fixed. The shuttle 14 can be flexible. The shuttle 14 can be rigid. The shuttle 14 can transition between curved and straight configurations. Having a preformed bend within this range can reduce the strain on the shuttle 14. For example, the strain can be reduced for variations where the shuttle 14 shifts between straight and curved configurations when moving within and/or between the jaws.

FIGS. 11D and 11E illustrate that the male stops 412 (e.g., the first and second males stops 412a, 412b) can be radial male stops. FIGS. 11D and 11E illustrate that the male stops 412 (e.g., the first and second males stops 412a, 412b) can extend radially away from the shuttle 14. FIGS. 11D and 11E illustrate that the male stops 412 (e.g., the first and second males stops 412a, 412b) can have a hammerhead shape such that a first end of the males stops can be smaller (e.g., narrower) than a second end of the male stops 412. FIGS. 11D and 11E illustrate that the male stops 412 (e.g., the first and second male stops 412a, 412b) can be deflectable toward and away a longitudinal axis of the shuttle 14. For example, FIGS. 11D and 11E illustrate that the male stops 412 (e.g., the first and second male stops 412a, 412b) can be deflectable toward and away from the openings in the shuttle 14 shown in FIGS. 11D and 11E.

FIGS. 11F and 11G illustrate that the male stops 412 (e.g., the first and second males stops 412a, 412b) can be lateral male stops. FIGS. 11F and 11G illustrate that the male stops 412 (e.g., the first and second males stops 412a, 412b) can extend laterally away from the shuttle 14. FIGS. 11F and 11G illustrate that the male stops 412 (e.g., the first and second males stops 412a, 412b) can have a hammerhead shape such that a first end of the males stops 412 can be smaller (e.g., narrower) than a second end of the male stops 412. FIGS. 11F and 11G illustrate that the male stops 412 (e.g., the first and second male stops 412a, 412b) can be deflectable toward and away from a longitudinal axis of the shuttle 14. For example, FIGS. 11F and 11G illustrate that the male stops 412 (e.g., the first and second male stops 412a, 412b) can be deflectable toward and away from the openings in the shuttle 14 shown in FIGS. 11F and 11G.

The shuttle 14 can have any combination of the male stops 412 shown in FIGS. 11A-11G. For example, the shuttle 14 can have 1, 2, 3, or 4 male stops 412. The shuttle 14 can have any combination of male stops 412. For example, the shuttle 14 can have any combination of radial male stops (e.g., the male stops 412 shown in FIGS. 11A-E) and lateral male stops (e.g., the male stops 412 shown in FIGS. 11F and 11G), including, for example, two radial male stops (e.g., two of the male stops 412 shown in FIGS. 11A-11E), two lateral male stops (e.g., the male stops 412 shown in FIGS. 11F and 11G), one radial male stop on a first end of the shuttle 14 and one lateral male stop on a second end of the shuttle 14 (e.g., one of the male stops 412 shown in FIGS. 11A-11E and one of the male stops 412 shown in FIGS. 11F and 11G), and two radial male stops and two lateral male stops for a total of four male stops 412 (e.g., two of the male stops 412 shown in FIGS. 11A-11E and the male stops 412 shown in FIGS. 11F and 11G). As another example, the shuttle 14 may not have any male stops 412.

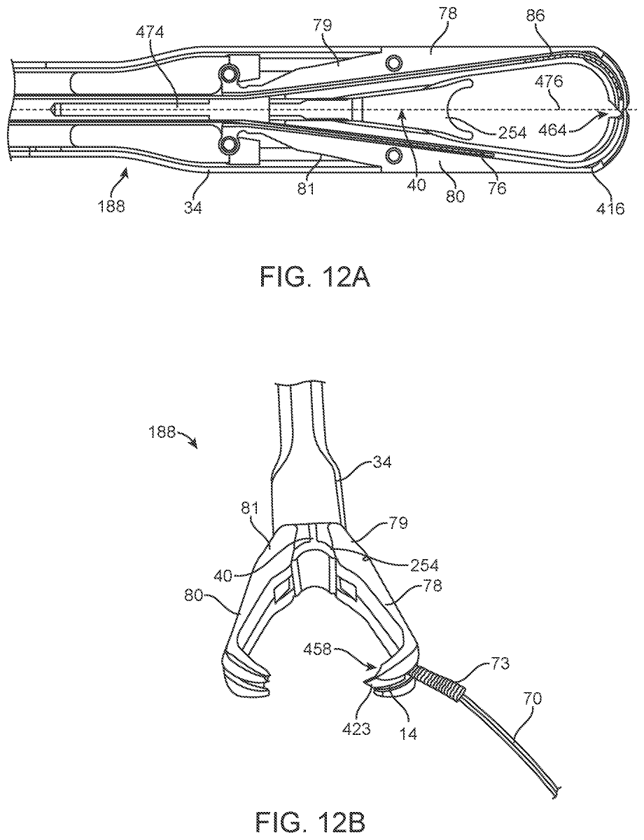

FIGS. 12A and 12B illustrate a variation of the device 188 in a fully closed and fully open configuration, respectively.

FIGS. 12A and 12B illustrate that the jaw control extension 40 can be fixed and that the jaws 78 and 80 can move relative to the jaw control extension 40. For example, the jaws 78 and 80 can move distally and proximally against the jaw control extension 40 to open and close, respectively. The jaws 78 and 80 can move longitudinally along a device longitudinal axis 476. The jaws 78 and 80 can move into and out of the compression cover 34. The jaws 78 and 80 can be attached to a tube 474 connected to the handle controls that can translate (e.g., slidably translate) the jaws 78 and 80 into and out of the compression cover 34. FIGS. 12A and 12B illustrate that the compression cover 34 can engage with an upper jaw surface 79 and a lower jaw surface 81 to force the jaws closed when the jaws 78 and 80 are translated proximally toward the handle and into the compression cover 34. Movement of the jaws 78 and 80 in a first direction (e.g., distal movement) against the jaw control extension 40 can cause the jaws to open and move from the closed configuration shown in FIG. 12A to the open configuration shown in FIG. 12B. Movement of the jaws 78 and 80 in a second direction (e.g., proximal movement) against the compression cover 34 can cause the jaws to close and move from the open configuration shown in FIG. 12B to the closed configuration shown in FIG. 12A. The first and second directions can be opposite from one another. As another example, the jaws can be fixed and the jaw control extension can move relative to the jaws. The compression cover 34 can be longitudinally fixed or longitudinally movable.

The extension head 254 can have the shape shown such that the jaws open relative to each other when the jaws are moved out of the compression cover 34 over the extension head 254.

FIG. 12A illustrates the device 188 without a shuttle 14 for illustrative purposes and FIG. 12B illustrates the device 188 of FIG. 12A with a shuttle 14.

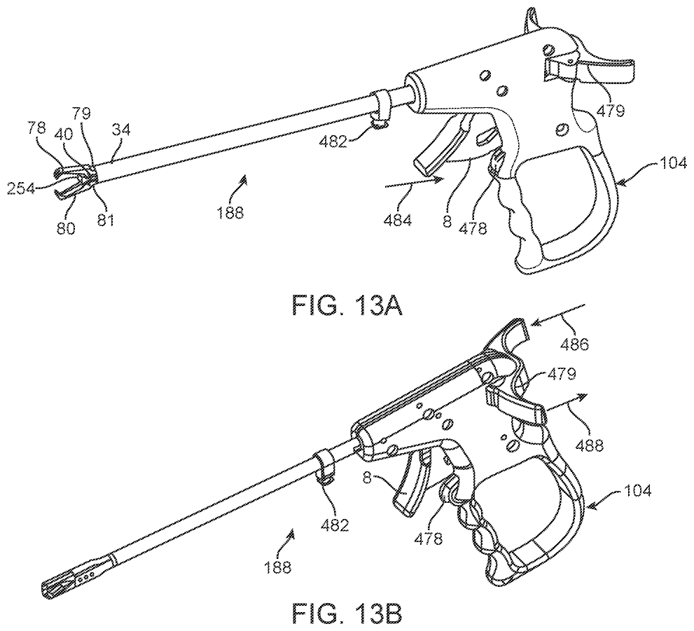

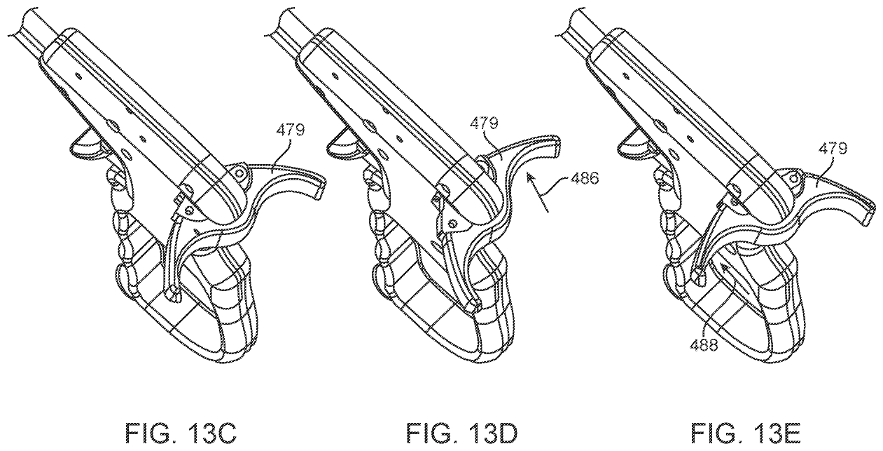

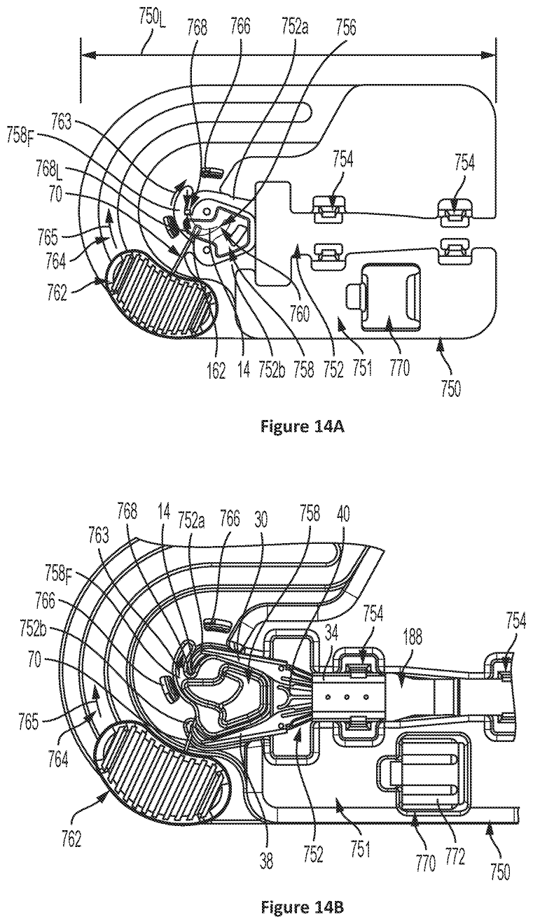

FIGS. 13A and 13B illustrate a variation of the device 188 in a fully opened and fully closed configuration, respectively.

FIGS. 13A and 13B illustrate that the handle 104 can have a jaw control 8 (also referred to as a trigger), a jaw control release 478 and a shuttle control 479. The jaw control 8 can be pulled with one or more fingers in direction 484 to move the jaw control 8 to the configuration shown in FIG. 13B. The jaw control 8 can translate and/or rotate. When the jaw control 8 is moved in direction 484, the jaws 78 and 80 can move from an open configuration to a less open configuration (e.g., to the closed configuration shown in FIG. 13B). When the jaw control 8 is moved in a direction opposite to direction 484, the jaws 78 and 80 can move from a closed configuration to an open configuration (e.g., from the closed configuration in FIG. 13B to the fully open configuration illustrated in FIG. 13A).

As another example, the handle 104 can have a first press button configured to close the jaws when pressed and a second press button configured to open the jaws when pressed.

The jaw control release 478 can be a press button, a switch, a knob, or any combination thereof. The jaw control 8 can lock when the jaws 78 and 80 are in the fully closed configuration. Activating the jaw control release 478 can release the jaw control 8 from the lock position. The jaw control release 478 can be activated, for example, by pressing it. Upon pressing the jaw control release 478, the jaw control 8 can be manually returned to the position shown in FIG. 13A to fully open the jaws, or the jaw control 8 can automatically return to the position shown in FIG. 13A.

The shuttle control 479 can be a button, switch, knob, or any combination thereof. For example, FIGS. 13A and 13B illustrate that the shuttle control 479 can be a switch that can pivot. The shuttle control 479 can be locked when the jaws 78 and 80 are in the open configuration of FIG. 13A. When the jaws are closed, the shuttle control can be rotated in direction 486 and direction 488. Directions 486 and 488 can be directed opposite from one another. When the shuttle control 479 is moved in (e.g., rotated) in direction 486, the upper pusher 86 can move the shuttle 14 to the lower jaw 80. When the shuttle control 479 is moved in (e.g., rotated) in direction 488, the lower pusher 76 can move the shuttle 14 to the upper jaw 78. The shuttle control 479 can have a batwing shape, which can provide ergonomic benefits.

As another example, the handle 104 can have a first press button configured to move the upper pusher 86 when pressed and a second press button configured to move the lower pusher 76 when pressed.

FIGS. 13A and 13B further illustrate that the device 188 can have a flush port 482 having a luer connection. A cleaning fluid (e.g., enzyme cleaner) can be flushed through the device through the flush port 482 to clean it.

FIG. 13C illustrates that the shuttle control 479 can have a neutral position. When the shuttle control 479 is in the neutral position, one or neither of the pushers 76 and 86 can be in contact with the shuttle 14. When the shuttle control 479 is in the neutral position, the shuttle 14 can be ejected from the jaws.

FIG. 13D illustrates the shuttle control 479 in a fully advanced position when moved in direction 486. When in the shuttle control 479 is in the fully advanced position in direction 486, the upper pusher 86 can be in a fully advanced position and the lower pusher 76 can be in a fully retracted position. For example, the upper pusher 86 can be fully advanced toward the lower jaw 80, thereby pushing the shuttle 14 into the lower jaw 80. The upper pusher 86 can push the shuttle 14 into the lower jaw 80 to the point where detents on the shuttle 14 (male and/or female stops 412, 416) releasably engage with detents on the lower jaw 80 (male and/or female stops 412, 416).

FIG. 13E illustrates the shuttle control 479 in a fully advanced position when moved in direction 488. When in the shuttle control 479 is in the fully advanced position in direction 488, the lower pusher 76 can be in a fully advanced position and the upper pusher 86 can be in a fully retracted position. For example, the lower pusher 76 can be fully advanced toward the upper jaw 78, thereby pushing the shuttle 14 into the upper jaw 78. The lower pusher 76 can push the shuttle 14 into the upper jaw 78 to the point where detents on the shuttle 14 (male and/or female stops 412, 416) releasably engage with detents on the upper jaw 78 (male and/or female stops 412, 416).

The upper and lower jaws referred to throughout the application can be any of the upper and lower jaws disclosed, illustrated, and/or contemplated herein. For example, the upper and lower jaws 30, 48 can be the upper and lower jaws 78, 80, respectively. As another example, the upper jaw 30 can be interchangeable with the upper jaw 78, and the lower jaw 38 can be interchangeable with the upper jaw 80. As yet another example, the upper jaw 30 can also be referred to as the upper jaw 78, and the lower jaw 38 can also be referred to as the lower jaw 80. The lower jaw can be a first jaw and the upper jaw can be a second jaw. The lower jaw can be a second jaw and the upper jaw can be a first jaw.

The upper and lower jaw tracks referred to throughout the application can be any of the upper and lower jaw tracks disclosed, illustrated, and/or contemplated herein. For example, the upper and lower jaw tracks 64, 66 can be the upper and lower jaw tracks 264, 148, respectively. As another example, the upper jaw track 64 can be interchangeable with the upper jaw track 264, and the lower jaw track 66 can be interchangeable with the upper jaw track 264. As yet another example, the upper jaw track 64 can also be referred to as the upper jaw track 264, and the lower jaw track 66 can also be referred to as the lower jaw track 148. The lower jaw track can also be referred to as the lower track, and the upper jaw track can also be referred to as the upper track.

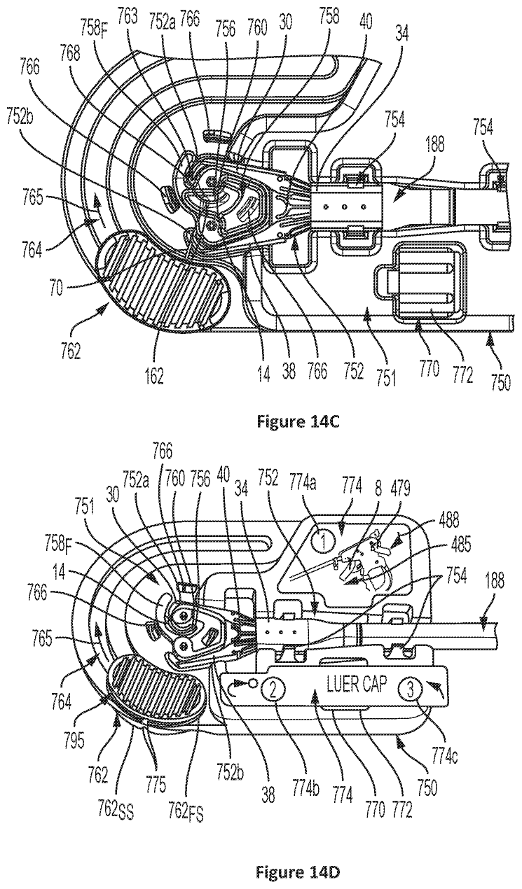

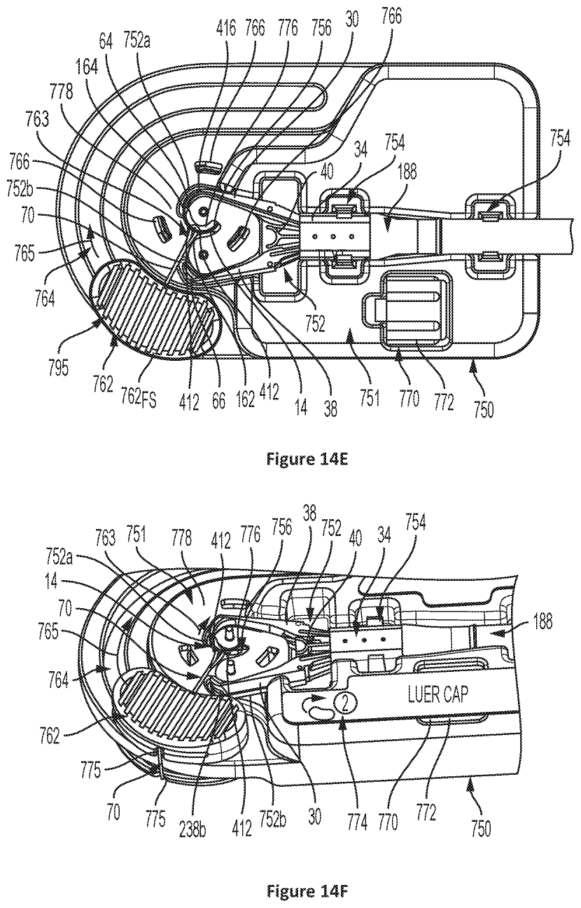

FIG. 14A illustrates a variation of a shuttle loader 750 (also referred to as a loader) that can load the shuttle 14 into the device 188, for example, into the upper jaw 30 or into the lower jaw 38. The loader 750 can have a loader body 751 that can have a device space 752. The device 188 can be removably positionable in the device space 752. When the device 188 is in the device space 752, the shuttle 14 can be loaded into the device 188. The device space 752 can be one or multiple spaces. For example, FIG. 14A illustrates that the device space 752 can be a recess in the loader body 751. The spaces can be recesses, grooves, depressions, or available space on the loader 750. FIG. 14A illustrates that the device space 752 can include a first jaw space 752a and a second jaw space 752b. For example, FIG. 14A illustrates that the first jaw space 752a can be a recess and that the second jaw space 752b can be a recess. The upper jaw 30 jaw can be placed in the first jaw space 752a and the lower jaw 38 can be placed in the second jaw space 752b. As another example, the lower jaw 38 jaw can be placed in the first jaw space 752a and the upper jaw 30 can be placed in the second jaw space 752b. The device 188 can be placed in the device space 752 in a partially open configuration or a fully open configuration. For example, FIG. 14A illustrates that the upper and lower jaws 30, 38 can be positionable in the first and second jaw spaces 752a, 752b when the jaws 30 and 38 are in a fully open configuration.

FIG. 14A illustrates that the loader 750 can have one or multiple holders 754 that can hold the device 188 and the loader 750 together. For example, FIG. 14A illustrates that the holders 754 can hold the device 188 in the device space 752. The holders 754 can be, for example, clips, clasps, magnets, or fasteners, clasps, catches, pins, or any combination thereof. For example, FIG. 14A illustrates that the holders 754 can be clips that the device 188 can be snapped into when the device 188 is placed in the device space 752. As another example, the device space 752 can be sized and shaped to form an interference fit (also referred to as a friction fit) with the device 188 when the device 188 is in the device space 752. For example, the walls of the device space 752, the holders 754, or the walls of the device space 752 and the holders 754 can form an interference fit with the device 188 when the device 188 is in the device space 752.

FIG. 14A illustrates that the loader 750 can have a loader body shuttle track 756 (also referred to as the shuttle track 756 or the track 756). The track 756 can be a track in the loader body 751. The track 756 can be a groove in the loader body 751. The track 756 can be a channel in the loader body 751. The track 756 can have the same radius of curvature as the shuttle 14. The shuttle 14 can be in the shuttle track 756 or can be positionable in the shuttle track 756. For example, FIG. 14A illustrates that the loader 750 can be packaged with the shuttle 14 positioned in the shuttle track 756 in the arrangement shown.

The shuttle 14 can be moveable (e.g., translatable, slidable) in the track 756. The shuttle 14 can be moveable (e.g., longitudinally movable) along the track 756. For example, the shuttle 14 can be translatable or slidable along the track 756. The shuttle 14 can be moveable from the track 756 into the device 188. For example, the shuttle 14 can be moveable out of (e.g., longitudinally out of) the track 756, for example, into the upper jaw track 64 or into the lower jaw track 66. The shuttle 14 can be moveable (e.g., translatable, slidable) from the track 756 into the device 188, into the first jaw space 752a, into a space above the first jaw space 752a, or into any combination thereof. The shuttle 14 can be pushable along the track 756, pullable along the track 756, or both. The shuttle 14 can be pushable out of the track 756, pullable out of the track 756, or both. When the device 188 is attached to the loader 750, for example, as shown in FIG. 14B, the shuttle 14 can be movable into whichever jaw (e.g., the upper jaw 30 or the lower jaw 38) is in the first jaw space 752a. When the upper jaw 30 is in the first jaw space 752a, the shuttle 14 can be moveable from the track 756 to the upper jaw track 64, for example, by pushing and/or pulling the shuttle 14 from the track 756 to the upper jaw track 64. When the lower jaw 38 is in the first jaw space 752a, the shuttle 14 can be moveable from the track 756 to the lower jaw track 66, for example, by pushing and/or pulling the shuttle 14 from the track 756 to the lower jaw track 66. FIGS. 14A and 14B illustrate that the loader 750 can have a loader control 762 that can move the shuttle 14. The loader control 762 can be movable by the user to load the shuttle 14 from the track 756 into the device 188. The loader control 762 can be pushable and/or pullable to load the shuttle 14 into the device 188. For example, FIGS. 14A and 14B illustrate that the loader control 762 can be moved to pull the shuttle 14 into the device 188. FIGS. 14A and 14B illustrate that the shuttle 14 and the loader control 762 can be releasably connected, for example, via the suture 70.

The shuttle 14 can have a shuttle first position and a shuttle second position. The shuttle first position can be a non-loaded position of the shuttle 14. The shuttle second position can be a loaded position of the shuttle 14. The shuttle 14 can be moveable from the shuttle first position to the shuttle second position. The shuttle 14 can be moved from the shuttle first position to the shuttle second position, for example, in a first direction 763 along the track 756. The shuttle 14 can be moved from the shuttle first position to the shuttle second position to load the device 188 with the shuttle 14. The shuttle 14 can be moved from the shuttle first position to the shuttle second position to load the shuttle 14 into the device 188. When the shuttle 14 is in the shuttle first position, the shuttle 14 can be in the track 756. When the shuttle 14 is in the shuttle first position, the shuttle 14 can be between the first jaw space 752a and the second jaw space 752b. When the shuttle 14 is in the shuttle second position, the shuttle 14 can be in the upper jaw 30 or in the lower jaw 38, for example, in the upper jaw track 64 or in the lower jaw track 66. When the shuttle 14 is in the shuttle second position, the shuttle 14 can be in or above the first jaw space 752a. For example, FIG. 14A illustrates that the shuttle first position can be the position of the shuttle 14 when the shuttle 14 is in the track 756. The shuttle first position can be the home position (also referred to as the neutral position) of the shuttle 14. The shuttle first position can be the position of the shuttle 14 before the shuttle 14 is loaded into the device 188. The loader 750 can be packaged with the shuttle 14 in the shuttle first position. As another example, the user can place the shuttle 14 into the shuttle first position. The shuttle second position can be a shuttle loaded position (e.g., a fully loaded position, a partially loaded position) of the shuttle 14 in the device 188.

A fully loaded position of the shuttle 14 can be the position of the shuttle 14 when, for example, the shuttle 14 is in the device 188 (e.g., is in the upper jaw 30 or in the lower jaw 38) and a male stop 412 is engaged with another male stop 412 or with a female stop 416. If the device 188 does not have any stops, the fully loaded position of the shuttle 14 can be, for example, when the shuttle 14 is in the device 188 (e.g., is in the upper jaw 30 or in the lower jaw 38) to such an extent that the upper and lower jaws 30, 38 can be closed (e.g., fully closed) with the shuttle 14 in the device 188.

A partially loaded position of the shuttle 14 can be any position of the shuttle 14 between the shuttle first position and a fully loaded position of the shuttle 14. For example, a partially loaded position of the shuttle 14 can be when the shuttle 14 is in the device 188 (e.g., is in the upper jaw 30 or in the lower jaw 38) but before a male stop is engaged with another male stop 412 or with a female stop 416. If the device 188 does not have any stops, a partially loaded position of the shuttle 14 can be, for example, when the shuttle 14 is in the device 188 (e.g., is in the upper jaw 30 or in the lower jaw 38) but the upper and lower jaws 30, 38 cannot be closed with the shuttle 14 in the device 188 (e.g., because the shuttle 14 is inhibiting or preventing the jaws 30 and 38 from being closed), or the jaws 30 and 38 can be partially closed but the shuttle 14 prevents the jaws 30 and 38 from being fully closed.

FIG. 14A illustrates that the loader 750 can have the suture 70, the suture loop 162, or both. The suture 70 can be attached to the shuttle 14 or can be attachable to the shuttle 14 with or without the suture loop 162. For example, FIG. 14A illustrates that the loader 750 can be packaged with the suture 70 and the suture loop 162 in the arrangement shown, with the suture loop 162 attached to the shuttle 14.

FIG. 14A illustrates that the loader 750 can have a cap 758. The cap 758 can be opaque or transparent. For example, FIG. 14A illustrates that the cap 758 can be transparent. A transparent cap 758 can advantageously allow the user to observe the loading process, for example, so that the user can see whether or not the shuttle 14 is properly loading into the device 188 during the loading process. A transparent cap 758 can advantageously allow the user to inspect the shuttle 14 prior to loading the shuttle 14 into the device 188. The cap 758 can keep the shuttle 14 in the shuttle track 756. The cap 758 can have a cap shuttle track 760 (also referred to as the shuttle track 760 or the track 760) that can mate with the shuttle track 756. A first lateral side of the shuttle 14 can be in the track 756 and a second lateral side of the shuttle 14 can be in the track 760. The cap 758 can be attached to the loader body 751, for example, with glue. As another example, the cap 758 can be removably attached to the loader body 751. The cap 758 can define a portion of the device space 752. The cap 758 can have a finger 758_{F} (also referred to as an extension). The finger 758_{F} can constrain or guide movement of the shuttle 14 in the first jaw space 752a. The finger 758_{F} can define a side wall of the first jaw space 752a. The finger 758_{F} can constrain or guide the device 188 when in the first jaw space 752a. The finger 758_{F} can extend around an end (e.g., distal end) of the first jaw space 758a. As another example, the cap 758 may not have the finger 758_{F}.

FIG. 14A illustrates that the loader 750 can have a loader control track 764 (also referred to as the track 764) for the loader control 762. The loader control 762 can be, for example, a moveable button, a moveable knob, a moveable toggle, a moveable switch, a moveable slide, a translator, a rotator, a slider, or any combination thereof. The loader control 762 can have an ergonomic shape, for example, the shape shown in FIG. 14A. For example, FIG. 14A illustrates that the loader control 762 can have a bean shape (e.g., a kidney bean shape) when viewed from a top view. The loader control 762 can be translatable and/or rotatable. For example, FIG. 14A illustrates that the loader control 762 can be a moveable button. The track 764 can be, for example, a track, a channel, a groove, or a through channel of the loader body 751, or any combination thereof. For example, FIG. 14A illustrates that the track 764 can extend through the loader body 751. For example, FIG. 14A illustrates that the track 764 can be a slot.

The loader control 762 can be moveable (e.g., translatable, slidable) in and/or along the loader control track 764. The loader control 762 can be moved back and forth along the track 764. The loader control 762 can have a loader control first position and a loader control second position. The loader control 762 can be moveable from the loader control first position to the loader control second position. The loader control 762 can be moved from the loader control first position to the loader control second position, for example, in a first direction 765 along the track 764. The loader control first position can be at a first end of the track 764. The loader control second position can be at the first end of the track 764, or at a second end of the track 764. For example, FIG. 14A illustrates that the loader control first position can be at a first terminal end of the track 764. The loader control first position can be the home position (also referred to as the neutral position or a non-loaded position) of the loader control 762. The loader 750 can be packaged with the loader control 762 in the loader control first position, with the suture loop 162 attached to the shuttle 14. The loader control second position can be at a second terminal end of the track 764 or any position along the track 764 between the first and second terminal ends of the track 764. The loader control second position can be a loader control loaded position (e.g., a fully loaded position, a partially loaded position). For example, the loader control first position can be a non-loaded position of the loader control 762, the loader control positions between the loader control first and second positions can be loader control partially loaded positions of the loader control 762, the loader control second position can be a fully loaded position of the loader control 762, and the loader control positions beyond the loader control second position (e.g., along the track 764 in direction 765) can be loader control over-loaded positions of the loader control 762. The loader control first position can be the position of the loader control 762 when the shuttle 14 is in the shuttle first position (e.g., a non-loaded position). The loader control second position can be the position of the loader control 762 when the shuttle 14 is in the shuttle second position (e.g., a fully loaded position). The loader control first position can be the position of the loader control 762 before the shuttle 14 is loaded into the device 188. The loader control second position can be the position of the loader control 762 after the shuttle 14 is loaded into the device 188.

A fully loaded position of the loader control 762 can be the position of the loader control 762 when, for example, the shuttle 14 is in the device 188 (e.g., is in the upper jaw 30 or in the lower jaw 38) and a male stop 412 is engaged with another male stop 412 or with a female stop 416. If the device 188 does not have any stops, the fully loaded position of the loader control 762 can be, for example, when the shuttle 14 is in device 188 (e.g., is in the upper jaw 30 or in the lower jaw 38) to such an extent that the upper and lower jaws 30, 38 can be closed (e.g., fully closed) with the shuttle 14 in the device 188.

A partially loaded position of the loader control 762 can be any position of the loader control 762 between the loader control first position and a fully loaded position of the loader control 762. For example, a partially loaded position of the loader control 762 can be when the shuttle 14 is in the device 188 (e.g., is in the upper jaw 30 or in the lower jaw 38) but before a male stop is engaged with another male stop 412 or with a female stop 416. If the device 188 does not have any stops, a partially loaded position of the loader control 762 can be, for example, when the shuttle 14 is in the device 188 (e.g., is in the upper jaw 30 or in the lower jaw 38) but the upper and lower jaws 30, 38 cannot be closed with the shuttle 14 in the device 188 (e.g., because the shuttle 14 is inhibiting or preventing the jaws 30 and 38 from being closed), or the jaws 30 and 38 can be partially closed but the shuttle 14 prevents the jaws 30 and 38 from being fully closed.

The loader control 762 can be moveable from the loader control first position to the loader control second position to move the shuttle 14 into the device 188, for example, to move the shuttle 14 from the shuttle first position to the shuttle second position. To load the shuttle 14 into the device 188, the loader control 762 can be moved (e.g., pushed, pulled, pushed and pulled) from the loader control first position to the loader control second position or to a loader control third position beyond the loader control second position. The loader control third position can be any position beyond the loader control second position, and can include, for example, the position of the loader control 762 at the second terminal end of the track 764.

When the loader control 762 is in the loader control first position, the shuttle 14 can be in the shuttle first position. When the loader control 762 is in the loader control second position, the shuttle 14 can be in the shuttle second position. The shuttle second position can be a loaded position of the shuttle 14 in the device. When the loader control 762 is in the loader control third position, the shuttle 14 can be in the shuttle second position or in a shuttle third position. The shuttle third position can be an overloaded position of the shuttle 14 in the device 188. The shuttle second and third positions can both be fully loaded positions of the shuttle 14. For example, one of the male stops 412 (e.g., the leading male stop 412, which can be the male stop 412 that is loaded the farthest into the device 188 when the shuttle 14 is in the shuttle second and third positions) can be engaged with another male stop 412 or with a female stop 416 when the shuttle 14 is in the shuttle second position and when the shuttle 14 is in the shuttle third position. The user can choose to load the shuttle 14 to the shuttle second position or to the shuttle third position. The device 188 can be considered loaded with the shuttle 14 when the shuttle 14 is in the shuttle second position. The device 188 can be considered loaded with the shuttle 14 when the shuttle 14 is in the shuttle third position. For example, the device 188 can be removed from the loader 750 when the device 188 is in a device loaded configuration. The device 188 can have the device loaded configuration when the shuttle 14 is in the shuttle second position or when the shuttle 14 is in the shuttle third position.

When the device 188 is in the device loaded configuration and the device 188 is detached from the loader 750, the upper and lower jaws 30, 38 can be closed, and the shuttle 14 can be passed from the upper jaw 30 to the lower jaw 38 or vice versa.

For example, when the device 188 is in the device loaded configuration with the shuttle in the shuttle second position in the upper jaw 30, the device 188 can detached from the loader 750, the upper and lower jaws 30, 38 can be closed, and the shuttle 14 can be passed from the upper jaw 30 to the lower jaw 38, for example, by moving (e.g., pushing) the shuttle 14 from the shuttle second position in the upper jaw 30 to a position in the lower jaw 38.

For example, when the device 188 is in the device loaded configuration with the shuttle in the shuttle third position in the upper jaw 30, the device 188 can detached from the loader 750, the upper and lower jaws 30, 38 can be closed, and the shuttle 14 can be passed from the upper jaw 30 to the lower jaw 38, for example, by moving (e.g., pushing) the shuttle 14 from the shuttle third position in the upper jaw 30 to a position in the lower jaw 38.

For example, when the device 188 is in the device loaded configuration with the shuttle in the shuttle second position in the lower jaw 38, the device 188 can detached from the loader 750, the upper and lower jaws 30, 38 can be closed, and the shuttle 14 can be passed from the lower jaw 38 to the upper jaw 30, for example, by moving (e.g., pushing) the shuttle 14 from the shuttle second position in the lower jaw 38 to a position in the upper jaw 30.

For example, when the device 188 is in the device loaded configuration with the shuttle in the shuttle third position in the lower jaw 38, the device 188 can detached from the loader 750, the upper and lower jaws 30, 38 can be closed, and the shuttle 14 can be passed from the lower jaw 38 to the upper jaw 38, for example, by moving (e.g., pushing) the shuttle 14 from the shuttle third position in the lower jaw 38 to a position in the upper jaw 30.

As another example, the device 188 can be removed from the loader 750 once the shuttle 14 is unloaded from the device 188 (e.g., from the shuttle second or third position) to the shuttle first position onto the loader 750 (e.g., into the track 756).

Moving the loader control 762 from the loader control first position to the loader control second position can move the shuttle 14 from the shuttle first position to the shuttle second position or can cause the shuttle 14 to move from the shuttle first position to the shuttle second position. For example, when the loader control 762 is moved from the loader control first position to the loader control second position, the loader control 762 can pull the suture 70 which can in turn pull the shuttle 14. As the loader control 762 is moved in the first direction 765, for example, from the loader control first position to the loader control second position, the suture 70 between the shuttle 14 and the loader control 762 can be in tension. In this way the loader control 762 can move the shuttle 14 by moving the suture 70. As another example, the loader control 762 can be directly attached (e.g., directly removably attached) to the shuttle 14. As yet another example, the loader control 762 can be the suture 70 itself. For example, the user can pull on the suture 70 to load the shuttle 14 into the device 188.

As the loader control 762 is moved from the loader control first position to the loader control second position, the shuttle 14 can be moved along the loader body shuttle track 756, along the cap shuttle track 760, along the first jaw space 752a, along a jaw track (e.g., the upper jaw track 64 or the lower jaw track 66), or any combination thereof. For example, when the loader 750 is used to load the upper jaw 30 with the shuttle 14, the shuttle 14 can be moved along the loader body shuttle track 756 and the cap shuttle track 760 into the upper jaw track 64. When the shuttle 14 is in the upper jaw track 64, the shuttle can be in or above the first jaw space 752a. When the shuttle 14 is in the shuttle second position in the upper jaw 30, the loader control 762 can be in the loader control second position and the shuttle 14 can be fully loaded into the upper jaw track 64 (e.g., the shuttle 14 can be in a fully loaded position in the upper jaw track 64). As another example, when the loader 750 is used to load the lower jaw 38 with the shuttle 14, the shuttle 14 can be moved along the loader body shuttle track 756 and the cap shuttle track 760 into the lower jaw track 66. When the shuttle 14 is in the lower jaw track 66, the shuttle can be in or above the first jaw space 752a. When the shuttle 14 is in the shuttle second position in the lower jaw 38, the loader control 762 can be in the loader control second position and the shuttle 14 can be fully loaded into the lower jaw track 66 (e.g., the shuttle 14 can be in a fully loaded position in the upper jaw track 64).

When the loader control 762 is in the loader control second position, the shuttle 14 can be fully loaded into the device 188. Once the loader control 762 is in the loader control second position, the loader control 762 can be moveable to the loader control third position, for example, to help make sure the shuttle 14 is fully loaded into the device 188 by the loader control 762 and/or to let the user know that the shuttle 14 is fully loaded into the device 188. As the loader control 762 is moved from the loader control second position to the loader control third position, the shuttle 14 may or may not move farther into the device 188 (e.g., into the upper jaw 30 or into the lower jaw 38), for example, into the shuttle third position. In both cases, when the loader control 762 is in the loader control second position and the shuttle 14 is in the shuttle second position fully loaded into the device 188, moving the loader control 762 to the loader control third position can cause the loader 750 to provide tactile and/or audible feedback to the user (e.g., audible clicks, movement of the suture 70 through or around the loader control 762, an audible slipping sound as the suture 70 is pulled through or around the loader control 762) which can indicate that the shuttle 14 is fully loaded into the device 188. The loader control 762 can be moved from the loader control second position to the loader control third position to overload the shuttle 14 into the device. In such cases, as the loader control 762 is moved from the loader control second position to the loader control third position, the shuttle 14 can move farther into the device 188 (e.g., into the upper jaw 30 or into the lower jaw 38) to overload the shuttle 14 into the device 188. This can advantageously prevent or inhibit the male stop 412 of the shuttle 14 from getting locked or jammed with the female stop 416 in the jaw by creating a gap between the distal edge of the female stop 416 (e.g., the edge of the female stop 416 closest to the tip of the jaw) and the distal edge of the male stop 412 of the shuttle 14 that is in the female stop 416.

The loader control 762 can be moveable along the track 764 from the loader control first position to the loader control second position in discrete steps (e.g., stopping at one or more intermediate positions between the loader control first and second positions) and/or in one continuous movement, for example, in direction 765 (e.g., the direction toward the second terminal end of the track 764 along the track 764, for example, the direction along the track 764 from the first terminal end of the track 764 to the second terminal end of the track 764). The loader control 762 can be moved along the track 764 from the loader control first position to the loader control second position in discrete steps (e.g., stopping at one or more intermediate positions between the loader control first and second positions) and/or in one continuous movement, for example, in direction 765 (e.g., the direction toward the second terminal end of the track 764 along the track 764, for example, the direction along the track 764 from the first terminal end of the track 764 to the second terminal end of the track 764).

The loader control 762 can be moveable along the track 764 from the loader control first position to the loader control third position in discrete steps (e.g., stopping at one or more intermediate positions between the loader control first and second positions and/or stopping at one or more intermediate positions between the loader control second and third positions) and/or in one continuous movement, for example, in direction 765 (the direction along the track from the first terminal end of the track 764 to the second terminal end of the track 764). The loader control 762 can be moved along the track 764 from the loader control first position to the loader control third position in discrete steps (e.g., stopping at one or more intermediate positions between the loader control first and second positions and/or stopping at one or more intermediate positions between the loader control second and third positions) and/or in one continuous movement, for example, in direction 765 (the direction along the track from the first terminal end of the track 764 to the second terminal end of the track 764).

The loader control 762 can be moveable along the track 764 in a direction opposite to direction 765 (e.g., the direction toward the first terminal end of the track 764 along the track 764, for example, the direction along the track 764 from the second terminal end of the track 764 to the first terminal end of the track 764). The loader control 762 can be moved toward the first terminal end of the track 764, for example, to partially or fully unload the shuttle 14 from the device 188 back into the track 756. The shuttle 14 can be partially or fully unloaded from the upper jaw 30 or from the lower jaw 38, for example, if there was an error or malfunction during loading such as the shuttle 14 being misaligned with the upper or lower jaw 30, 38 or such as the suture 70 breaking or prematurely slipping through the loader control 762. As another example, the shuttle 14 can be partially or fully unloaded from the device 188 if when the loader control 762 is in the loader control second position or in the loader control third position, the shuttle 14 is in a partially loaded position between the shuttle first and second positions. Once the shuttle 14 is partially or fully unloaded from the device 188 by moving the loader control 762 in toward the first terminal end of the track 764 (e.g., in a direction opposite to direction 765), the shuttle 14 can be loaded or reloaded into the device with or without making adjustments to the loader 750 and/or the device 188. For example, after the shuttle 14 is unloaded from the device 188, the positions of the suture 70 and/or the shuttle 14 can be repositioned, as needed, so that the loader control 762 can load the shuttle 14 into the device 188. For example, if a portion of the suture 70 is extending across the track 756 after the shuttle is unloaded, the suture 70 can be moved away from the track 756 so that the suture 70 does not block or inhibit the shuttle 14 from moving along the track 756 and into the device 188 as the loader control 762 is moved from the loader control first position to the loader control second position.

FIG. 14A illustrates that the loader 750 can have a suture holder 766. The suture holder 766 can be, for example, a suture spool. The suture 70 can have a length of about 3 cm to about 150 cm, including every 1 cm increment within this range (e.g., 3 cm, 4 cm, 40 cm, 150 cm). The suture holder 766 may or may not hold suture, depending on the length of the suture. For example, where the suture holder 766 is a suture spool, the suture holder 766 may or may not have suture 70 wound around it. The loader 750 can be packaged with any length of suture. For example, FIG. 14A illustrates that the loader 750 can be packaged with a suture 70 having a length of 4 cm. For a suture length of 4 cm, the suture holder 766 may or may not hold any of the suture 70. As another example, FIG. 14A illustrates that the loader 750 can be packaged with a suture 70 having a length of 40 cm. For a suture length of 40 cm, the suture holder 766 can hold some of the suture 70 (e.g., where the suture holder 766 is a spool, some of the suture 70 can be wound around the suture holder 766). As the shuttle 14 is loaded into the device 188, for example, by moving the loader control 762 from the loader control first position to the loader control second position, the suture 70 on the suture holder 766 can be pulled from the suture holder 766. When the suture 70 is pulled from the suture holder 766, the suture 70 can, for example, unwind or unfold from the suture holder 766. As the suture 70 is pulled from the suture holder 766, the suture holder 766 can spin or rotate as the suture 70 is unwound from the suture holder 766. FIG. 14A illustrates, for example, that the suture holder 766 can be seen through holes (e.g., three holes) in the loader body 751.

The suture 70 can be a first suture. A first end of the suture 70 can be connected to the shuttle 14 (e.g., via the suture loop 162) and a second end of the suture 70 can be connected to a second suture, for example, to an implantable suture. The second end of the suture 70 can have a loop that can be attachable to the second suture. The first suture and/or the second suture can be removably attachable to the loader control 762.

FIG. 14A illustrates that the cap 758 can be on a first side of the loader 750 and that the suture holder 766 can be on a second side of the loader 750. The first and second sides of the loader 750 can be opposite to each other. For example, FIG. 14A illustrates that the cap can be on a front side of the loader 750 and that the suture holder 766 can be on the back side of the loader 750.

FIG. 14A illustrates that the suture 70 can extend from the shuttle 14 under the cap 758 to the loader control 762, can extend through the loader control 762 from a first side of the loader 750 to a second side of the loader 750, and can extend from the loader control 762 to the suture holder 766. As another example, FIG. 14A illustrates that the suture 70 can extend from the shuttle 14 under the cap 758 to the loader control 762, can extend through the loader control 762, can extend around the loader control 762, can extend from the first side of the loader 750 to the second side of the loader 750 (e.g., around the outside of the loader 750 or through the track 764), can extend to the suture holder 766, or any combination thereof.

FIG. 14A illustrates that the device 188 can be placed in the device space 752 with the upper jaw 30 in the first jaw space 752a with the upper and lower jaws 30, 38 in an open configuration (e.g., in a partially open configuration or in a fully open configuration). For example, FIG. 14A illustrates that the upper and lower jaws 30, 38 can be positionable in the first and second jaw spaces 752a, 752b, respectively (or vice versa), when the upper and lower jaws 30, 38 are in a fully open configuration.

When the upper and lower jaws 30, 38 are positioned in the first and second jaw spaces 752a, 752b, respectively (or vice versa), the shuttle 14 can be loaded into the device 188 with or without closing or clamping the upper and lower jaws 30, 38 against the cap 758. For example, when the upper and lower jaws 30, 38 are positioned in the first and second jaw spaces 752a, 752b, respectively (or vice versa), the upper and lower jaws 30, 38 can be closeable against the cap 758. For example, when the upper and lower jaws 30, 38 are positioned in the first and second jaw spaces 752a, 752b, respectively (or vice versa) in an open configuration, the upper and lower jaws 30, 38 can be partially closed against the cap 758, for example, by moving the jaw control 8 in direction 484 (e.g., by pulling the jaw control 8). When the upper and lower jaws 30, 38 are partially closed against the cap 758, the upper and lower jaws 30, 38 can be clamped against the cap 758. Closing the jaws against the cap 758 can advantageously stabilize the device 188 in the device space 752. Closing the jaws against the cap 758 can advantageously align the shuttle 14 with the jaw track that is in the first jaw space 752a (e.g., the upper jaw track 64 or the lower jaw track 66). As another example, when the upper and lower jaws 30, 38 are positioned in the first and second jaw spaces 752a, 752b, respectively (or vice versa), the upper and lower jaws 30, 38 can be clampable against the cap 758. For example, when the upper and lower jaws 30, 38 are positioned in the first and second jaw spaces 752a, 752b, respectively (or vice versa) in an open configuration, the upper and lower jaws 30, 38 can be clamped against the cap 758 by moving the jaws into a partially closed configuration (e.g., by moving the jaw control 8 in direction 484). Clamping the jaws against the cap 758 can advantageously stabilize the device in the device space 752. Clamping the jaws against the cap 758 can advantageously align the shuttle 14 with the jaw track that is in the first jaw space 752a (e.g., the upper jaw track 64 or the lower jaw track 66). As another example, when the upper and lower jaws 30, 38 are positioned in the first and second jaw spaces 752a, 752b, respectively (or vice versa), the upper and lower jaws 30, 38 can be closeable or clampable against the cap 758 but closing or clamping the upper and lower jaws 30, 38 against the cap 758 may not be necessary to stabilize the device 188 or to align the shuttle 14 with the jaw track that is in the first jaw space 752a before loading the shuttle 14 into the device 188, as the fit of the device 188 in the device space 752 can stabilize the device 188 and can align the shuttle 14 with the jaw track that is in the first jaw space 752a. As yet another example, when the upper and lower jaws 30, 38 are positioned in the first and second jaw spaces 752a, 752b, respectively (or vice versa), the fit of the device 188 in the device space 752 can be such that the upper jaw 30 and/or lower jaw 38 cannot be closed or clamped against the cap 758 as there may not be space for the upper and lower jaws 30, 38 to move in the first and second jaw spaces 752a, 752b.

FIG. 14A illustrates that when the shuttle 14 is in the shuttle first position (e.g., in the position shown in FIG. 14A), a portion 768 (also referred to as the exposed portion 768) of one of the shuttle tips 164 can extend from the cap 758. The portion 768 can be the portion of the shuttle 14 that extends out from under the cap 758 when the shuttle 14 is in the shuttle first position. The portion 768 can remain exposed outside of the cap 758 when the shuttle is in the shuttle first position. The exposed portion 768 can have an exposed length 768_{L}, for example, from about 0.15 mm to about 5.00 mm or more, including every 0.25 mm increment within this range (e.g., 0.15 mm, 1.00 mm, 1.50 mm, 5.00 mm). As another example, the exposed length 768_{L} can be the same as the exposed length 424.

When the jaws are in a partially closed configuration (e.g., with or without being clamped against the cap 758), all or a portion of the exposed portion 768 may not be in the jaw that is in the first jaw space 752a (e.g., the upper jaw 30 or the lower jaw 38). As another example, as the upper and lower jaws 30, 38 are partially closed against the cap 758, the jaw in the first jaw space 752a (e.g., the upper jaw 30 or the lower jaw 38) can close onto all or a portion of the exposed portion 768. The exposed portion 768 can align the jaw (e.g., the upper jaw 30 or the lower jaw 38) in the first jaw space 752a during loading, for example, as the jaw in the first jaw space 752a closes onto the exposed portion 768. For example, the device 188 can be placed in the device space 752 with the upper jaw 30 in the first jaw space 752a with the upper and lower jaws 30, 38 in an open configuration (e.g., in a partially open configuration or in a fully open configuration). When the jaws are in a partially closed configuration against the cap 758, all or a portion of the exposed portion 768 can be in the jaw that is in the first jaw space 752a (e.g., the upper jaw 30 or the lower jaw 38). When the shuttle 14 is in the shuttle first position and the jaws are closed against the cap 758, all or a portion of the exposed portion 768 can be in the jaw that is in the first jaw space 752a (e.g., the upper jaw 30 or the lower jaw 38). The exposed portion 768 can align the jaw that is in the first jaw space 752a as the upper and lower jaws 30, 38 are closed together against the cap 758. This can advantageously leverage the clamping force of the jaws to load the shuttle 14 into the device 188 (e.g., into the upper jaw 30 or into the lower jaw 38). When the shuttle 14 is in the shuttle second position, the portion 423 of the shuttle tip 164 can remain exposed outside of the jaw that is in the first jaw space 752a. In this way, a first side of the shuttle 14 can have the exposed portion 768 when the shuttle 14 is in the shuttle first position, and a second side of the shuttle 14 can have the exposed portion 423 when the shuttle 14 is in the shuttle second position. As another example, when the shuttle 14 is in the shuttle first position and the jaws are closed against the cap 758 and/or against the loader body 751, all or a portion of the exposed portion 768 can be outside of the jaw that is in the first jaw space 752a (e.g., the upper jaw 30 or the lower jaw 38). For example, when the shuttle 14 is in the shuttle first position and the jaws are closed against the cap 758 and/or against the loader body 751, all or a portion of the exposed portion 768 can be in the track 756 and/or in the track 760. As another example, when the shuttle 14 is in the shuttle first position and the jaws are closed against the cap 758 and/or the loader body 751, a first portion of the exposed portion 768 can be in the track 756 and/or in the track 760, and a second portion of the exposed portion 768 can be in (e.g., can extend into) the first jaw space 752 with or without also extending into the device 188. As the jaws are closed against the cap 758 and/or against the loader body 751, the upper jaw 30 or the lower jaw 38 may or may not close onto the shuttle 14 (e.g., onto the exposed portion 768). In either case (e.g., the shuttle 14 is in the jaws when the jaws are clamped against cap 758 and/or the loader body 751, or the shuttle 14 is not in the jaws when the jaws are clamped against the cap 758 and/or the loader body 751), closing the jaws onto the cap 758 and/or onto the loader 750 can advantageously leverage the clamping force of the jaws to load the shuttle 14 into the device 188 (e.g., into the upper jaw 30 or into the lower jaw 38), for example, by stabilizing the device 188 in or on the loader 750. The shuttle 14 can be loaded into the device from the track 756 and/or from the track 760 with or without clamping the jaws onto the cap 758 and/or onto the loader body 751.

The shuttle 14 can be loaded into the device 188 with or without closing the jaws against the cap 758.

The first jaw space 752a and/or the second jaw space 752b can be sized and shaped to form an interference fit (also referred to as a friction fit) with the upper jaw 30 and/or the lower jaw 38 when the upper and lower jaws 30, 38 are in the device spaces 752a, 752b, respectively (or vice versa). When the first jaw space 752a is sized and shaped to form an interference fit with the jaw positioned in the first jaw space 752a, the first jaw space 752a can advantageously stabilize the device 188 on the loader 750 by stabilizing the jaw in the first jaw space 752a and can advantageously align the shuttle 14 with the jaw track that is in the first jaw space 752a (e.g., the upper jaw track 64 or the lower jaw track 66).

FIG. 14A illustrates that the loader 750 can have a luer cap holder 770 that a luer cap (e.g., shown in FIG. 14B) can be removably secured to. The luer cap holder 770 can be a recess or a hole in the loader body 751. For example, FIG. 14A illustrates that the luer cap holder 770 can be a hole in the loader body 751.

FIG. 14A illustrates that the loader 750 can have a loader length 750_{L}. The loader length 750_{L} can be, for example, about 2.50 in. to about 5.00 in., including every 0.01 in. increment within this range (e.g., 2.50 in., 3.00 in., 3.50 in., 5.00 in.).

FIG. 14B illustrates that the device 188 can be placed in the device space 752. The device 188 can be removably attached to the loader 750 in the arrangement shown, with the upper jaw 30 in the first jaw space 752a and the lower jaw 38 in the second jaw space 752b, or vice versa. For example, FIG. 14B illustrates that the loader 750 can be used to load the upper jaw 30 with the shuttle 14. When the loader control 762 is moved from the loader control first position to the loader control second position, the shuttle 14 can be moved into the upper jaw 30, for example, from outside the upper jaw 30 in the shuttle first position to inside the upper jaw 30 in the shuttle second position.

FIG. 14B illustrates the device 188 attached to the loader 750 before the jaws are closed against the cap 758. As another example, FIG. 14B illustrates the shuttle 14 can be loaded into the device 188 without closing the jaws against the cap 758. Before the jaws are closed against the cap 758, the jaws can float in the first and second jaw spaces 752a, 752b and may or may not make contact with the cap 758. For example, FIG. 14B illustrates the upper and lower jaws 30, 38 floating in the first and second jaw spaces 752a, 752b, respectively (or vice versa), with the upper jaw 30 in contact with the cap 758 and with the lower jaw 38 not in contact with the cap 758. When the jaws are closed against the cap 758, the upper and lower jaws 30, 38 can be in contact with the cap 758. When the jaws are closed against the cap 758, the upper and lower jaws 30, 38 can be clamped against the cap 758. When the jaws are closed against the cap 758, the jaws may not float in the first and second jaw spaces 752a, 752b. The shuttle 14 can be loadable into the device 188 before or after clamping the jaws against the cap 758 such that the shuttle 14 can be loaded into the device 188 with or without the jaws floating in the first and second jaw spaces 752a, 752b.

FIG. 14B illustrates that when the jaws are in the first and second jaw spaces 752a, 752b, the exposed portion 768 may not be in the jaw that is in the first jaw space 752a.

FIG. 14B illustrates that the luer cap 772 can be removably attached to the luer cap holder 770.

FIG. 14B illustrates that the device 188 can be removably attached to the loader 750 via the holders 754. For example, FIG. 14B illustrates that the holders 754 can be removably attached the compression cover 34 of the device 188. As another example, FIG. 14B illustrates that the compression cover 34 of the device 188 can be removably attached to the holders 754. FIG. 14B illustrates that the holders 754 can be clips and that the device 188 can be snapped into the clips to removably secure the device 188 to the loader 750 during loading and/or unloading.

FIG. 14B illustrates that the cap 758 can be opaque.

FIG. 14C illustrates that the cap 758 illustrated in FIG. 14B can be transparent, showing the shuttle 14, the suture loop 162, and the suture 70 under the cap 758.

FIG. 14D illustrates that the loader 750 can have loading instructions 774. The loading instructions 774 can include, for example, a loading instruction first step 774a, a loading instruction second step 774b, and a loading instruction third step 774c. Each loading instruction step (e.g., loading instruction first, second, and third steps 774a, 774b, 774c) can include one or multiple instructions.

FIG. 14D illustrates that when the upper jaw 30 is to be loaded with the shuttle 14, the loading instruction first step 774a can include, for example, opening the jaws, moving the upper jaw pusher 86 to a fully retracted position, and placing the upper jaw 30 is in the first jaw space 752a and the lower jaw 38 is in the second jaw space 752b, or any combination thereof. For example, when the upper jaw 30 is to be loaded with the shuttle 14, the loading instruction first step 774a can include an instruction to load the device 188 onto the loader 750 with the trigger arrangement shown (e.g., with the jaw control 8 and the shuttle control 479 in the positions shown). As another example, the loading instruction first step 774a can include an instruction to move the jaw control 8 in direction 485 (e.g., which can be opposite to direction 484) to move the upper and lower jaws 30, 38 into an open configuration (e.g., into a fully open configuration), can include an instruction to move the shuttle control 479 to the fully advanced position in direction 488 to position the lower jaw pusher 76 in a fully advanced position and the upper jaw pusher 86 in a fully retracted position, or can include both instructions.

As another example, when the lower jaw 38 is to be loaded with the shuttle 14, the loading instruction first step 774a can include, for example, opening the jaws, moving the lower jaw pusher 76 to a fully retracted position, and placing the lower jaw 38 is in the first jaw space 752a and the upper jaw 30 is in the second jaw space 752b, or any combination thereof. For example, when the lower jaw 38 is to be loaded with the shuttle 14, the loading instruction first step 774a can include an instruction to load the device 188 onto the loader 750 with the jaw control 8 in the position shown in FIG. 14D and with the shuttle control 479 in the position shown, for example, in FIG. 13D). For example, the loading instruction first step 774a can include an instruction to move the jaw control 8 in direction 485 (e.g., which can be opposite to direction 484) to move the upper and lower jaws 30, 38 into an open configuration (e.g., into a fully open configuration), can include an instruction to move the shuttle control 479 to the fully advanced position in direction 486 (e.g., see FIG. 13D) to position the upper jaw pusher 86 in a fully advanced position and the lower jaw pusher 86 in a fully retracted position, or can include both instructions.

FIG. 14D illustrates that the loading instruction second step 774b can include moving the loader control 762 from the loader control first position to the loader control second position to move the shuttle 14 from the shuttle first position to the shuttle second position.

FIG. 14D illustrates that the loading instruction third step 774c can include removing the device 188 from the loader 750.

As another example, the loading instructions 774 can include a loading instruction step between the loading instruction first and second steps 774a, 774b that includes an instruction to close or clamp the jaws against the cap 758 by moving the jaw control in direction 484.

FIG. 14D illustrates that the loader control track 764 can have the arrangement shown. The loader control 762 is shown transparent for illustrative purposes only, for example, to show the portion of the loader control track 764 under the loader control 762. As another example, the loader control 762 can be transparent. A transparent loader control 762 can advantageously allow the user to observe the suture 70 in the loader control 762, for example, so that the user can see whether or not the loader control 762 is properly pulling the suture 70 during the loading process. A transparent loader control 762 can advantageously allow the user to inspect the loader control 762 to verify that the suture 70 is properly positioned in the loader control 762 prior to loading the shuttle 14 into the device 188.

FIG. 14D illustrates that the loader 750 can be packaged without the suture 70 or the suture loop 162 attached to the shuttle 14. FIG. 14D illustrates that the suture 70 or the suture loop 162 can be attached to the shuttle 14 in the arrangement shown, and that the suture 70 can be attached to or passed through the loader control 762 in the arrangement shown.

FIG. 14D illustrates that the shuttle 14 can be fully under the cap 758, for example, such that the shuttle 14 does not have the portion 768 extending out from under the cap 758 when the shuttle is in the shuttle first position (e.g., the position of the shuttle 14 in FIG. 14D).

FIG. 14D illustrates that the luer cap 772 can be covered by the loading instructions 774. The loading instructions 774 can indicate the location of the luer cap 772. For example, FIG. 14D illustrates that the luer cap 772 is under “LUER CAP” on the loading instructions 774. To remove the luer cap 772 from the loader 750, the loading instructions 774 (e.g., the portion of the loading instructions 774 having the loading instruction second and third steps 774b, 774c) can be removed from the loader 750 (e.g., like a pull tab). As another example, to remove the luer cap 772 from the loader 750, the luer cap 772 can be pushed through the portion of the loading instructions 774 that cover the luer cap 772. Pushing the luer cap 772 through the loading instructions 774 can tear the loading instructions 774. As yet another example, to remove the luer cap 772 from the loader 750, the luer cap 772 can be removed from the back side of the loader 750 without having to remove or tear through the loading instructions 774.

FIG. 14D illustrates that the loader control 762 can have a loader control first side 762_{FS} and a loader control second side 762_{SS}. The loader control first side 762_{FS} can be on a first side of the loader 750 and the loader control second side 762_{SS} can be on a second side of the loader 750. For example, FIG. 14D illustrates that the loader control first side 762_{FS} can be on a front side of the loader 750 and that the loader control second side 762_{SS} can be on the back side of the loader 750. As shown in FIG. 14D, the loader control 762 can extend through the track 764.

FIGS. 14C and 14D illustrate that the loader control 762 can releasably hold the suture 70. The loader control 762 can have or can be, for example, a suture holder 795. The suture holder 795 can be a loader control suture holder. The suture holder 795 can have the loader control first side 762_{FS} and the loader control second side 762_{SS}. For example, the loader control first side 762_{FS} can be moveable (e.g., squeezable) toward and away from the loader control second side 762_{SS} to releasably hold and release the suture 70, respectively. A portion of the loader control first side 762_{FS} can be moveable toward and away from the loader control second side 762_{SS}, for example, to releasably hold and release the suture 70, respectively. A portion of the loader control second side 762_{SS} can be moveable toward and away from the loader control first side 762_{FS}, for example, to releasably hold and release the suture 70, respectively.

The suture holder 795 can releasably hold or lock the suture 70 in the loader control 762 as shuttle 14 is loaded and/or unloaded from the device 188. For example, the suture holder 795 can releasably hold or releasably lock the suture 70 in the loader control 762 as the loader control 762 is moved from the loader control first position to the loader control second or third position. The loader control first side 762_{FS} can be moved into (e.g., pushed into, pressed against) the loader control second side 762_{SS} to releasably hold the suture 70 in the loader control 762. The loader control first side 762_{FS} can be moved away from or depressed from the loader control second side 762_{SS} to release the suture 70 from the loader control 762. For example, when the loader control first side 762_{FS} and the loader control second side 762_{SS} are pressed together, the suture 70 can be releasably held by the loader control 762 as the loader control 762 is moved in direction 765 (e.g., from the loader control first position to the loader control second or third position) or in the direction opposite to direction 765 (e.g., from the loader control second or third position to the loader control first position). When the suture 70 is releasably held by the loader control 762, the shuttle 14 can be loaded into the device 188 via the loader control 762 pulling the shuttle 14 into the device 188 by pulling on the suture 70 as the loader control 762 is moved in direction 765. When the suture 70 is releasably held by the loader control 762, the shuttle 14 can be unloaded from the device 188 via the loader control 762 pulling the shuttle 14 out of the device 188 by pulling on the suture 70 as the loader control 762 is moved in the direction opposite to direction 765.

The suture holder 795 can advantageously inhibit or prevent the suture 70 from slipping through the loader control 762 as the loader control 762 is moved from the loader control first position to the loader control second position. For example, the suture holder 795 can prevent the suture 70 from sliding through the suture holder 795 in the loader control 762 until a loader control threshold force is reached or exceeded. When the loader control threshold force is reached or exceeded, the suture 70 can slip or slide through the suture holder 795 in the loader control 762. In this way the suture holder 795 can advantageously inhibit or prevent the suture 70 from breaking or fraying, allowing the suture to slip or slide through the loader control 762 when the loader control 762 pulls the suture 70 and the shuttle 14 with a force equal to or greater than the loader control threshold force. The loader control threshold force can advantageously inhibit or prevent the integrity of the suture 70 from breaking or becoming damaged during the loading process. The suture holder 795 can advantageously allow the loader control 762 to pull on the suture 70 to load the shuttle 14 into the device 188, to unload the shuttle 14 from the device 188, or both. The loader control threshold force can be, for example, from about 2.00 lbs to about 10.00 lbs, including every 0.01 lb increment within this range (e.g., 2.00 lbs, 3.00 lbs, 6.00 lbs, 10.00 lbs). The loader control threshold force can be higher than the loading force needed for loading the shuttle 14 into the device 188 or the unloading force need for unloading the shuttle 14 from the device 188. For example, the loading force can be about 0.30 lbs to about 4.00 lbs, including every 0.01 lb increment within this range (e.g., 0.30 lbs, 0.50 lbs, 2.00 lbs, 4.00 lbs). For example, the unloading force can be about 0.30 lbs to about 4.00 lbs, including every 0.01 lb increment within this range (e.g., 0.30 lbs, 0.50 lbs, 2.00 lbs, 4.00 lbs).

FIG. 14D illustrates that the loader control 762 can have one or multiple loader control suture tracks 775 (also referred to as the tracks 775) that the suture 70 can be positionable in and/or that the suture 70 can be moveable in. The suture 70 can move (e.g., slide, translate) in the tracks 775, for example, as the suture 70 is pulled through the loader control 762. The tracks 775 can be one or multiple grooves or channels in the loader control 762. For example, FIG. 14D illustrates that the loader control first side 762_{FS} can have a track 775 and that the loader control second side 762_{SS} can have a track 775. The tracks 775 can advantageously keep the suture 70 at or below a surface of the loader control 762 so that the suture is not damaged by the user (e.g., via pulling, twisting, or rolling the suture 70) as user moves the loader control 762 from the loader control first position to the loader control second or third position.

FIG. 14E illustrates that the loader 750 can be packaged with the suture 70 attached to the shuttle 14, for example, via the suture loop 162. Half of the upper jaw 30 in FIG. 14E is shown transparent for illustrative purposes only, for example, so that the relationship between the upper and lower jaw tracks 64, 66 and the shuttle 14 when the shuttle 14 is in the shuttle first position can be more easily seen.

FIG. 14E illustrates that when the jaws are in the first and second jaw spaces 752a, 752b, the exposed portion 768 can be aligned with the shuttle track of the jaw that is in the first jaw space 752a. For example, FIG. 14E illustrates that when the upper jaw 30 is in the first jaw space 752a, the shuttle tip 164 can be aligned with the upper jaw track 64 such that the shuttle 14 can be loaded into the upper jaw 30 when the loader control 762 is moved from the loader control first position to the loader control second or third position. As another example, example, when the lower jaw 38 is in the first jaw space 752a, the shuttle tip 164 can be aligned with the lower jaw track 66 such that the shuttle 14 can be loaded into the lower jaw when the loader control 762 is moved from the loader control first position to the loader control second or third position.

FIG. 14E illustrates, for example, that the loader 750 can have a loader body suture track 776 (also referred to as the suture track 776 or the track 776). The track 776 can be a track in the loader body 751. The track 776 can be a groove in the loader body 751. The track 776 can be a channel in the loader body 751. The track 776 can be a ledge. For example, FIG. 14E illustrates that the track 776 can be a ledge. The track 776 can be parallel to the track 756. The track 776 can have a radius of curvature greater than, less than, or equal to the radius of curvature of the shuttle 14. The track 776 can have a radius of curvature greater than, less than, or equal to the radius of curvature of the track 756. The suture 70 and/or the suture loop 162 can be in the track 776 or can be positionable in the track 776. For example, FIG. 14E illustrates that the suture loop 162 can be in the track or can be positionable in the track 776. For example, FIG. 14E illustrates that the loader 750 can be packaged with the suture loop 162 attached to the shuttle 14 and positioned in the track 776 in the arrangement shown, for example, when the shuttle 14 is in the shuttle first position and the loader control 762 is in the loader control first position.

The suture 70 and/or the suture loop 162 can be moveable (e.g., translatable, slidable) in the track 776. The suture 70 and/or the suture loop 162 can be longitudinally moveable along the track 776. For example, the suture 70 and/or the suture loop 162 can be translatable or slidable along the track 776. The suture 70 and/or the suture loop 162 can be moveable out of (e.g., longitudinally out of) the track 776, for example, into the upper jaw track 64 and/or into the upper jaw suture slot 238b. The suture 70 and/or the suture loop 162 can be moveable out of (e.g., longitudinally out of) the track 776, for example, into the lower jaw track 66 and/or into the lower jaw suture slot 238a. For example, when the shuttle 14 is in the shuttle first position, FIG. 14E illustrates that the shuttle 14 can be in the track 756 and that the suture loop 162 can be in the track 776. When the shuttle 14 is in the shuttle second position and has been loaded into the upper jaw 30, the shuttle 14 can be in the upper jaw 30, and the suture loop 162 can be in the upper jaw track 64 and/or in the upper jaw suture slot 238b. When the shuttle 14 is in the shuttle second position and has been loaded into the lower jaw 38, the shuttle 14 can be in the lower jaw 38, and the suture loop 162 can be in the lower jaw track 66 and/or in the lower jaw suture slot 238a. The suture 70 and/or the suture loop 162 can be movable in the track 776 as the shuttle 14 is loaded into the device 188 in direction 763. The suture 70 and/or the suture loop 162 can be movable in the track 776 as the shuttle 14 is unloaded from the device 188 in a direction opposite to direction 763.

FIG. 14E illustrates that the loader body 751 can have a loader body surface 778 (also referred to as the surface 778). The surface 778 can form an edge of the track 776. The surface 778 can form an edge of the first jaw space 752a. The surface 778 can form an edge of the second jaw space 752b. For example, FIG. 14E illustrates that the surface 778 can abut or form the edge of the first and second jaw spaces 752a, 752b. FIG. 14E illustrates that the male stops 412 on the shuttle 14, the suture loop 162, and the suture 70 can extend over the surface 778. In this way, the face of the loader 750 (e.g., the surface 778) can be a track over which the male stops 412 on the shuttle 14, the suture loop 162, and the suture 70 can extend. As another example, lateral male stops (e.g., the lateral male stops 412 shown in FIGS. 11F and 11G) may not extend over the surface 778. The male stops 412 on the shuttle 14, the suture loop 162, and the suture 70 can be moveable across the surface 778, for example, as the shuttle 14 is moved from the shuttle first position to the shuttle second position. The male stops 412 on the shuttle 14, the suture loop 162, and/or the suture 70 may or may not contact the surface 778. For example, as the shuttle 14 is moved from the shuttle first position to the shuttle second position, the male stops 412 on the shuttle 14, the suture loop 162, and/or the suture 70 may or may not contact the surface 778. For example, FIG. 14E illustrates that as the shuttle 14 is moved from the shuttle first position to the shuttle second position, the male stops 412 on the shuttle 14, the suture loop 162, and/or the suture 70 can extend over the surface 778 without contacting the surface 778 as the male stops 412 on the shuttle 14, the suture loop 162, and/or the suture 70 are moved across the surface 778 by the loader control 762 as the loader control 762 is moved, for example, from the loader control first position to the loader control second or third position.

FIG. 14F illustrates the device 188 in the loader 750 with the cap 758 shown transparent for illustrative purposes only. As another example, FIG. 14F illustrates that the loader 750 may not have a cap (e.g., the cap 758).

FIG. 14F illustrates that the first jaw space 752a can be deeper (e.g., have a greater height) than the track 756, and that the track 756 can be deeper (e.g., have a greater height) than the track 776. For example, the first jaw space 752a can have a first jaw space depth, the track 756 can have a loader body shuttle track depth, and the track 776 can have a loader body suture track depth. FIG. 14F illustrates, for example, that the first jaw space depth can be greater than the loader body shuttle track depth, and the loader body shuttle track depth can be greater than the loader body suture track depth. FIG. 14F illustrates that the surface 778 and the bottom surface of the track 776 can be the same level as each other. As another example, the track 776 can be deeper than the surface 778. As yet another example, the surface 778 can form the track 776.

FIG. 14F illustrates that the suture 70 can extend from the shuttle 14 toward the loader control 762 and loop around the loader 750 from a first side of the loader 750 to a second side of the loader 750 through the loader control 762. For example, FIG. 14F illustrates that the suture 70 can extend through the loader control 762 (e.g., through the suture holder 795), can extend through the track 775 in the loader control first side 762_{FS}, and can extend through the track 775 in the loader control second side 762_{SS}.

FIG. 14F illustrates that the lower jaw 38 can be placed in the first jaw space 752a and that the upper jaw 30 can be placed in the second jaw space 752b.

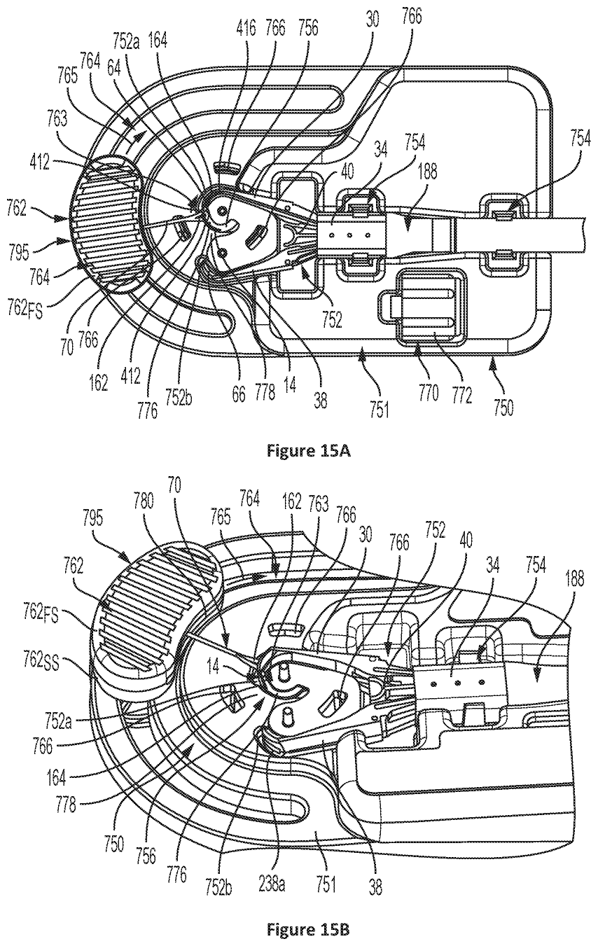

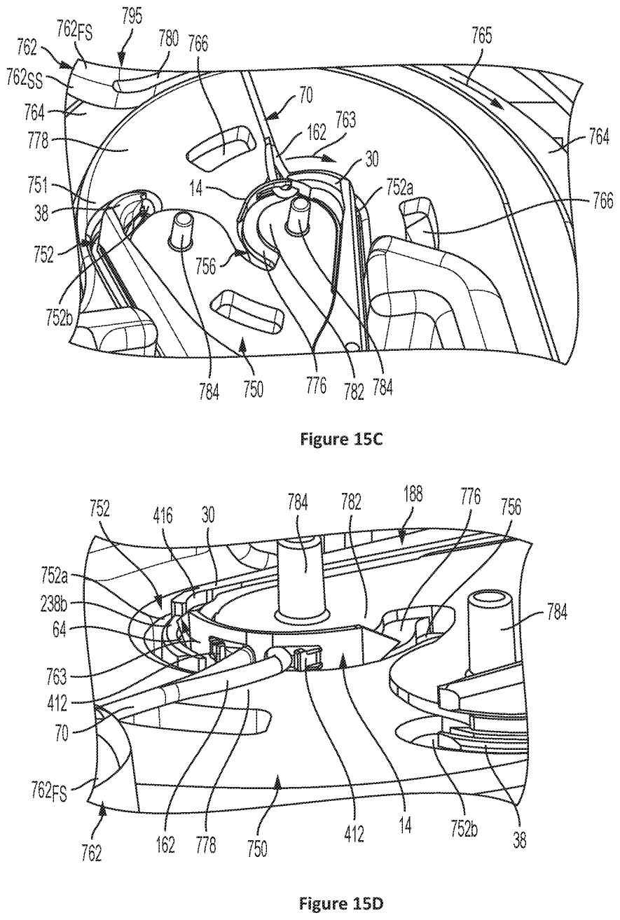

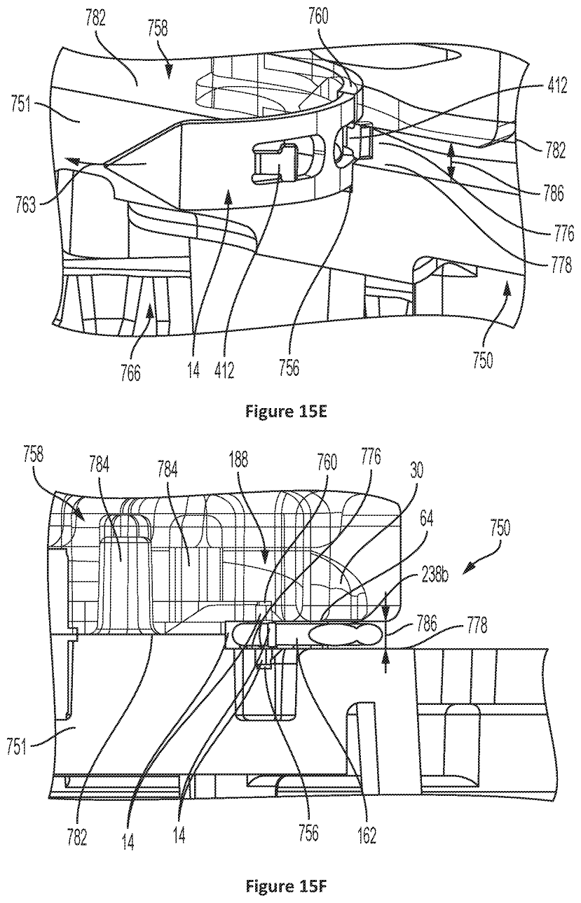

FIG. 15A illustrates that the jaws can be clamped against the loader body 751 (e.g., by pulling the jaw control 8). When the jaws are clamped against the loader body 571, the shuttle 14 can be loaded into the device 188 via the loader control 762. As another example, half of the jaws can be clamped against the loader body 751 and half of the jaws can be clamped against the cap 758 (e.g., shown transparent in FIG. 15A). FIG. 15A shows the loader 750 and the device 188 of FIG. 14E in a mid-loaded configuration (also referred to as a partially loaded configuration). As for FIG. 14E, half of the upper and lower jaws 30, 38 in FIG. 15A are shown transparent for illustrative purposes only, for example, so that the relationship between the upper and lower jaw tracks 64, 66 and the shuttle 14 when the shuttle 14 is between the shuttle first and second positions can be more easily seen.

FIG. 15A illustrates the loader control 762 can be moved away from the loader control first position to a loader control intermediate position between the loader control first and second positions. FIG. 15A illustrates that when the loader control 762 is in an intermediate position, the shuttle 14 can be between the shuttle first and second positions, for example, in a shuttle intermediate position. The intermediate position of the loader control illustrated in FIG. 15A is also referred to as the loader control first intermediate position. The intermediate position of the shuttle 14 illustrated in FIG. 15A is also referred to as the shuttle first intermediate position.

FIG. 15A illustrates, for example, the loader 750 in a partially loaded configuration and the device 188 in a partially loaded configuration. When the loader 750 is in a partially loaded configuration, the loader control 762 can be between the first and second terminal ends of the track 764. When the loader 750 is in a partially loaded configuration, the loader control 762 can be between the loader control first position and the loader control second position, for example, in an intermediate position between the loader control first and second positions as shown in FIG. 15A. When the loader 750 is in a partially loaded configuration, the suture loop 162 can extend over the track 756, over the track 776, and over the surface 778. When the device 188 is in a partially loaded configuration, a first end of the shuttle 14 can be in the device 188 (e.g., in the upper jaw 30 or in the lower jaw 38) and a second end of the shuttle 14 can be outside of the device 188, for example, in the track 756. When the loader 750 is in a partially loaded configuration, the shuttle 14 can be between the shuttle first position and the shuttle second position, for example, in an intermediate position between the shuttle first and second positions as shown in FIG. 15A. When the device 188 is in a partially loaded configuration, the suture loop 162 can extend over the track 756, over the track 776, and over the surface 778.

FIG. 15B illustrates that the loader control 762 can be moved away from the loader control first intermediate position to a loader control second intermediate position, where the loader control second intermediate position can be between the loader control first and second positions. The loader control second intermediate position can be between the loader control first intermediate position and the loader control second position. FIG. 15B illustrates that when the loader control 762 is in the loader control second intermediate position, the shuttle can be between the shuttle first and second positions, for example, in a shuttle second intermediate position. The shuttle second intermediate position can be between the shuttle first intermediate position and the shuttle second position.

FIG. 15B illustrates, for example, the loader 750 in a partially loaded configuration and the device 188 in a partially loaded configuration. When the loader and the device 750, are in a partially loaded configuration and the loader control 762 is in the loader control second intermediate position, the suture loop 162 can be in the upper jaw 30, extend through the upper jaw suture slot 238b, and extend over both the first jaw space 752a and the surface 778. FIG. 15B illustrates the suture 70 and the suture loop 162 can be fully out of the track 776 when the device 188 is in a partially loaded configuration. When the device 188 is in a partially loaded configuration and the loader control 762 is in the loader control second intermediate position, a first end of the shuttle 14 can be in the device 188 (e.g., in the upper jaw 30 or in the lower jaw 38) and a second end of the shuttle 14 can be outside of the device 188, for example, in the track 756. FIG. 15B illustrates that both of the male stops 412 illustrated in FIG. 15A can be in the upper jaw 30 (e.g., they no longer extend over the surface 778). In the partially loaded configuration shown in FIG. 15B, the male stops 412 may not be engaged with a female stop 416.

FIG. 15B illustrates that the loader control 762 (e.g., the loader control first side 762_{FS}) can have a loader control channel 780 (also referred to as the channel 780). The channel 780 can be the opening to the suture holder 795. FIG. 15B illustrates that the suture 70 can extend into the channel 780, for example, from the shuttle 14.

FIG. 15C illustrates that the surface 778 and the track 776 can have the same height, for example, as measured from the bottom of the track 756. As another example, the surface 778 can define the track 776.

FIG. 15C illustrates that the loader body 751 can have a loader body surface 782 (also referred to as the surface 782). FIG. 15C illustrates that the cap 758 can be placed on and/or over the surface 782. The surface 782 can be above the track 776 and the surface 778, for example, to provide clearance (e.g., a gap, a space) between the cap 758 and the surface 778 for the suture 70, the suture loop 162, and/or the male stops 412 to extend into when the cap 758 is attached to the loader 750. The cap 758 can be attached to (e.g., glued to) or rest against the surface 782. The cap 758 can be attached to attachers 784. The attachers 784 can be, for example, posts or masts. For example, the cap 758 can be glued to the attachers 784. As another example, the cap 758 can be removably attachable to the attachers 784, for example, with a friction fit, a snap fit, or a magnetic fit. When the cap 758 is attached to the attachers 784, the cap 758 can contact or rest against the surface 782.

FIG. 15D illustrates that when the loader control 762 is in a loader control intermediate position, for example, the intermediate position shown in FIG. 15C, the loader 750 and the device 188 can have the arrangement of features shown, for example, with the suture 70 extending across the surface 778 to the loader control 762.

FIG. 15E illustrates that when the cap 758 is attached to the loader 750, a bottom surface of the cap 758 can be in contact with the surface 782.

FIG. 15E illustrates that when the cap 758 is attached to the loader 750, a space 786 (also referred to as a gap 786) can be between the cap 758 and the surface 778 for the suture 70, the suture loop 162, and/or the male stops 412 to extend into and/or move through. The suture 70, the suture loop 162, and/or the male stops 412 can be moveable in the space 786. FIG. 15E illustrates that the surface 778 can form the track 776.

FIGS. 15E and 15F illustrate that the track 756 can be opposite of the track 760 such that a first lateral side of the shuttle 14 is moveable in the track 756 and a second lateral side of the shuttle 14 is moveable in the track 760.

FIG. 15F illustrates that the suture loop 162 can be in the gap 786. The suture loop 162 can be in the gap 786, for example, when the loader control 762 is in the loader control first position, is in the loader control second position, and/or is in any position between the loader control first and second positions. As another example, the suture loop 162 may no longer extend through the gap 786 when the loader control 762 is in the loader control second position. For example, FIG. 15F can illustrate a cross-sectional view of FIG. 14A with the cap 758 attached to the loader 750. As another example, FIG. 15F can illustrate a cross-sectional view of FIG. 15A with the cap 758 attached to the loader 750. As yet another example, FIG. 15F can illustrate a cross-sectional view of FIG. 15B with the cap 758 attached to the loader 750.

FIG. 15F illustrates that the gap 786 can be between the cap 758 and the loader body 751, for example, to inhibit or prevent the suture 70 from lifting out of the plane that extends, for example, between the shuttle 14 and the loader control 762, and that FIG. 15F shows can be parallel to the surface 778.

FIG. 15F illustrates that the surface defining the track 776 and the surface 778 can be same level as each other. For example, the surface defining the track 776 and the surface 778 can be the same distance away from the bottom of the cap 758. As another example, the surface defining the track 776 and the surface 778 can be the same distance (e.g., height) away from the bottom of the track 756.

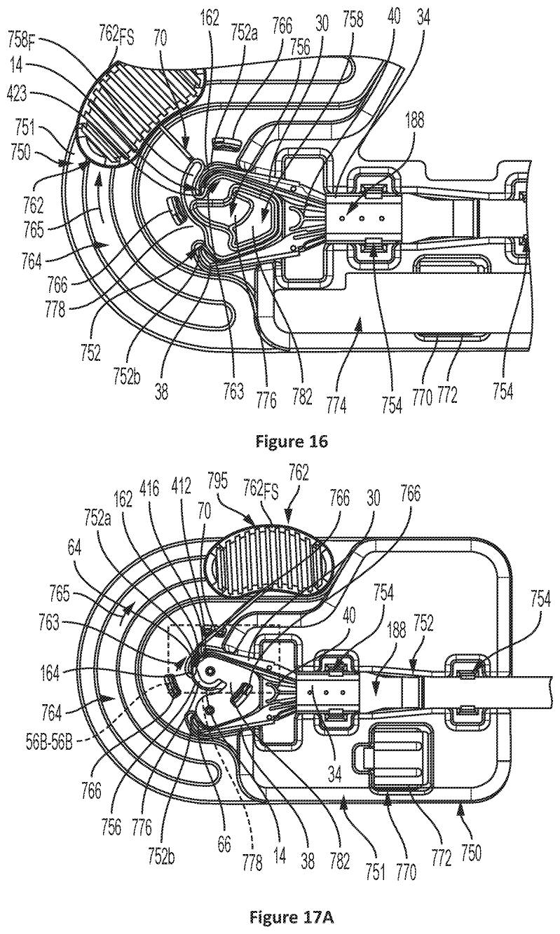

FIG. 16 illustrates that the loader control 762 can be moved to the loader control second position. For example, FIG. 16 illustrates that the loader control 762 can be moved away from the loader control second intermediate position to the loader control second position. FIG. 16 illustrates that when the loader control 762 is in the loader control second position, the loader 750 and the device 188 can have the arrangement of features as shown. FIG. 16 illustrates, for example, that when the loader control 762 is in the loader control second position, the shuttle 14 can be in the shuttle second position, and that the shuttle second position can be a fully loaded position. When the loader control 762 is in the loader control second position, FIG. 16 illustrates that a male stop (e.g., a male stop 412) can be engaged with a female stop. When the loader control 762 is moved into the loader control second position (e.g., from the loader control first position), the shuttle 14 can be moved out of the tracks in the shuttle body 751 and the cap 758 (e.g., tracks 756 and 760) into the device 188 (e.g., into the upper jaw 30 or into the lower jaw 38), the suture loop 162 can be moved out of or off of the track 776 onto or over the surface 778 and/or onto or over the first jaw space 752a, and the suture 70 can be moved out of or off of the track 776 and onto or over the surface 778 and/or onto or over the first jaw space 752a, or any combination thereof. FIG. 16 illustrates that when the loader control 762 is in the loader control second position, the exposed portion 423 can extend from the device 188. As another example, when the loader control 762 is in the loader control second position and the shuttle 14 is in the shuttle second position, the shuttle 14 can be fully in the device 188, for example, such that the shuttle 14 does not extend from the device 188 or does not have an exposed portion (e.g., exposed portion 423).

FIG. 16 illustrates that the suture 70 and/or the suture loop 162 can extend under the cap 758 (e.g., under the finger 758_{F}), for example, through the space 786, when the loader control 762 is in the loader control second position shown in FIG. 16.

FIG. 16 illustrates that the cap 558 can be placed over the track 756, over the track 776, over a portion of the surface 778, and over the surface 782, or over any combination thereof. The cap 558 in FIG. 16 is shown opaque, so the track 756, the track 760, the track 776, the portion of the surface 778 covered by the cap 758, and the surface 782 in FIG. 16 are shown under the cap 758.

FIG. 16 illustrates that half of the jaws can be clamped against the loader body 751 and that half of the jaws can be clamped against the cap 758. As another example, FIG. 16 illustrates that the jaws of the device 188 may not be clamped and that the first jaw space 752a can be smaller than the second jaw space 752b such that the jaw in the first jaw space 752a can have a tighter fit with the loader 750 than the other jaw in the second jaw space 752b. FIG. 16 illustrates for example, that the jaw placed in the first jaw space 752a may not float in the first jaw space 752a (e.g., may not have wiggle room in the first jaw space 752a) and that the jaw placed in the second jaw space 752b can float in second jaw space 752b (e.g., can have wiggle room in the second jaw space 752b). This can inhibit or prevent the fit between the device 188 and the loader 750 from being too tight while still stabilizing the jaw that is in the first jaw space 752a for loading and/or unloading of the shuttle 14 so that the user can easily attach and detach the device 188 to and from the device space 752. For example, FIG. 16 illustrates that the space between a wall of the loader body 751 (e.g., the portion of the wall below the reference numeral 756 shown contacting the upper jaw 30 in FIG. 16) and a wall of the cap 758 (e.g., the portion of the cap 758 opposite the portion of the wall below the reference numeral 756 shown contacting the upper jaw 30 in FIG. 16) can be narrower than the same space for the other jaw in the second jaw space 752b. This can result in a more secure fit for the jaw in the first jaw space 752a than the jaw in the second jaw space 752b. The fit can be, for example, an interference fit. As another example, the fit may not be an interference fit but the fit may not allow the jaw in the first jaw space 752a to float. As yet another example, the fit can allow the jaw in the first jaw space 752a to float, for example, by 0.50 mm to 1.00 mm, or more broadly from 0.50 mm to about 1.50 mm, including every 0.01 mm increment within these ranges (e.g., 0.50 mm, 1.00 mm, 1.50 mm). FIG. 16 illustrates that the tolerance in the second jaw space 752b can allow the jaw that is in the second jaw space to float such that there is 0.10 mm to about 2.00 mm of space between the jaw and the wall of the loader body 751 defining the second jaw space 752b, including every 0.01 mm increment within this range (e.g., 0.10 mm, 1.00 mm, 2.00 mm). Although the jaw that is in the second jaw space 752b can float in the second jaw space 752b, the jaw that is in the second jaw space (e.g., the lower jaw 38 in FIG. 16) may or may not be movable in the second jaw space 752b, for example, toward and/or away from the cap 758.

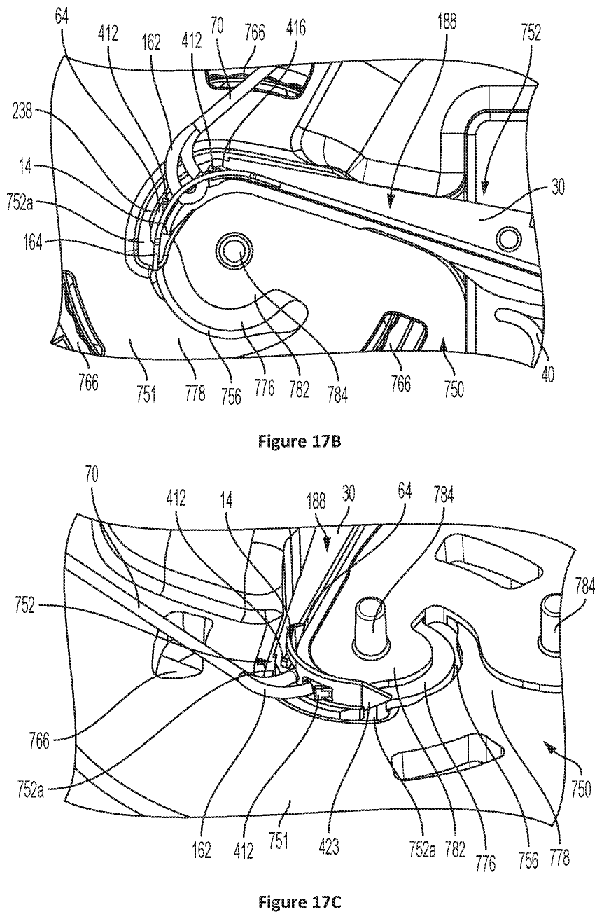

FIG. 17A illustrates that the loader control 762 can be moved to the loader control third position. FIG. 17A illustrates that when the loader control 762 is in the loader control third position, the loader 750 and the device 188 can have the arrangement of features as shown. FIG. 17A illustrates, for example, that when the loader control 762 is in the loader control third position, the shuttle 14 can be in the shuttle second position, and that the shuttle second position can be a fully loaded position. As another example, when the loader control 762 is in the loader control third position, the shuttle 14 can be in a shuttle third position, which can be an overloaded position of the shuttle 14 in the device 188. When the shuttle 14 is in the shuttle third position, more of the shuttle 14 can be in the device 188 than when the shuttle 14 is in the shuttle second position. For example, when the shuttle 14 is in the shuttle second position, the exposed portion 423 can extend from the device 188, and when the shuttle 14 is in the shuttle third position, less of the exposed portion 423 can extend from the device 188 or the shuttle 14 can be completely in the device 188. In such cases, moving the loader control 762 from the loader control second position to the loader control third position can move the shuttle 14 about 0.10 mm to about 3.00 mm further into the device 188, or more narrowly, about 0.10 mm to about 1.50 mm further into the device 188, including every 0.01 mm increment within these ranges (e.g., 0.10 mm, 0.50 mm, 1.50 mm, 3.00 mm). As another example, when the shuttle 14 is in the shuttle third position, the shuttle 14 can be farther in the device 188 than when the shuttle 14 is in the shuttle second position such that the shuttle 14 is completely in the device 188 when the shuttle 14 is in the shuttle second position and when the shuttle 14 is in the shuttle third position (e.g., there may not be an exposed portion 423 when the shuttle 14 is in the shuttle second position and there may not be an exposed 723 when the shuttle 14 is in the shuttle third position). In such cases, moving the loader control 762 from the loader control second position to the loader control third position can move the shuttle 14 about 0.10 mm to about 3.00 mm further into the device 188, or more narrowly, about 0.10 mm to about 1.50 mm further into the device 188, including every 0.01 mm increment within these ranges (e.g., 0.10 mm, 0.50 mm, 1.50 mm, 3.00 mm). As yet another example, the shuttle 14 can have the same position (e.g., the shuttle second position) when the loader control 762 is in the loader control second position and when the loader control 762 is in the loader control third position.

FIG. 17A illustrates that when the loader control 762 is in the loader control third position, the loader control 762 can be at the second terminal end of the track 764. When the loader control 762 is in the loader control third position, the suture 70 and the suture loop 162 can be in the positions shown. When the loader control 762 is in the loader control third position, FIG. 17A illustrates that a male stop can be engaged with a female stop. As the loader control 762 is moved from the loader control second position to the loader control third position, the suture 70 may not slip through the suture holder 795. As the loader control 762 is moved from the loader control second position to the loader control third position, the suture 70 can slip through the suture holder 795. The suture holder 795 can be a friction member that the suture 70 can slip through. As for FIGS. 14E and 15A, the cap 758 and half of the upper and lower jaws 30, 38 in FIG. 17A are shown transparent for illustrative purposes only, for example, so that the relationship between the upper and lower jaw tracks 64, 66 and the shuttle 14 when the shuttle 14 is in the shuttle third position can be more easily seen.

FIG. 17B illustrates that when the loader control 762 is moved into the loader control third position (e.g., from the loader control first position), the shuttle 14 can be moved out of the track 756 into the device 188 (e.g., into the upper jaw 30 or into the lower jaw 38), the suture loop 162 can be moved out of or off of the track 776 onto or over the surface 778 and/or onto or over the first jaw space 752a, the suture 70 can moved out of or off of the track 776 and onto or over the surface 778 and/or onto or over the first jaw space 752a, the suture 70 and/or the suture loop 162 can be moved into the suture holder slot 238 of the jaw that is in the first jaw space 752a, or any combination thereof.

FIG. 17B illustrates that when the loader control 762 is in the loader control third position, the exposed portion 423 can extend from the device 188. As another example, when the loader control 762 is in the loader control second position and the shuttle 14 is in the shuttle second position, the shuttle 14 can be fully in the device 188, for example, such that the shuttle 14 does not extend from the device 188, does not have an exposed portion (e.g., exposed portion 423), or less of the exposed portion 423 is exposed when the loader control 762 is in the loader control third position than when in the loader control second position.

FIG. 17C illustrates the arrangement of features shown, for example, that the surface can be a ledge, that the track 776 can be a ledge, that the track 756 can be a channel, that the surface 778 can form a ledge that abuts and forms a wall of the track 756, and that the surface 778 and the surface forming the track 776 can be the same height (e.g., the same height above the bottom of the track 756.

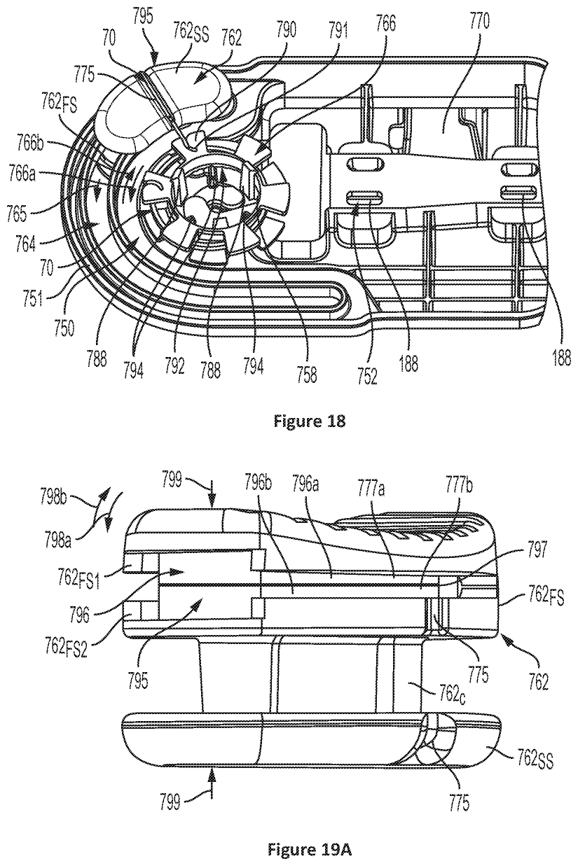

FIG. 18 illustrates that the suture holder 766 can be attached to (e.g., removably attached to) the loader 750. The suture holder 766 can be attached to the front side or the back side of the loader 750. For example, FIG. 18 illustrates that the suture holder 766 can be attached to the back side of the loader 750.

FIG. 18 illustrates that the loader 750 can have one or multiple holders 788 that can hold the suture holder 766 and the loader 750 together. For example, FIG. 18 illustrates that the holders 788 can hold the suture holder 766 on the back side of the loader 750. The holders can be, for example, clips, clasps, magnets, or fasteners, clasps, catches, pins, or any combination thereof. For example, FIG. 18 illustrates that the holders 788 can be clips that the suture holder 766 can be snapped into. FIG. 18 illustrates that the loader 750 can be packaged with the suture holder 766 attached to the loader 750, for example, in the arrangement shown with suture 70 on (e.g., wound around) the suture holder 766. The holders can be part of (e.g., integrated with) the loader body 751. The holders 788 can be extensions of the loader body 751. The holders 788 can extend from the loader body 751. FIG. 18 illustrates that the loader 750 can have three holders 788, and that the holders 788 can be evenly spaced apart, for example, every 60 degrees.

FIG. 18 illustrates that the suture holder 766 can be rotatable for example, in direction 766a, in direction 766b, or in directions 766a and 766b. When the suture holder 766 is attached to the loader 750 (e.g., in the position shown in FIG. 18), the suture holder 766 can be rotatable (e.g., in directions 766a and/or 766b). For example, the suture holder 766 can rotate in direction 766a as the loader control 762 is moved (e.g., from the loader control first position to any subsequent position, for example, to the loader control second position and/or to the loader control third position). As another example, the suture holder 766 can rotate in direction 766b as the loader control 762 is moved (e.g., from the loader control second or third position to any subsequent position, for example, to the loader control first position). The suture 70 can release (e.g., passively release) from the suture holder 766 as the suture holder 766 rotates in direction 766a. As another example, the suture holder 766 can rotate in direction 766a as the loader control 762 is moved but the suture 70 may not begin releasing (e.g., passively releasing) from the suture holder 766 until the loader control 762 is in a suture release position at which the suture 70 can begin to unwind from the suture holder 766. The loader control suture release position can be, for example, an intermediate position between the loader control first and second positions or the loader control second position or beyond such that the suture 70 may not begin to unwind from the suture holder 766 as the loader control 762 is moved in direction 765 until the loader control 762 is in the loader control suture release position. For example, FIG. 18 illustrates that the suture holder 766 can have a suture grabber 790 that can inhibit or prevent the suture 70 from releasing (e.g., unspooling, unwinding) from the suture holder 766 until the loader control 762 is in the loader control suture release position (e.g., the loader control second position or beyond). This can advantageously keep the suture 70 in tension while the shuttle 14 is being loaded into the device 188 via the loader control 762. For example, the suture grabber 790 can have a track 791 that the suture 70 can extend through. The track 791 can be a channel, a slot, a groove, or a hole in the suture holder 766. For example, FIG. 18 illustrates that the track 791 can be a slot, a groove, or a channel. The track 791 can have curve (e.g., the crescent curve shown) so that the suture 70 is inhibited from releasing from the suture grabber 790 until the loader control 762 is in the suture release position (e.g., the loader control second position) and/or until the loader control threshold force is reached or exceeded. Once the loader control 762 is in the loader control suture release position (e.g., the loader control second position), further movement of the loader control 762 in direction 765, for example, to the loader control third position, can release the suture 70 from the suture grabber 790 by turning the suture holder 766 to such an extent that that the suture 70 can be pulled out of the track 791 as the loader control 762 is moved beyond the loader control second position. Once the suture 70 is pulled out of the track 791, the suture 70 can release (e.g., unwind, unspool) from the suture holder 766.

When the loader control 762 is in any position, the user can pull the suture 70 (e.g., with their hands) to release (e.g., actively release) the suture 70 from the suture holder 766. Passive release from the suture holder 766 can be when movement of the loader control 762 releases the suture 70 from the suture holder 766 and active release from the suture holder 766 can be when the user removes the suture 70 from the suture holder 766, for example, by pulling the suture 70 with their hand or with something other than the loader control 762 (e.g., with the device 188). For example, when the shuttle 14 is fully loaded into the device 188 (e.g., when the shuttle 14 is in any position ranging from the shuttle second position to the shuttle third position and the loader control 762 is in any position ranging from the loader control second position to the loader control third position), the user can release the device 188 from the loader 750 and can pull the rest of the suture 70 off of the suture holder 766 (e.g., through the loader control 762), for example, directly with their hands or simply by pulling the device 188 and the loader 750 away from each other.

FIG. 18 illustrates that the suture holder 766 can have ribs 792 that can engage with rib engagers 794. The loader 750 can have, for example, 1 to 3 or more rib engagers 794. For example, FIG. 18 illustrates that the loader 750 can have three rib engagers 794, spaced 120 degrees apart from each other. The ribs and rib engagers 792, 794 can inhibit or prevent the suture holder 766 from moving in direction 766a and/or in direction 766b before use (e.g., when packaged). The ribs and rib engagers 792, 794 can provide tactile and/or audible feedback to the user that can indicate that the suture holder 766 is rotating in direction 766a during loading. The audible feedback can be, for example, audible clicks that are generated as the suture holder rotates and the ribs 792 rotate past the rib engagers 794.

FIG. 18 illustrates that the suture holder 766 can be a spool.

FIG. 18 illustrates that the track 775 on the loader control second side 762_{SS} can have the arrangement shown, for example, that it can extend across the outer surface of the loader control second side 762_{SS}.

As the loader control 762 is moved from the loader control first position to the loader control second position or to the loader control third position, the suture 70 can, for example, passively release (e.g., unspool) from the suture holder 766.

The loader 750 can be packaged with or without suture 70 on (e.g., wound around) the suture holder 766. For example, FIG. 18 illustrates that the loader 750 can be packaged with the suture 70 wound around the suture holder 766. Where the loader 750 is not packaged with the suture 70 attached to the loader 750, the suture 70 can be attached to the suture holder and extended through the loader 762 before use.

FIG. 19A illustrates that the loader control 762 can have the arrangement of features shown. FIG. 19A illustrates that the suture holder 795 can be, for example, a friction member that can inhibit or prevent the suture 70 from slipping through the loader control 762 until the loader control threshold force is reached or exceeded. FIG. 19A illustrates that the suture holder 795 can have a pad 796, a hinge 797, or both the pad 796 and the hinge 797. The suture 70 can pass through the pad 796, over the pad 796, or both. The pad 796 can be one, two or more pads. The hinge 797 can be, for example, a living hinge. The hinge 797 can be, for example, a portion (e.g., a middle portion, a folded portion) of the pad 796. The pad 796 can have the hinge 797. For example, FIG. 19A illustrates that the hinge 797 can be a portion (e.g., a middle portion) of the pad 796. FIG. 19A illustrates that the pad 796 can be foldable, for example, about the hinge 797.

The pad 796 can be, for example, a pad (e.g., a single pad) with two sides (e.g., a pad first side 796a and a pad second side 796b) that can be connected by the hinge 797. FIG. 19A illustrates that when the pad 796 comprises a single pad having two sides (e.g., the pad first and second sides 796a, 796b), the pad 796 can be, for example, folded about the hinge 797 such that the two sides (e.g., the pad first and second sides 796a, 796b) can be pressed into each to releasably hold the suture 70. The fold in the pad 796 can be the hinge 797. As another example (e.g., as an alternative example shown in FIG. 19A), the pad 796 can comprise two pads (e.g., a first pad 777a and a second pad 777b) connected by a hinge (e.g., the hinge 797). FIG. 19A illustrates that when the pad 796 comprise two pads (e.g., first and second pads 777a, 777b), the two pads (e.g., first and second pads 777a, 777b) can be pressed into each other to releasably hold the suture 70. The suture 70 can pass between the two pads of the suture holder 795 (e.g., first and second pads 777a, 777b), or can pass between the two sides (e.g., the pad first and second sides 796a, 796b) of a single pad (e.g., of the pad 796) of the suture holder 795.

The pad first and second sides 796a, 796b can be pressable against each other and the suture 70 to hold the suture 70 as the shuttle 14 is loaded into the device 188 via the loader control 762. The pad first and second sides 796a, 796b can be pressable against each other and the suture 70 to hold the suture 70 as the shuttle 14 is unloaded from the device 188 via the loader control 762. The first and second pads 777a, 777b can be pressable against each other and the suture 70 to hold the suture 70 as the shuttle 14 is loaded into the device 188 via the loader control 762. The first and second pads 777a, 777b can be pressable against each other and the suture 70 to hold the suture 70 as the shuttle 14 is unloaded from the device 188 via the loader control 762.

When the loader control first side 762_{FS} and the loader control second side 762_{SS} are pressed together (e.g., as shown in FIG. 19A), the pad 796 of the suture holder 795 can clamp the suture 70 in the loader control 762 (e.g., either between the pad first and second sides 796a, 796b or between the first and second pads 777a, 777b), inhibiting or preventing the suture 70 from slipping through the loader control 762 until the loader control threshold force is reached or exceeded as the loader control 762 is moved from the loader control first position to the loader control second or third position. The suture 70 can slip through the suture holder 795, for example, as the loader control 762 is moved from a fully loaded position to an overloaded position. For example, the suture 70 can slip through the suture holder 795 as the loader control 762 is moved from the loader control second position to the loader control third position. The loader control threshold can be reached or exceeded by interference between the shuttle 14 and the device 188 as loader control 762 is moved from the loader control second position to the loader control third position. As the loader control 762 is moved from the loader control second position to the loader control third position, the device 188 can allow the shuttle 14 to move from the shuttle second position to the shuttle third position but can resist or interfere with movement of the shuttle 14 further into the device 188 beyond the shuttle third position. For example, as the loader control 762 is moved from the loader control second position to the loader control third position, the shuttle 14 can be moved by the loader control 762 about 0.10 mm to about 3.00 mm further into the device 188, or more narrowly, about 0.10 mm to about 1.50 mm further into the device 188, including every 0.01 mm increment within these ranges (e.g., 0.10 mm, 0.50 mm, 1.50 mm, 3.00 mm). The shuttle 14 can reach the shuttle third position when the loader control 762 is between the loader control second and third positions. As another example, the shuttle 14 can reach the shuttle third position when the loader control 762 reaches the loader control third position. Once the shuttle 14 is in the shuttle third position, further movement of the shuttle 14 into the device 188 can be inhibited or prevented by the device 188, at which point further movement of the loader control 762 to the loader control third position can cause the tension in the suture 70 to increase until the loader control threshold force is reached or exceeded. The loader control threshold force can be reached or exceeded when the loader control 762 is between the loader control second and third positions. The device 188 can inhibit or prevent further movement of the shuttle 14 into the device 188 once the shuttle 14 reaches the shuttle third position, for example, by obstructing further movement of the shuttle 14. Further movement can be inhibited or prevented by the device 188 via a resistor. The resistor can be inside device 188. The resistor can be, for example, the female stop 416 that the male stop 412 can be engaged with when the shuttle 14 is in the shuttle second position, an end of the shuttle track that the shuttle 14 can be in during loading (e.g., the upper jaw track 64 or the jaw lower jaw track 66), or both. For example, the resistor can be a proximal wall or edge of the female stop 416, can be a proximal wall or edge of the shuttle track, or any combination thereof. For example, the shuttle 14 can be considered to be in the shuttle third position when a male stop 412 (e.g., the leading male stop 412) contacts a proximal wall or edge of a female stop 416 (e.g., the wall or edge farthest from the tip of the jaw that the shuttle 14 is in) such that when the shuttle 14 is in the shuttle third position, the engagement between the male stop 412 and the wall or edge of the female stop 416 can inhibit or prevent the shuttle 14 from being loaded further into the device 188 as the loader control 762 is moved further to the loader control third position while the shuttle 14 is in the shuttle third position. As the loader control 762 is moved to the loader control third position, the shuttle 14 can remain in the shuttle third position. This can cause the tension in the suture 70 to increase as the loader control 762 is moved toward the loader control third position, for example, as the distance between the loader control 762 and the shuttle 14 increases. The suture 70 can begin moving through the suture holder 795 (e.g., sliding and/or rolling) once the force on the suture 70 reaches or exceeds the loader control threshold force, at which point the suture 70 can slide and/or roll in the suture holder 795 as the loader control 762 continues to be moved to the loader control third position. The loader control threshold force can be, for example, from about 2.00 lbs to about 10.00 lbs, including every 0.01 lb increment within this range (e.g., 2.00 lbs, 3.00 lbs, 6.00 lbs, 10.00 lbs). The loader control threshold force can be higher than the loading force needed for loading the shuttle 14 into the device 188 or the unloading force need for unloading the shuttle 14 from the device 188. For example, the loading force can be about 0.30 lbs to about 4.00 lbs, including every 0.01 lb increment within this range (e.g., 0.30 lbs, 0.50 lbs, 2.00 lbs, 4.00 lbs). For example, the unloading force can be about 0.30 lbs to about 4.00 lbs, including every 0.01 lb increment within this range (e.g., 0.30 lbs, 0.50 lbs, 2.00 lbs, 4.00 lbs). As another example, the suture 70 can be inhibited or prevented from slipping through the suture holder 795, for example, as the loader control 762 is moved from a fully loaded position to an overloaded position.

FIG. 19A illustrates that the loader control 762 can have a loader control connector 762_{C} that can connect the loader control first and second sides 762_{FS}, 762_{SS}, which can extend through the track 764, which can guide the loader control 762 along the track 764, or any combination thereof.

The loader control 762 can be openable and closable, as shown by the arrows 798a and 798b. FIG. 19A illustrates, for example, that the loader control 762 can be openable and closable about the hinge 797. The loader control first side 762_{FS} can be movable toward the loader control second side 762_{SS} in direction 798a about the hinge 797 to close the loader control 762, to close the suture holder 795, or to close both. The loader control first side 762_{F}s can be movable away from the loader control second side 762_{SS} in direction 798b about the hinge 797 to open the loader control 762, to open the suture holder 795, or to open both. For example, FIG. 19A illustrates that the loader control first side can have a loader control first side first portion 762_{FS1} and a loader control first side second portion 762_{FS2}. The loader control first side second portion 762_{FS2} can be attached to or integral with the loader control second side 762_{SS}, for example, via the loader control connector 762_{C}. The loader control first side first and second portions 762_{FS1}, 762_{FS2} can be first and second portions (e.g., halves) of the loader control first side 762_{FS}. The loader control first side first portion 762_{FS1} can be movable toward the loader control first side second portion 762_{FS2} and toward the loader control second side 762_{SS} in direction 798a about the hinge 797 to close the loader control 762, to close the suture holder 795, or to close both. The loader control first side first portion 762_{FS1} can be movable away from the loader control first side second portion 762_{FS2} and away from the loader control second side 762_{SS} in direction 798b about the hinge 797 to open the loader control 762, to open the suture holder 795, or to open both. For example, the loader control first side first portion 762_{FS1} can be lowered in direction 798a and lifted in direction 798b to close and open the loader control 762, to close and open the suture holder 795, or to close and open both.

As the suture holder 795 is opened, the loader control first side first portion 762_{FS1} can rotate about the hinge 797 away from the loader control first side second portion 762_{FS2}. The suture holder 795 can be opened, for example, to load the loader control 762 with the suture 70, to remove the suture 70 from the loader control 762 (e.g., after the shuttle 14 is fully loaded into the device 188), or both. As the suture holder 795 is closed, the loader control first side first portion 762_{FS1} can rotate about the hinge 797 toward from the loader control first side second portion 762_{FS2}. The suture holder 795 can be closed, for example, to secure the suture 70 to the loader control 762 (e.g., before the shuttle 14 is loaded into the device 188), for example, by clamping the suture 70 between the loader control first and second portions 762_{FS1}, 762_{FS2} as the loader control 762 is moved along the track 764, to inhibit or prevent the suture 70 from slipping through the suture holder 795 as the loader control 762 is moved along the track 764, or both.

FIG. 19A illustrates that the pad first and second sides 796a, 796b can be pressable against each other, for example, by pressing the loader control first and second sides 762_{FS}, 762_{SS} together as shown by the two opposing arrows 799. As another example, the pad first side 796a can be pressable against the pad second side 796b (e.g., the top arrow 799 in FIG. 19A) with or without pressing against the pad second side 796b (e.g., the bottom arrow 799 in FIG. 19A). FIG. 19A illustrates that the first and second pads 777a, 777b can be pressable against each other, for example, by pressing the loader control first and second sides 762_{FS}, 762_{SS} together as shown by the arrows 799. As another example, the first pad 777a can be pressable against the pad second side 796b (e.g., the top arrow 799 in FIG. 19A) with or without pressing against the pad second side 796b (e.g., the bottom arrow 799 in FIG. 19A). The loader control first and second sides 762_{FS}, 762_{SS} can be pressable together, for example, using one or more fingers. One finger can be used, for example, if the device 188 is resting against something (e.g., a table). Two or more fingers (e.g., a thumb and one or more other fingers) can be used to squeeze the loader control 762 with on hand.

FIG. 19B illustrates that the suture 70 can extend though the through the suture holder 795. For example, FIG. 19B illustrates that the suture 70 can extend through (e.g., transversely through) the suture holder 795 between the pad first side 796a and the pad second side 796b. As another example, FIG. 19B illustrates that the suture 70 can extend through (e.g., transversely through) the suture holder 795 between the first pad 777a and the second pad 777b.

FIGS. 19A and 19B illustrate, for example, that the loader control 762 can be openable and closable about the hinge 797 such that the loader control 762 can have an openable and closable space for the suture 70. FIGS. 19A and 19B illustrate, for example, that the loader control 762 can be openable and closable about the hinge 797 such that the loader control 762 can have compressible space for the suture 70. FIG. 19B illustrates that when suture holder 795 is closed, the suture 70 can be clamped in the suture holder 795 such that the suture 70 can be inhibited or prevented from moving (e.g., slipping, sliding, rolling) in the suture holder 795 (e.g., between the pad first and second sides 796a, 796b or between the first and second pads 777a, 777b) until, for example, the loader control threshold force is reached and/or exceeded.

FIGS. 19A and 19B illustrate that the suture 70 can be attachable to the loader control 762. FIGS. 19A and 19B illustrate that the suture 70 can be placeable in the loader control 762. For example, FIGS. 19A and 19B illustrate that the suture 70 can be placeable in suture holder 795. FIGS. 19A and 19B illustrate that the suture 70 can be removable from the loader control 762, for example, removable from the suture holder 795.

The suture holder 795 can hold the suture 70 and/or the suture 70 can slip through the suture holder 795 without the suture 70 being damaged.

The shuttle 14 can be loaded into the device 188 (e.g., into the upper jaw 30 or the lower jaw 38) using one hands or two hands. For example, for two handed loading, one hand can be squeezing the jaw control 8 to clamp the jaws against the loader 750 (e.g., against the cap 758 and/or against the loader body 751) and the other hand can squeeze the loader control 762 (e.g., arrows 799) and move the loader control 762, for example, from the loader control first position to the loader control second position or any subsequent loader control position (e.g., to the loader control third position) in direction 765. For one handed loading, the device 188 can be placed in the device space 752 and the loader 750 can be tilted so that gravity can cause the upper and lower jaws 30, 38 to make contact with the cap 758 and/or with the loader body 751 so that the shuttle 14 can be loaded into the device 188. While the loader 750 is tilted, one or both hands can move the loader control 762, for example, from the loader control first position to the loader control second position or any subsequent loader control position (e.g., to the loader control third position).

The loader 750 can be a single-use loader or a multiple-use loader. Where the loader 750 is a single-use loader, the loader 750 can be disposed of after the shuttle 14 is loaded into the device 188. Where the loader 750 is a multiple-use loader, a new shuttle 14 or the same shuttle 14 can be inserted into the loader 750 (e.g., into the track 758 and/or the track 760), a new suture 70 can be attached to the shuttle 14 and extended through the loader control 762 (e.g., through the suture holder 795), and/or the new suture 70 can be wound around the suture holder 766.

The device 188 can be a multiple-use device, such that one or multiple shuttles 14 can be loaded and removed from the device 188. For example, where the shuttle 14 has a lifespan of 2 to 10 passes through tissue, the shuttle 14 can be removed from the device 188 once the lifespan of the shuttle 14 expires or after the shuttle 14 no longer adequately moves or cuts through tissue (e.g., whichever is earlier) and a new shuttle 14 can be loaded into the device 188 using the same loader 750 (e.g., if the loader is multiple-use) or a different loader 750 (e.g., if the loader is single-use).

The handle of the device 188 in FIGS. 14B-15B, 16, and 17A is not shown for illustrative purposes only.

When the shuttle 14 is in the shuttle first position (e.g., a shuttle non-loaded position), the shuttle 14 can have a shuttle first radius of curvature. When the shuttle 14 is in the shuttle second position (e.g., a shuttle loaded position), the shuttle 14 can have a shuttle second radius of curvature. The shuttle first and second radius of curvatures can be the same as or different from each other. For example, when different from each other, the shuttle first radius of curvature can be less then or greater than the shuttle second radius of curvature. For example, FIG. 14A illustrates that when the shuttle 14 is in the shuttle first position, the shuttle 14 can have the shuttle first radius of curvature, and FIGS. 16 and 17A illustrate that when the shuttle 14 is in the shuttle second position, the shuttle 14 can have the shuttle second radius of curvature. FIGS. 16 and 17A further illustrate that the shuttle second radius of curvature can be less than the shuttle first radius of curvature. For example, the shuttle second radius of curvature can be, for example, about 0.010 in. to about 0.075 in. less than the shuttle first radius of curvature, including every 0.001 in. increment within this range (e.g., 0.010 in., 0.015 in., 0.075 in.). The shuttle 14 can be a spring. For example, when the shuttle 14 has the shuttle second radius of curvature, the shuttle 14 can be biased to have the shuttle first radius of curvature. For example, when the shuttle 14 has the shuttle second radius of curvature and is in the upper jaw 30 or the lower jaw 38, the shuttle 14 can be biased to push the male stops 412 outward (e.g., radially outward) into the female stops 416 when the shuttle 14 is moved (e.g., translated) into the upper jaw 30 or into the lower jaw 38 from the loader 750 (e.g., via the loader control 762). As another example, the shuttle 14 can have the same radius of curvature (e.g., the shuttle first radius of curvature) before and after being loaded into the device 188. For example, the shuttle 14 can have the same radius of curvature (e.g., the shuttle first radius of curvature) when the shuttle 14 is in the shuttle first position and when the shuttle 14 is in the shuttle second position. The loader control 762 can move the male stops 412 into engagement with the female stops 416, for example, by moving the loader in direction 765 (e.g., from the loader control first position to the loader control second position). As another example, the loader control 762 can move the male stops 412 out of engagement with the female stops 416, for example, by moving the loader in a direction opposite to direction 765 (e.g., from the control second position to the loader control first position), for example, if an adjustment is desired (e.g., realign the shuttle 14 or the device 188 in or on the loader 750).

The loader 750 can be used to load a suture (e.g., the suture 70) and/or a shuttle (e.g., the shuttle 14) into a suture device (e.g., a device 188). The loader 750 can have a suture (e.g., the suture 70) and/or a shuttle (e.g., the shuttle 14) that can be moved (e.g., via the loader control 762) from a non-loaded configuration (e.g., FIG. 14A) to a loaded configuration (e.g., FIGS. 16 and/or FIG. 17A) without damaging the suture or the shuttle, or with causing minimal damage to the suture or the shuttle such that the suture or the shuttle can still be used after the minimal damage is caused during the loading the loading process.

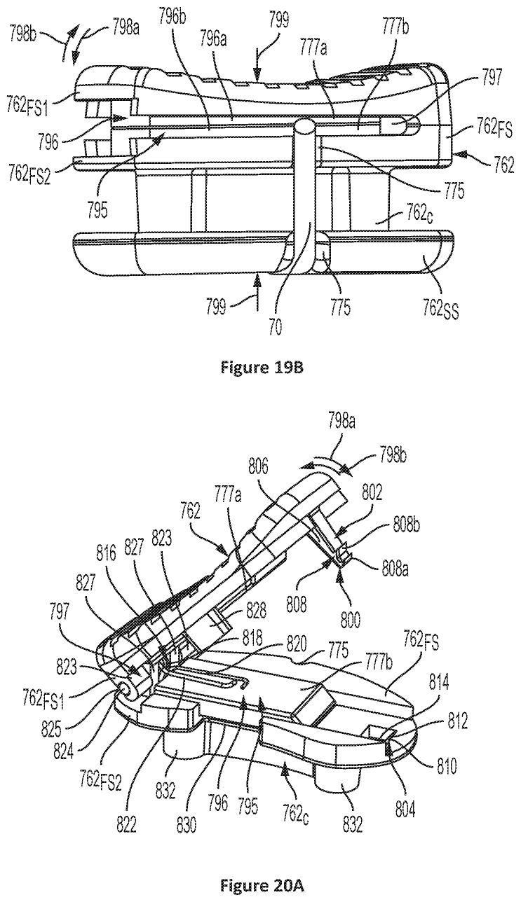

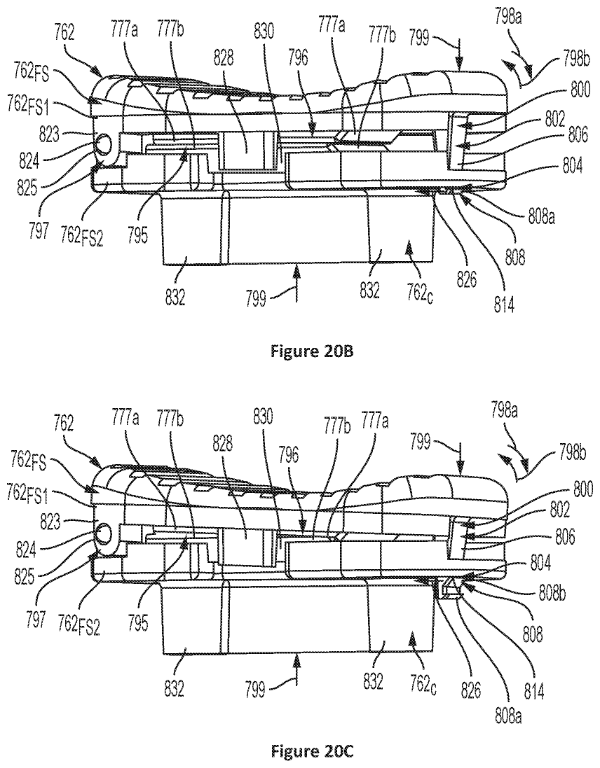

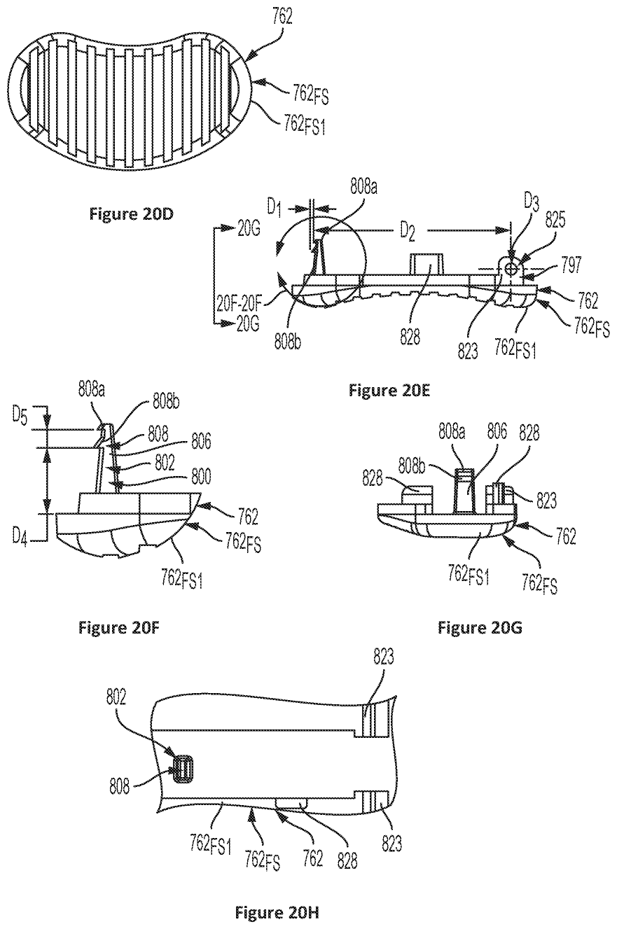

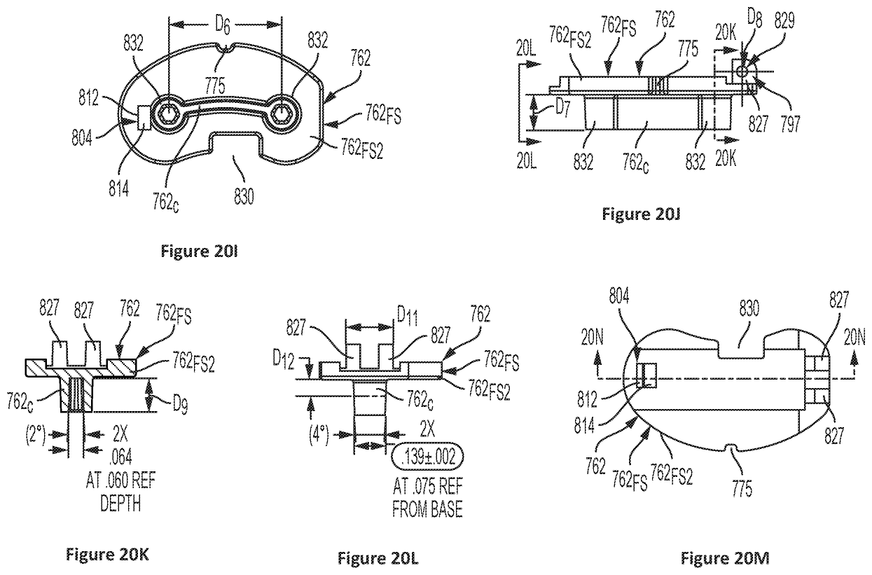

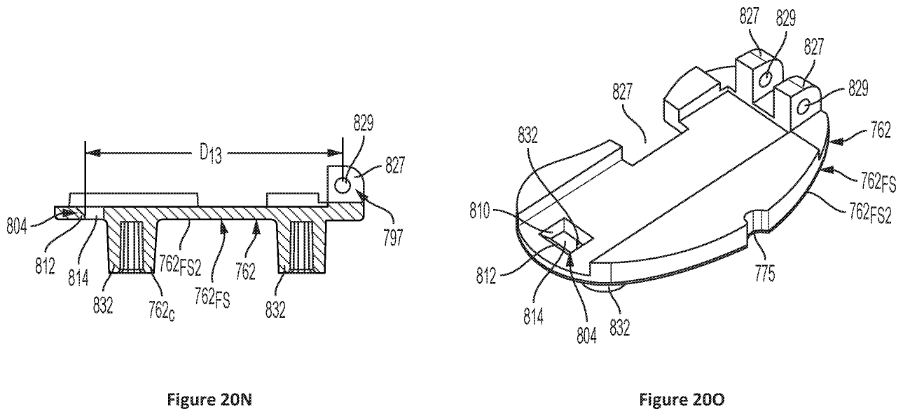

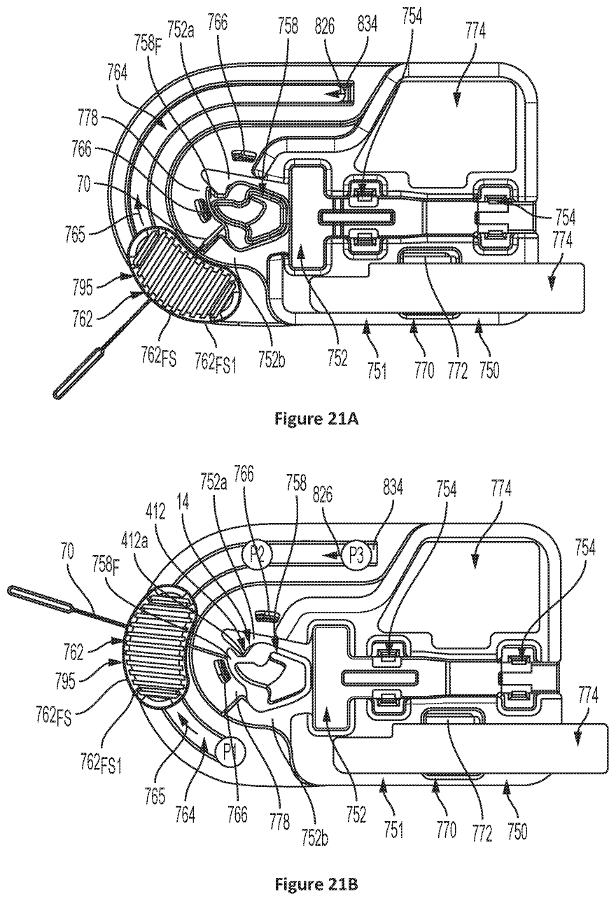

FIG. 20A illustrates that the loader control 762 can be a releasably lockable loader control. FIG. 20A illustrates that the suture holder 795 can be a releasably lockable suture holder. FIG. 20A illustrates that the loader control 762 can have a releasably lockable suture holder (e.g., the suture holder 795). FIG. 20A illustrates that the loader control 762 can have a connector 800. The connector 800 can releasably connect two portions of the loader control 762 together so that the loader control 762 can be openable and closable. The connector 800 can releasably keep the loader control 762 in a closed configuration. The connector 800 can releasably keep the suture holder 795 in a closed configuration. For example, the connector 800 can releasably connect the loader control first side 762_{FS} to the loader control second side 762_{SS}. As another example, the connector 800 can releasably connect the loader control first side first portion 762_{FS1} to the loader control first side second portion 762_{FS2}.

FIG. 20A illustrates that the connector 800 can be, for example, a clip. The clip can be snap. The clip can have a snap fit. The clip can be, for example, a cantilever snap. The clip can be, for example, a latch. The connector 800 can have a male portion 802 and a female portion 804. The female portion 804 can releasably catch the male portion 802. The male portion 802 can have an arm 806 and one or multiple protrusions 808 (e.g., one, two, three, or more protrusions 808). For example, FIG. 20A illustrates that the arm 806 can have a first protrusion 808a and a second protrusion 808b. The protrusions 808 can extend from the arm 806. The first protrusion 808a can be the same size or a different size than the second protrusion 808b. For example, FIG. 20A illustrates that the first protrusion 808a can be smaller than the second protrusion 808b. The female portion 804 can be a catch that can catch the protrusions 808. For example, the female portion 804 can have a tapered entrance 810, a lip 812, and a channel 814, or any combination thereof. The lip 812 can deflect the arm 806 and/or the protrusions 808. The tapered entrance 810 can be part of the channel 814 or may not be part of the channel 814. For example, FIG. 20A illustrates that the channel 814 can have a channel first end and a channel second end, and the tapered entrance 810 can be the channel first end such that the channel first end can be wider than the channel second end. As another example, the female portion 804 may not have the tapered entrance 810. In such cases, the channel first and second ends can have, for example, the same width.

The tapered entrance 814 can align the protrusions 808 with the catch as the loader control 762 is moved from an open configuration to a closed configuration. The tapered entrance 814 can guide the protrusions 808 into the channel 814 as the loader control 762 is moved from an open configuration to a closed configuration. FIG. 20A illustrates, for example, that the tapered entrance 810 can have a proximal tapered surface, a distal tapered surface, and two lateral tapered surfaces, or any combination thereof (e.g., only the distal tapered surface). The lip 812 can be a wall, surface, and/or ledge adjacent to the channel 814 and/or defining the channel 814. For example, FIG. 20A illustrates that the distal wall of the channel 814 (e.g., the distal tapered surface) of the tapered entrance 810 can be the lip 812. For variations that do not have a tapered entrance (e.g., the tapered entrance 810), the lip 812 can be a wall, surface, and/or ledge that is adjacent to and/or defines the channel 814. The channel 814 can be a through-hole in the loader control 962 (e.g., as shown in FIG. 20A), for example, in the loader control first side 962_{FS} (e.g., as shown in FIG. 20A) and/or in the loader control second side 962_{SS}. As another example, the channel 814 can be a recess in the loader control 962, for example, in the loader control first side 962_{FS}. For example, the female portion 804 can be a catch having the channel 814 (e.g., with or without the tapered entrance 810) with a lip 812 that can deflect the arm 806.

The protrusions 808 can be engageable and disengageable with the female portion 804, for example, with the lip 812. For example, the first protrusion 808a can be engageable and disengageable with the lip 812, and the second protrusion 808b can be engageable and disengageable with the lip 812.

The protrusions 808 (e.g., the first and second protrusions 808a, 808b) can be independently engageable and independently disengageable with the female portion 804, for example, with the lip 812. Each of the protrusions 808 (e.g., the first and second protrusions 808a, 808b) can be separately engageable and disengageable with the lip 812.

The protrusions 808 (e.g., the first and second protrusions 808a, 808b) can be sequentially engageable and/or sequentially disengageable with the female portion 804, for example, with the lip 812. For example, as the arrangement of the protrusions 808 in FIG. 20A illustrates, the first protrusion 808a can be engaged with the lip 812, and then the second protrusion 808b can be engaged with the lip 812. As another example, as the arrangement of the protrusions 808 in FIG. 20A illustrates, the second protrusion 808b can be disengaged from the lip 812, and then the first protrusion 808a can be disengaged from the lip 812.

The connector 800 can have one or multiple engaged configurations. The one or multiple engaged configurations can be closed configurations of the connector 800. The number of engaged configurations of the connector 800 can correspond to the number of protrusions 808 that the connector 800 has. For example, FIG. 20A illustrates that the connector 800 can have two protrusions 808, for example, the first protrusion 808a and the second protrusion 808b, such that the connector 800 can have two engaged configurations. For example, FIG. 20B illustrates that the connector 800 can have a connector first engaged configuration (also referred to as a connector first closed configuration) when the first protrusion 808a is engaged with the female portion 804, and FIG. 20C illustrates that the connector 800 can have a connector second engaged configuration (also referred to as a connector second closed configuration) when the second protrusion 808b is engaged with the female portion 804. The engaged configurations can be, for example, clipped or latched configurations.

The male portion 802 can be attached to or integrated with the loader control first side 762_{FS} or the loader control second side 762_{SS}. The female portion 804 can be attached to or integrated with the loader control first side 762_{FS} or the loader control second side 762_{SS}. For example, the male portion 802 can be attached to or integrated with the loader control first side 762_{FS} and the female portion 804 can be attached to or integrated with the loader control second side 762_{SS}, or vice versa. As another example, the male portion 802 and the female portion 804 can both be attached to or integrated with the loader control first side 762_{FS} or the loader control second side 762_{FS}. For example, FIG. 20A illustrates that the male portion 802 can be attached to or integrated with the loader control first side first portion 762_{FS1} and that the female portion 804 can be attached to or integrated with the loader control first side second portion 762_{FS2}. FIG. 20A illustrates, for example, that the arm 806 can extend from the loader control first side first portion 762_{FS1} and that the channel 814 can extend through the loader control first side second portion 762_{FS2}. As another example, the male portion 802 can be attached to or integrated with the loader control first side second portion 762_{FS2} and the female portion 804 can be attached to or integrated with the loader control first side first portion 762_{FS1} such that the arm 806 can extend from the loader control first side second portion 762_{FS2} and such that the channel 814 can extend through the loader control first side first portion 762_{FS1}.

FIG. 20A illustrates that the loader control 762 can have a spring 816. The spring 816 can be, for example, a compression spring or a torsion spring. For example, FIG. 20A illustrates that the spring 816 can be a torsion spring having a coil 818, a spring first arm 820, and a spring second arm 822. The spring 816 can bias the loader control 762 to have an open configuration (e.g., the open configuration shown in FIG. 20A). The spring 816 can bias the connector 800 to have an open configuration (e.g., the open configuration shown in FIG. 20A). The spring 816 can be metal, for example, steel.

FIG. 20A illustrates that the hinge 797 may not be a living hinge. For example, FIG. 20A illustrates that the hinge 797 can have a pin 824 and that the hinge 797 can be loaded with the spring 816. The pin 824 can, for example, extend through the coil 818. The pin 824 can, for example, extend through one or more arms 823 of the loader control first side first portion 762_{FS1}. FIG. 20A illustrates that the loader control first side first portion 762_{FS1} can have two arms 823, each with a channel 825. The pin 824 can extend through the channels 825. The pin 824 can, for example, extend through one or more arms 827 of the loader control first side second portion 762_{FS2}. FIG. 20A illustrates that the loader control first side second portion 762_{FS2} can have two arms 827, each with a channel 829 (shown in FIG. 20J). The pin 824 can extend through the channels 829. The hinge 797 can include, for example, the arms 823, the channels 825, the arms 827, the channels 829, the spring 816, the pin 824, or any combination thereof. As another example, the hinge 797 can be a living hinge such as the hinge 797 shown in FIGS. 19A and 19B.

In FIG. 20A, the spring first and second arms 820, 822 are both shown on the second pad 777b, for example, so that both the spring arms can be clearly seen. However, in operation, the spring first arm 820 can be biased against the first pad 777a and the spring second arm 822 can be biased against the second pad 777b such that as the loader control 762 is closed from an open configuration (e.g., the open configuration shown in FIG. 20A), the spring first and second arms 820, 822 can resist the closure by pressing against the first and second pads 777a, 777b, respectively. In other words, in operation, the spring first arm 820 can be in contact with the first pad 777a and the spring second arm 822 can be in contact with the second pad 777b when the loader control 762 is in an open configuration (e.g., the open configuration shown in FIG. 20A). The open configuration shown in FIG. 20A can be, for example, a partially open configuration (e.g., a half open configuration). The open configuration shown in FIG. 20A can be, for example, a fully open configuration.

FIGS. 20A-20C illustrate that the protrusions 808 (e.g., the first and second protrusions 808a, 808b) can be engaged with the female portion 804 by closing the loader control 962. The loader control 962 can be closed, for example, by moving (e.g., rotating, translating, or both rotating and translating) the loader control first side 762_{FS} (e.g., the loader control first side first portion 762_{FS1}) toward the loader control second side 762_{SS}. For example, FIG. 20A illustrates that the loader control 962 can be closed by moving (e.g., rotating) the loader control first side 762_{FS} (e.g., the loader control first side first portion 762_{FS1}) toward the loader control second side 762_{SS} about the hinge 797 in direction 798a. As another example, FIG. 20A illustrates that the loader control 962 can be closed by moving (e.g., rotating, translating, or both rotating and translating) the loader control first side first portion 762_{FS1} toward the loader control first side second portion 762_{FS2} about the hinge 797 in direction 798a. As yet another example, FIG. 20A illustrates that the loader control 962 can be closed by moving (e.g., rotating, translating, or both rotating and translating) the loader control first side first portion 762_{FS1} toward the loader control first side second portion 762s2 and toward the loader control second side 762_{SS} about the hinge 797 in direction 798a.

FIGS. 20B and 20C illustrate that the male portion 802 can be moved into (e.g., pushed into) the female portion 804. FIGS. 20B and 20C illustrate that the male portion 802 can be moved into (e.g., pushed into) engagement with the female portion 804. FIG. 20B illustrates, for example, that when the first protrusion 808a is engaged with the female portion (e.g., with the lip 812), the loader control 962 can have a loader control first engaged configuration (also referred to as a loader control first closed configuration). The loader control first closed configuration can be a releasably lockable first closed configuration. The loader control 762 can have the loader control first engaged configuration when the connector 800 has the connector first engaged configuration. FIG. 20C illustrates, for example, that when the second protrusion 808b is engaged with the female portion 804 (e.g., with the lip 812), the loader control 962 can have a loader control second engaged configuration (also referred to as a loader control second closed configuration). The loader control first closed configuration can be a releasably lockable second closed configuration. The loader control 762 can have the loader control second engaged configuration when the connector 800 has the connector second engaged configuration.

FIGS. 20B and 20C illustrate that the loader control 762 can be releasably lockable with the connector 800. The loader control 762 can be, for example, releasably locked by engaging the male portion 802 with the female portion 804 or vice versa. The loader control 762 can be, for example, unlocked from a releasably locked configuration (e.g., from the releasably lockable first closed configuration and/or from the releasably lockable second closed configuration) by disengaging the male portion 802 from the female portion 804 or vice versa.

FIGS. 20A and 20B illustrate that the first protrusion 808a can be moved into engagement with the lip 812, for example, by the user moving (e.g., pushing) the loader control first side 762_{FS} (e.g., the loader control first side first portion 762_{FS1}) toward the loader control second side 762_{SS} about the hinge 797 in direction 798a. FIGS. 20A and 20B illustrate that the first protrusion 808a can be moved into engagement with the lip 812, for example, by the user moving (e.g., pushing) the loader control first side first portion 762_{FS1} toward the loader control first side second portion 762_{FS2} about the hinge 797 in direction 798a. FIGS. 20A and 20B illustrate that the first protrusion 808a can be moved into engagement with the lip 812, for example, by the user moving (e.g., pushing) the loader control first side first portion 762_{FS1} toward the loader control first side second portion 762_{FS2} and toward the loader control second side 762_{SS} about the hinge 797 in direction 798a. One or both sides of the loader control 762 can be moved toward the opposite side of the loader control 762 as shown by the two opposing arrows 799 in FIG. 20B.

FIGS. 20A and 20B illustrate that the user can move the loader control 762 from an open configuration to a first closed configuration (e.g., from the open configuration in FIG. 20A to the closed configuration in FIG. 20B), for example, by forcing the first protrusion 808a into the female portion 804 and/or by forcing the female portion 804 over the first protrusion 808a. This can be accomplished, for example, by applying a compressive force against the loader control 762 as shown by the two opposing arrows 799 in FIG. 20B. A compressive force can be applied to the loader control 762, for example, by the user squeezing two sides of the loader control 762 (e.g., the loader control first and second sides 762_{FS}, 762_{SS}) together until they hear an audible click, which can indicate that the first protrusion 808a is engaged with the lip 812. The audible click can be generated, for example, by the arm 806 snapping against the lip 812. The arm 806 can snap against the lip 812, for example, once the first protrusion 808a is moved past the lip 812.

FIGS. 20A-20C illustrate that the second protrusion 808b can be moved into engagement with the lip 812, for example, by the user moving (e.g., pushing) the loader control first side 762_{FS} (e.g., the loader control first side first portion 762_{FS1}) toward the loader control second side 762_{SS} about the hinge 797 in direction 798a. One or both sides of the loader control 762 can be moved toward the opposite side of the loader control 762 as shown by the two opposing arrows 799 in FIGS. 20B and 20C.

FIGS. 20A-20C illustrate that the user can move the loader control 762 from an open configuration to a second closed configuration (e.g., from the open configuration shown in FIG. 20A to the closed configuration shown in FIG. 20C), for example, by forcing the second protrusion 808b into the female portion 804 and/or by forcing the female portion 804 over the second protrusion 808b. This can be accomplished, for example, by applying a compressive force against the loader control 762 as shown by the two opposing arrows 799 in FIGS. 20B and 20C. A compressive force can be applied to the loader control 762, for example, by the user squeezing two sides of the loader control 762 (e.g., the loader control first and second sides 762_{FS}, 762_{SS}) together until they hear two sequential clicks, the first of which can indicate that the first protrusion 808a is engaged with the lip 812 and the second of which can indicate that the second protrusion 808b is engaged with the lip 812. The first audible click can be generated, for example, by the portion of the arm 806 between the first protrusion 808a and the second protrusion 808b snapping against the lip 812, and the second audible click can be generated, for example, by the portion of the arm 806 between the second protrusion 808b and the base of the arm 806 snapping against the lip 812. The first audible click can be generated, for example, once the first protrusion 808a is moved past the lip 812, and the second audible click can be generated, for example, once the second protrusion is moved past the lip 812.

FIGS. 20A-20C illustrate that the user can move the loader control 762 from the loader control first closed configuration to the loader control second closed configuration (e.g., from the closed configuration shown in FIG. 20B to the closed configuration shown in FIG. 20C), for example, by forcing the male portion 802 of the connector 800 further into the female portion 804 of the connector 800, or vice versa, such that the second protrusion 808b can become engaged with the female portion 804. This can be accomplished, for example, by applying a compressive force against the loader control 762 when the loader control 762 is in the loader control first closed configuration as shown by the two opposing arrows 799 in FIG. 20B. The arrows 799 in FIG. 20C illustrate that the user can choose to compress the loader control 762 further for an even a stronger grip on the suture 70, for example, between the first pad 777a and the second pad 777b if desired. A compressive force can be applied to the loader control 762 when the loader control 762 is in the loader control first closed configuration, for example, by the user squeezing two sides of the loader control 762 (e.g., the loader control first and second sides 762_{FS}, 762_{SS}) together until they hear an audible click, which can indicate that the second protrusion 808b is engaged with the lip 812. The audible clicks can be generated, for example, by the arm 806 snapping against the lip 812.

The loader control first closed configuration can be the shipping configuration of the loader control 762. The loader control first closed configuration can be the packaged configuration of the loader control 762. For example, the loader 750 can be packaged with the loader control 762 in the loader control first closed configuration. As another example, an open configuration (e.g., the open configuration shown in FIG. 20A) can be the shipping configuration of the loader control 762 such that the loader 750 can be packaged with the loader control 762 in an open configuration.

The loader control second closed configuration can be the shuttle loading configuration of the loader control 762. For example, the loader control 762 can have the loader control second closed configuration when the shuttle 14 is loaded into the device 188 as the loader control 762 is moved along the track 764, for example, in direction 765. For example, the loader control 762 can have the loader control second closed configuration as the shuttle 14 is loaded into the device 188 as the loader control 762 is moved along the track 764, for example, in direction 765 from a first position along the track 764 (e.g., from the loader control first position) to a second position along the track 764 (e.g., to the loader control second position).

The loader control second closed configuration can be the shuttle unloading configuration of the loader control 762. For example, the loader control 762 can have the loader second closed configuration when the shuttle 14 is unloaded from the device 188 as the loader control 762 is moved along the track 764, for example, in a direction opposite to direction 765. For example, the loader control 762 can have the loader control second closed configuration as the shuttle 14 is unloaded from the device 188 as the loader control 762 is moved along the track 764, for example, in a direction opposite to direction 765 from a second position along the track 764 (e.g., from the loader control second position) to a first position along the track 764 (e.g., to the loader control first position).

The loader control first closed configuration (e.g., FIG. 20B) can be a lighter hold than the loader control second closed configuration (e.g., FIG. 20C). For example, the loader control first closed configuration can clamp the suture 70 less than the loader control second closed configuration. For example, when the loader control 762 is in the loader control first closed configuration, the suture 70 may not be clamped by the first and second pads 777a, 777b or the suture 70 can be clamped by the first and second pads 777a, 777b but the suture 70 may not be inhibited from slipping through the loader control 762 (e.g., from slipping between the first and second pads 777a, 777b) as the loader control 762 is moved along the track 764. When the loader control 762 is in the loader control second closed configuration, the suture 70 can be clamped by the first and second pads 777a, 777b such that the suture 70 can be inhibited or prevented from slipping through the loader control 762 (e.g., from slipping between the first and second pads 777a, 777b) as the loader control 762 is moved along the track 764 until the loader control threshold force is reached or exceeded.

FIG. 20B illustrates that when the loader control 762 is in the loader control first closed configuration, the first and second pads 777a, 777b may not contact each other. In other words, a gap can be between the first and second pads 777a, 777b when the loader control 762 is in the loader control first closed configuration. As another example, there may not be a gap between the first and second pads 777a, 777b when the loader control 762 is in the loader control first configuration such that the first and second pads 777a, 777b can contact each other when the loader control 762 is in the loader control first closed configuration.

FIG. 20C illustrates that when the loader control 762 is in the loader control second closed configuration, the first and second pads 777a, 777b can contact each other.

The first and second pads 777a, 777b can be a compressible material. The first pad 777a can be a compressible material, the second pad 777b can be a compressible material, or the first pad 777a and the second pad 777b can be a compressible material. The first pad 777a can be compressible, the second pad 777b can be compressible, or the first pad 777a and the second pad 777b can be compressible. The first pad 777a can be incompressible, the second pad 777b can be incompressible, or the first pad 777a and the second pad can be incompressible. As another example, the first pad 777a can be compressible and the second pad 777b can be incompressible, or vice versa. The first pad 777a and the second pad 777b can have the same hardness or a different hardness.

FIG. 20B illustrates that when the loader control 762 is in the loader control first closed configuration, the first and second pads 777a, 777b may not compressed. As another example, when the loader control 762 is in the loader control first closed configuration, the first and second pads 777a, 777b can be compressed less than when the connector 800 is in the connector second closed configuration.

FIG. 20C illustrates that when the loader control 762 is in the loader control second closed configuration, the first and second pads 777a, 777b can be pressed against each other such that the first and second pads 777a, 777b are in a compressed state. This compression can inhibit or prevent the suture 70 from slipping through the loader control 762 as the loader control 762 is moved along the track 764 until the loader control threshold force is reached or exceeded.

The loader control first and second closed configurations can be advantageous, for example, for both shipping and use purposes. The loader control first closed configuration can, for example, inhibit or prevent the pad 796 (e.g., the first pad 777a and the second pad 777b, the pad first side 796a and the pad second side 796b) from contacting each other or being compressed before use so that the lifespan of the pad 796 (e.g., the first pad 777a and the second pad 777b, the pad first side 796a and the pad second side 796b) can be preserved. This can help ensure that the first and second pads 777a, 777b or the pad first and second sides 796a, 796b can inhibit or prevent the suture 70 from slipping through the loader control 762 as the loader control 762 is moved along the track 764. The loader control second closed configuration can, for example, indicate to the user that the loader control 762 has been closed far enough to adequately pinch the suture 70 during movement of the loader control 762 to load and unload the shuttle 14 and/or the suture 70. As another example, the loader control second closed configuration can allow the user to let go of the loader control 762 or relax their grip on the loader control 762 without the suture 70 slipping through the loader control 762, for example, because the clip 800 can keep the loader control 762 in the loader control second closed configuration. This can allow the user to focus on moving the loader control 762 to load and/or unload the shuttle 14 without also having to keep squeezing the loader control 762. This can make the loader control 750 easier and less complicated to use and can reduce the likelihood of the suture 70 prematurely slipping through the loader control 762 during loading or unloading.

FIGS. 20A-20C illustrate that the protrusions 808 (e.g., the first and second protrusions 808a, 808b) can be disengaged from the female portion 804 by opening the loader control 962 from a closed configuration. The loader control 962 can be opened, for example, by disengaging the male portion 802 from the female portion 804 and moving (e.g., rotating, translating, or both rotating and translating) the loader control first side 762_{FS} (e.g., the loader control first side first portion 762_{FS1}) away from the loader control second side 762_{SS}. The loader control 962 can be opened, for example, by disengaging the male portion 802 from the female portion 804 and moving (e.g., rotating, translating, or both rotating and translating) the loader control first side first portion 762_{FS1} away from the loader control first side second portion 762_{FS2}. For example, FIGS. 20B and 20C illustrate that the loader control 962 can be opened by disengaging the male and female portions 802, 804 and moving (e.g., rotating) the loader control first side 762_{FS} (e.g., the loader control first side first portion 762_{FS1}) away from the loader control second side 762_{SS} about the hinge 797 in direction 798b. The loader control 962 can be opened from a closed configuration (e.g., from the loader control first closed configuration or the loader control second closed configuration) to an open configuration (e.g., to the open configuration shown in FIG. 20A). The loader control 962 can be opened, for example, by disengaging the male portion 802 from the female portion 804, whereby once the male portion 802 is disengaged from the female portion 804, the spring 816 can move (e.g., rotate, translate, or both rotate and translate) the loader control first side 762_{FS} (e.g., the loader control first side first portion 762_{FS1}) away from the loader control second side 762_{SS} to an open configuration (e.g., the open configuration shown in FIG. 20A). The loader control 962 can be opened, for example, by disengaging the male portion 802 from the female portion 804, whereby once the male portion 802 is disengaged from the female portion 804, the spring 816 can move (e.g., rotate, translate, or both rotate and translate) the loader control first side first portion 762_{FS1} away from the loader control first side second portion 762_{FS2} to an open configuration (e.g., the open configuration shown in FIG. 20A). For example, FIGS. 20B and 20C illustrate that the loader control 962 can be opened by disengaging the male and female portions 802, 804, whereby once the male and female portions 802, 804 are disengaged from each other, the spring 816 can move (e.g., rotate) the loader control first side 762_{FS} (e.g., the loader control first side first portion 762_{FS1}) away from the loader control second side 762_{SS} about the hinge 797 in direction 798b. As another example, FIGS. 20B and 20C illustrate that the loader control 962 can be opened by disengaging the male and female portions 802, 804, whereby once the male and female portions 802, 804 are disengaged from each other, the spring 816 can move (e.g., rotate) the loader control first side first portion 762_{FS1} away from the loader control first side second portion 762_{FS2} about the hinge 797 in direction 798b.

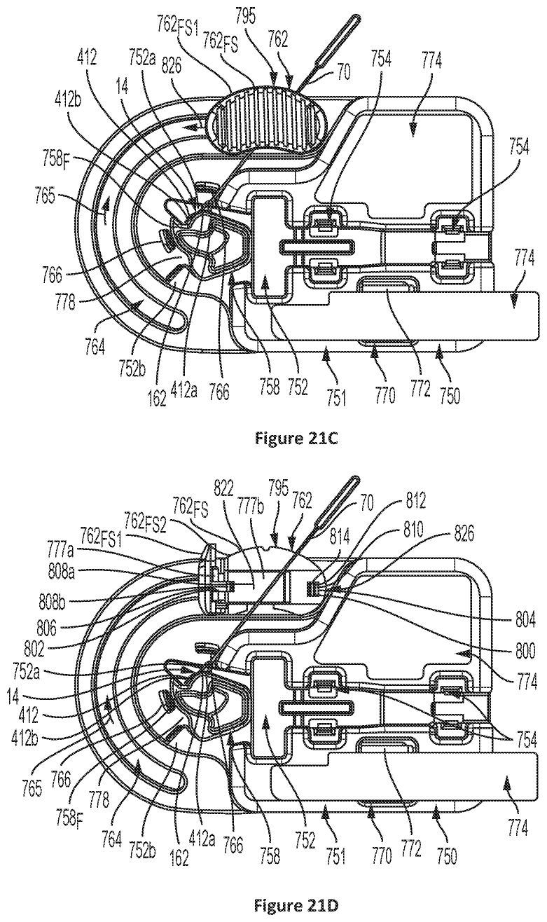

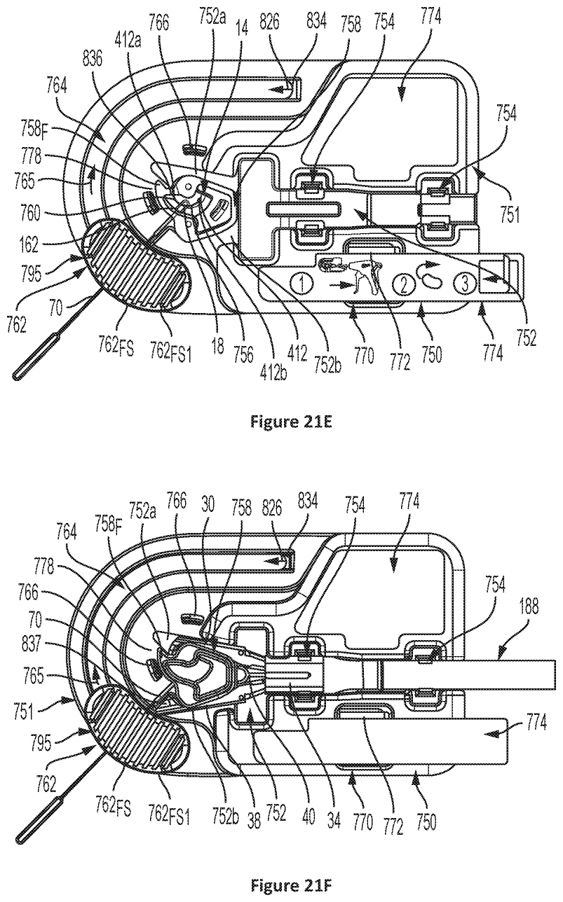

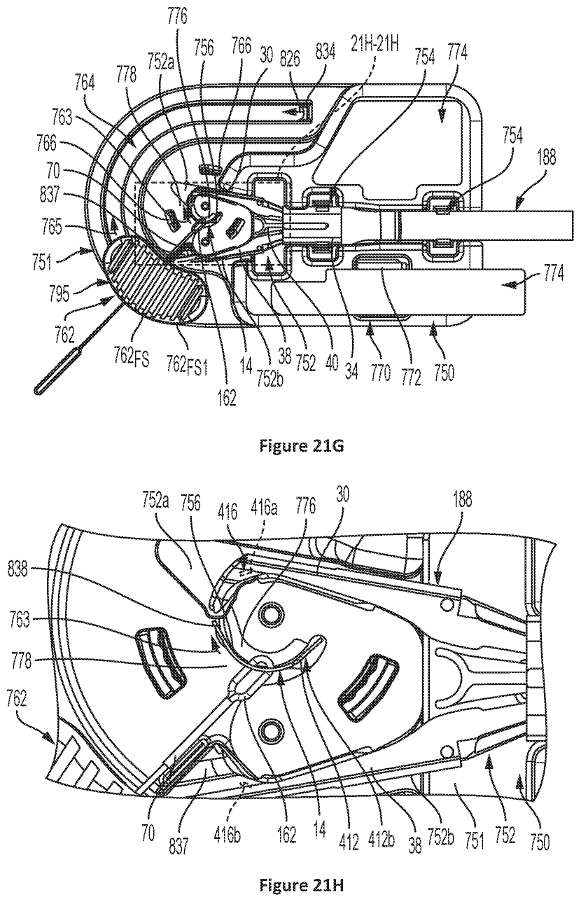

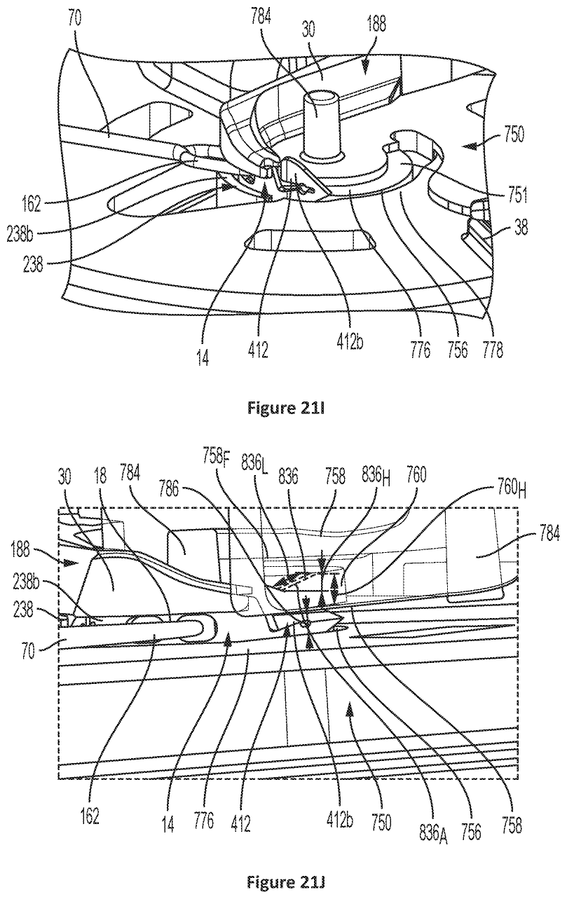

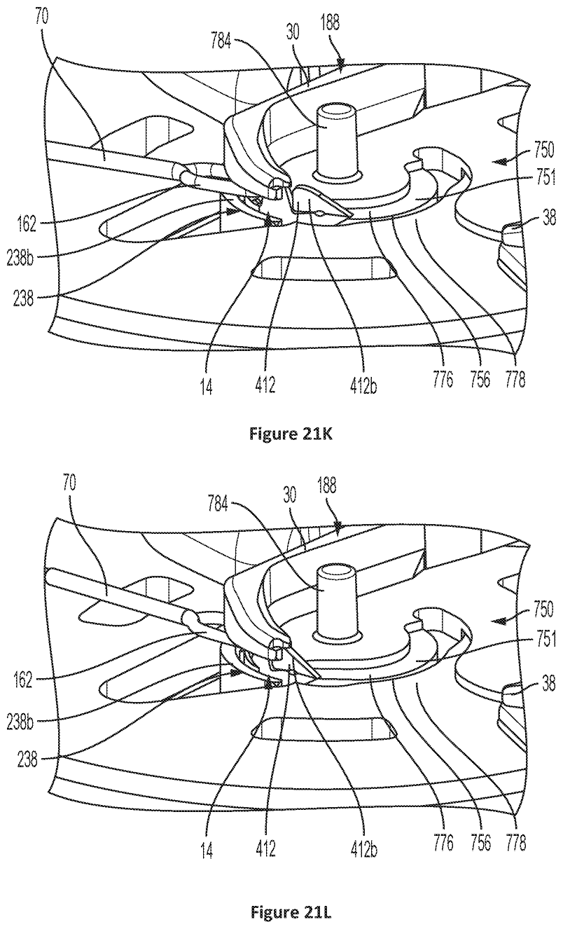

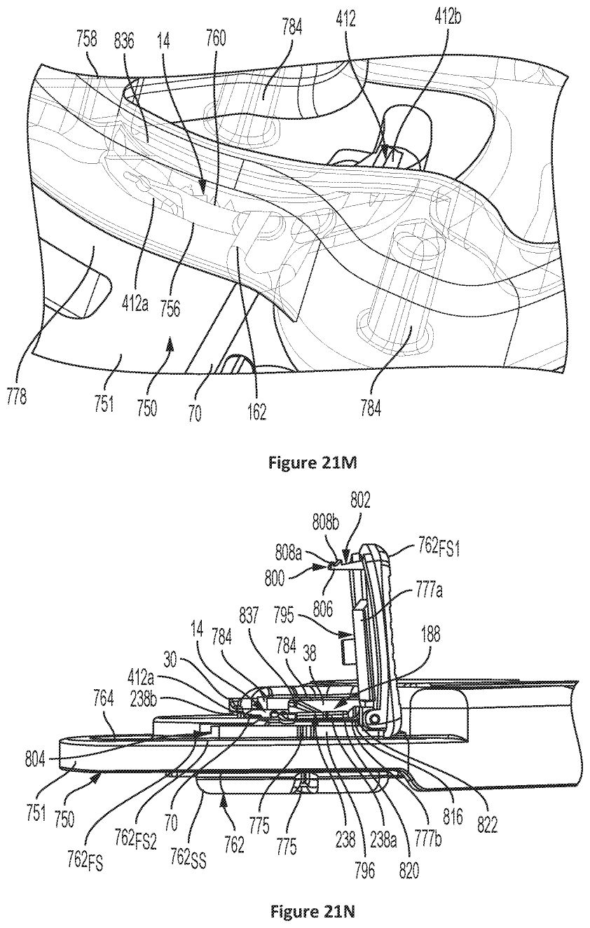

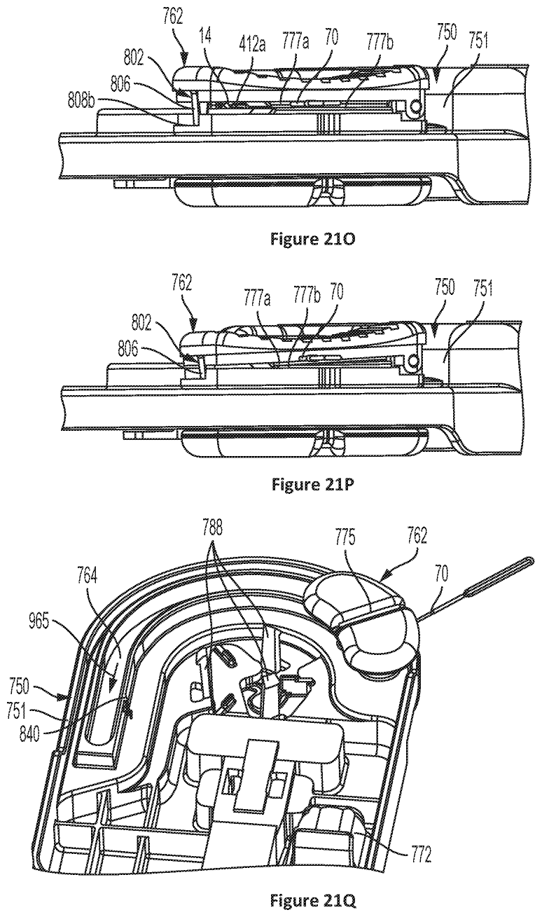

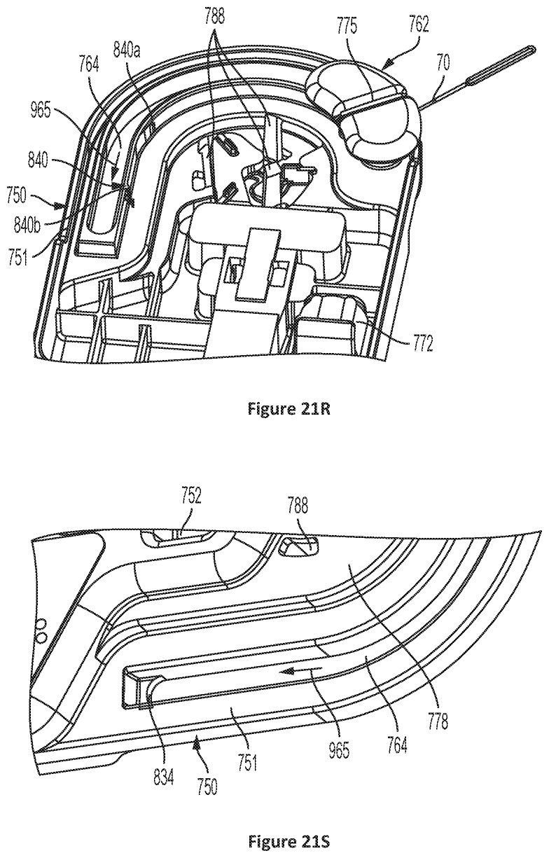

FIGS. 20B and 20C illustrate that the male portion 802 can be disengaged from the female portion 804 by deflecting the arm 806 and the protrusions 808 in direction 826. Direction 826 can be a direction opposite the protrusions 808. For example, FIGS. 20B and 20C illustrate that direction 826 can be toward the hinge 797. The user can deflect the arm and the protrusions 808 in direction 826, for example, by pushing the arm 806 and one or both of the protrusions 808 in direction 826. In such a case, the loader control 762 can be actively opened from a closed configuration by the user. As another example, the arm 806 and/or the protrusions 808 can be deflectable in direction 826 by the loader 750 (e.g., by the opener 834 shown in FIG. 21A) when the loader control 762 reaches the loader control second or third position or any position between the loader control second and third positions. In such a case, the loader control 762 can be passively opened from a closed configuration by the loader 750.

Once the protrusion 808 (e.g., the first protrusion 808a or the second protrusion 808b) that is engaged with the female portion 804 is deflected far enough to clear the lip 812, the spring 816 and/or the user can open the loader control 762 to an open configuration. For example, FIG. 20B illustrates that when the first protrusion 808a is engaged with the lip 812, the arm 806 and/or one or both protrusions 808 can be deflected (e.g., pushed) in direction 826, and once the leading edge of the first protrusion 808a clears the lip 812, the spring 816 and/or the user can open the loader control 762 from the loader control first closed configuration to an open configuration (e.g., to the open configuration shown in FIG. 20A). As another example, FIG. 20C illustrates that when the second protrusion 808b is engaged with the lip 812, the arm 806 and/or one or both protrusions 808 can be deflected (e.g., pushed) in direction 826, and once the leading edge of the second protrusion 808a clears the lip 812, the spring 816 and/or the user can open the loader control 762 from the loader control second closed configuration to the loader control first closed configuration or directly from the loader control second closed configuration to an open configuration (e.g., to the open configuration shown in FIG. 20A).

FIGS. 20A-20C illustrate that the loader control 762 can be operated with one finger (e.g., with the thumb or any other finger). For example, the loader control 762 can be a thumb button. For example, the loader control 762 can be a thumb slide.

FIGS. 20A-20C illustrate that the loader control 762 can have a tab 828 and a space 830 for the tab 828 to slide into and out of as the loader control 762 is opened and closed. FIGS. 20A-20C illustrate that the loader control first side first portion 762_{FS1} can have the tab and that the loader control first side second portion 762_{FS2} can have the space 830. The space 830 can be, for example, a groove in the side of the loader control 762. FIG. 20B illustrates that the tab 828 can be in the space 830 when the loader control 762 is in the loader control first closed configuration. FIG. 20C illustrates that the tab 828 can be in the space 830 when the loader control 762 is in the loader control second closed configuration. The tab 828 can keep the suture 70 aligned (e.g., centered) over the pad 796 (e.g., over the middle of the first and second pads 777a, 777b, over the middle of the pad first and second sides 796a, 796b) so that the pad 796 can maintain friction on the suture 70. The tab 828 can keep the suture 70 from interfering with the spring first and second arms 820, 822 or vice versa. The tab 828 can inhibit or prevent the suture 70 from contacting the spring 816 (e.g., the spring first and second arms 820, 822), for example, by inhibiting or preventing the suture 70 from migrating into contact with the spring 816 (e.g., with the spring first and second arms 820, 822) when the loader control 762 is moved, for example, along the track 764. The tab 828 can be a barrier that can inhibit or prevent the suture 70 from moving (e.g., migrating, rolling, slipping, sliding) toward the spring 816 when the loader control 762 is moved along the track 764 when the loader control 762 is in a closed configuration (e.g., the loader control first and/or second closed configurations). For example, the tab 828 can inhibit or prevent the suture 70 from slipping away from a center of the pad 796 as the loader control 762 is moved. The tab 828 can keep the suture 70 centered over the middle of the pad 796 (e.g., centered over the first and second pads 777a, 777b, centered over the pad first and second sides 796a, 796b) as the loader control 762 is moved in direction 765 away from the loader control first position and/or as the loader control 762 is moved in a direction opposite direction 765 toward the loader control first position. The pad 796 can have a higher friction than the spring 816, so the tab 828 can inhibit or prevent the suture 70 from contacting the spring 816 (e.g., the spring first and second arms 820, 822) so that the pad 796 can maintain friction on the suture 70. Contact between the suture 70 and the spring first and second arms 820, 822 can damage the suture 70, can cause the suture 70 to lose friction with the pad 796 and slip during loading (e.g., during a high-tension part of the loading process), or both. The tab 828 can inhibit or prevent such contact with the spring 816 (e.g., with the spring first and second arms 820, 822) from occurring. The space 830 can allow the tab 828 to extend past the centerline of the pad 796 (e.g., past the centerline of the first and second pads 777a, 777b) to inhibit or prevent the suture 70 from slipping underneath the tab 828 as the loader control 762 is moved. As another example, the suture 70 can contact the spring 816 (e.g., the spring first and second arms 820, 822) when the loader control 762 is moved. The tab 828 and the space 830 can align the male portion 802 with the female portion as the loader control 762 is moved from an open configuration (e.g., the open configuration shown in FIG. 20A) to a closed configuration (e.g., to the loader control first closed configuration shown in FIG. 20B and/or to the loader control second closed configuration shown in FIG. 20C). When the loader control 762 is in a closed configuration, the tab 828 can obstruct the suture 70 from translating across the pad 796 (e.g., across the first and second pads 777a, 777b, across the pad first and second sides 796a, 796b). When the loader control 762 is in a closed configuration, the tab 828 can compress the suture 70 against the side of the loader control 762. When the loader control 762 is in a closed configuration, the suture 70 can be on either lateral side of the tab 828 in the space 830.

FIGS. 20A-20C illustrate that the ends 832 of the loader control connector 762_{C} can be larger than a center (e.g., a longitudinal center) of the loader control connector 762_{C}. Such an arrangement can reduce the friction between the walls of the track 764 and the loader control connector 762_{C} as the loader control 764 is moved in the track 764. The ends 832 can be larger, for example, to maintain wall thickness and strength of the loader control connector 762_{C} while also accommodating two posts of the mating part (e.g., two posts of the loader second side 762_{SS}). For example, the loader control connector 762_{C} can have two male posts that can press fit into holes (e.g., hexagon shaped holes) in the ends 832 of the loader control connector 762_{C}. Exemplary holes are shown, for example, in FIGS. 20I, 20K, and 20N. FIGS. 20I, 20K, and 20N illustrate that the holes can be centered on the larger ends 832. The ends 832 (e.g., bosses) can provide more of a bearing surface for smooth sliding in the track 764.

In FIGS. 20A-20C, the loader second side 762_{SS} is shown transparent for illustrative purposes only, for example, so that the loader control connector 762_{C} can be easily seen. The loader control second side 762_{SS} can be seen attached to or integrated with the loader control connector 762_{C}, for example, in FIGS. 21A-21S (e.g., see FIGS. 21A-21H and 21N-21R). As another example, the loader control connector 762_{C} can be the loader second side 762_{SS}.

FIGS. 20A-20C illustrate that the loader control 762 can be made of plastic, for example, EMERGE 7700 PC/ABS—BLACK. For example, the loader control first side 762_{FS}, the loader control second side 762_{SS}, and/or the loader control connector 762_{C} can be made of EMERGE 7700 PC/ABS—BLACK.

As another example to the variation of the connector 800 shown in FIGS. 20A-20C, the arm 806 can have a single protrusion (e.g., only the first protrusion 808a or only the second protrusion 808b), and the female portion can have two lips 812, for example, a first lip and a second lip spaced apart from the first lip (e.g., similar to how the first and second protrusions 808a, 808b are spaced apart) such that the single protrusion can engage and disengage with the first and second lips.

FIGS. 20D-20H illustrate that the loader control 762 (e.g., the loader control first side first portion 762_{FS1}) can have the dimensions and the arrangement of features shown. The pad 796 (e.g., the pad first side 796a, the first pad 777a) is shown transparent in FIGS. 20D-20H for illustrative purposes only.

FIG. 20D illustrates that the loader control first side first portion 762_{FS1} can have the shape shown when viewed from above. The shape shown in FIG. 20D can be, for example, a kidney bean shape

FIG. 20E illustrates the arrangement of features shown, for example, with exemplary dimensions D₁-D₃.

FIG. 20E illustrates that dimension D₁ can be the distance (e.g., length) between the leading edge of the first protrusion 808a and the leading edge of the second protrusion 808b. Dimension D₁ can be, for example, from about 0.000 inches to about 0.025 inches, or more narrowly from about 0.003 inches to about 0.013 inches, including every 0.001 inch increment within these ranges (e.g., 0.000 inches, 0.003 inches, 0.008 inches, 0.013 inches, 0.025 inches).

FIG. 20E illustrates that dimension D₂ can be the distance (e.g., length) between the leading edge of the first protrusion 808a and the center of the channel 825. Dimension D₂ can be, for example, from about 0.500 inches to about 1.000 inches, or more narrowly from about 0.669 inches to about 0.773 inches, including every 0.001 inch increment within these ranges (e.g., 0.500 inches, 0.669 inches, 0.771 inches, 0.773 inches, 1.000 inches).

FIG. 20E illustrates that dimension D₃ can be the diameter of the channel 825. Dimension D₃ can be, for example, from about 0.0475 inches to about 0.0485 inches, including every 0.0001 inch increment within this range (e.g., 0.0475 inches, 0.0480 inches, 0.0485 inches).

FIG. 20F illustrates the arrangement of features shown, for example, with exemplary dimensions D₄ and D₅.

FIG. 20F illustrates that dimension D₄ can be the distance (e.g., height) between a base of the loader control first side first portion 762_{FS1} and the leading edge of the second protrusion 808b. Dimension D₄ can be, for example, from about 0.100 inches to about 0.160 inches, or more narrowly from about 0.127 inches to about 0.131 inches, including every 0.001 inch increment within these ranges (e.g., 0.100 inches, 0.127 inches, 0.129 inches, 0.131 inches, 0.160 inches).

FIG. 20F illustrates that dimension D₅ can be the distance (e.g., height) between a base of the loader control first side first portion 762_{FS1} and the leading edge of the first protrusion 808a. Dimension D₅ can be, for example, from about 0.130 inches to about 0.190 inches, or more narrowly from about 0.162 inches to about 0.166 inches, including every 0.001 inch increment within these ranges (e.g., 0.130 inches, 0.162 inches, 0.164 inches, 0.166 inches, 0.190 inches).

FIG. 20G illustrates the arrangement of features shown.

FIG. 20H illustrates the arrangement of features shown.

FIGS. 20I-20O illustrate that the loader control 762 (e.g., the loader control first side second portion 762_{FS2}) can have the dimensions and the arrangement of features shown. The pad 796 (e.g., the pad second side 796b, the second pad 777b) is shown transparent in FIGS. 20I-20O for illustrative purposes only.

FIG. 20I illustrates the arrangement of features shown, for example, with exemplary dimension D₆.

FIG. 20I illustrates that the loader control first side second portion 762_{FS2} can have the shape shown when viewed from below. The shape shown in FIG. 20D can be, for example, a kidney bean shape. FIG. 20I illustrates that the loader control connector 762_{C} can extend from the loader control first side second portion 762_{FS2}. The loader control connector 762_{C} can be attached to or integrated with the loader control first side second portion 762_{FS2}.

FIG. 20I illustrates that dimension D₆ can be the distance (e.g., length) between the center of the two ends 832 of the loader control connector 762_{C}. Dimension D₆ can be, for example, from about 0.350 inches to about 0.650 inches, or more narrowly from about 0.498 inches to about 0.502 inches, including every 0.001 inch increment within these ranges (e.g., 0.350 inches, 0.498 inches, 0.500 inches, 0.502 inches, 0.650 inches).

FIG. 20J illustrates the arrangement of features shown, for example, with exemplary dimensions D₇ and D₈.

FIG. 20J illustrates that dimension D₇ can be the distance (e.g., height) between a base of the loader control first side second portion 762_{FS2} and the leading edge of the base of the loader control connector 762_{C}. Dimension D₇ can be, for example, from about 0.100 inches to about 0.200 inches, or more narrowly from about 0.153 inches to about 0.159 inches, including every 0.001 inch increment within these ranges (e.g., 0.100 inches, 0.153 inches, 0.156 inches, 0.159 inches, 0.200 inches).

FIG. 20J illustrates that dimension D₈ can be the diameter of the channel 829. Dimension D₈ can be, for example, from about 0.0455 inches to about 0.0465 inches, including every 0.0001 inch increment within this range (e.g., 0.0455 inches, 0.0460 inches, 0.0465 inches).

FIG. 20K illustrates the arrangement of features shown, for example, with exemplary dimension D₉ and the angle and other dimensions shown.

FIG. 20K illustrates that the ends 832 of the loader control connector 762_{C} can be hollow, for example, so that the loader control second side 762_{SS} can be attached or removably attached to the loader control connector 762_{C}. For example, the loader control second side 762_{SS} can have extensions that can be insertable into the ends 832 of the loader control connector 762_{C}. The loader control second side 762_{SS} can be attached to the loader control connector 762_{C}, for example, with a snap fit, a friction fit, with an adhesive (e.g., glue), or any combination thereof. FIGS. 20I and 20K illustrate that the ends 832 can have holes, for example, hexagon-shaped holes. The extensions on the loader control second side 762_{SS} can be round. The hexagon-shaped holes can allow for a wider diameter range of extensions (e.g., round posts) to still provide a press fit, as hexagon-shaped holes can provide some room for the extension to deform into. FIG. 20K illustrates that dimension D₉ can be the distance (e.g., depth) of the hollow portion of the ends 832 of the loader control connector 762. Dimension D₉ can be, for example, from about 0.100 inches to about 0.200 inches, or more narrowly from about 0.140 inches to about 0.160 inches, including every 0.001 inch increment within these ranges (e.g., 0.100 inches, 0.140 inches, 0.150 inches, 0.160 inches, 0.200 inches).

FIGS. 20I-20K illustrate that the portion of the loader control connector 762_{C} between the two ends 832 can be a fin that can connect the ends 832. The fin can provide stiffness to the loader control connector 762_{C}. The fin can contact the walls of the track 764. As another example, the fin may not contact the walls of the track 764.

The loader control connector 762_{C} can be removably attachable to or integrated with the loader control first side 762_{FS} (e.g., to the loader control first side second portion 762_{FS2}). For example, FIG. 20K illustrates that the loader control connector 762_{C} can be integrated with the loader control first side 762_{FS} (e.g., with the loader control first side second portion 762_{FS2}).

FIG. 20L illustrates the arrangement of features shown, for example, with exemplary dimensions D₁₁, D₁₂, and the angle and other dimensions shown.

FIG. 20L illustrates that dimension D₁₁ can be the distance (e.g., width) between the two arms 827, for example, as measured between the two arms 827 at the bottom of the draft shown in in FIG. 20L. Dimension D₁₁ can be, for example, from about 0.160 inches to about 0.260 inches, or more narrowly from about 0.208 inches to about 0.212 inches, including every 0.001 inch increment within these ranges (e.g., 0.160 inches, 0.208 inches, 0.210 inches, 0.212 inches, 0.260 inches).

FIG. 20L illustrates that dimension D₁₂ can be, for example, from about 0.060 inches to about 0.090 inches, or more narrowly from about 0.070 inches to about 0.080 inches, including every 0.001 inch increment within these ranges (e.g., 0.060 inches, 0.070 inches, 0.075 inches, 0.080 inches, 0.090 inches).

FIG. 20M illustrates the arrangement of features shown.

FIG. 20N illustrates the arrangement of features shown, for example, with exemplary dimension D₁₃.

FIG. 20N illustrates that dimension D₁₃ can be the distance (e.g., length) between the center of the channel 829 and a terminal end of the lip 812 (e.g., a proximal terminal end of the lip 812). Dimension D₁₃ can be, for example, from about 0.300 inches to about 0.700 inches, or more narrowly from about 0.750 inches to about 0.760 inches, including every 0.001 inch increment within these ranges (e.g., 0.300 inches, 0.750 inches, 0.756 inches, 0.760 inches, 0.700 inches).

FIG. 20O illustrates the arrangement of features shown.

The loader 750 can load and/or unload shuttles 14 having males stops 412 (e.g., 1, 2, 3, 4, or more male stops 412) into and out of the device 188. The loader 750 can, for example, load and/or unload shuttles 14 having radial male stops 412. The loader 750 can, for example, load and/or unload shuttles 14 having lateral male stops 412. The loader 750 can, for example, load and/or unload shuttles 14 having one or more radial male stops 412 (e.g., one or two radial male stops 412) and one or more lateral male stops 412 (e.g., one or two lateral male stops 412). For example, the loader 750 can load and/or unload shuttles 14 having any combination of the males stops 412 shown in FIGS. 11A-11G. For example, FIGS. 14A-18 illustrate that the loader 750 can load and unload shuttles 14 having two male stops 412, for example, the male stops 412 shown in FIGS. 11D and 11E. As another example, the loader 750 can load and/or unload shuttles 14 having two male stops 412, for example, the male stops 412 shown in FIGS. 11A-11C. As yet another example, FIGS. 21A-21S illustrate that the loader 750 can load and/or unload shuttles 14 having two male stops 412, for example, the male stops 412 shown in FIGS. 11F and 11G. As another example, the loader 750 can load and/or unload shuttles 14 having zero males stops 412 into and out of the device 188. In such cases, the device 188 may have zero female stops 416 or a shuttle 14 having zero male stops 412 can be loaded into a device 188 having female stops 416 (e.g., the first female stop 416a and/or the second female stop 416b).

FIGS. 21A-21D illustrate, for example, that the loader 750 can load shuttles 14 having one or more lateral male stops 412. For example, the shuttle 14 illustrated in FIGS. 21A-21S can be the shuttle 14 shown in FIGS. 11F and 11G. FIGS. 21A-21D illustrate, for example, that the loader 750 can load suture 70 that is attached to a shuttle 14 having zero, one, or more lateral male stops 412.

FIGS. 21A-21D illustrate that the loader 750 can have the arrangement of features shown when the loader control 762 is in the loader control first position (e.g., as shown in FIG. 21A), when the loader control 762 is in a loader control intermediate position between the loader control first and second positions (e.g., as shown in FIG. 21B), when the loader control 762 is in the loader control third position and the suture holder 795 is in a closed configuration (e.g., as shown in FIG. 21C), and when the loader control 762 is in the loader control third position and the suture holder 795 is in an open configuration (e.g., as shown in FIG. 21D). The closed and open configurations of the suture holder 795 can correspond to the closed and open configurations of the loader control 762, respectively. The closed configuration in FIGS. 21A-21C of the suture holder 795 can be, for example, the loader control first closed configuration or the loader control second closed configuration. For example, the suture holder 795 can have a suture holder first closed configuration, where the suture holder first closed configuration can be the loader control first closed configuration, and the suture holder 795 can have a suture holder second closed configuration, where the suture holder second closed configuration can be the loader control second closed configuration. FIG. 21B illustrates exemplary loader control first, second, and third positions as marked by position P1, position P2, and position P3, respectively. The loader control first position can correspond to a non-loaded position of the shuttle 14. The loader control second position can correspond to a fully loaded position of the shuttle 14. The loader control third position can correspond to an overloaded position of the shuttle 14. As another example, the position of the loader control 762 in FIGS. 21C and 21D can be the loader control second position such that the shuttle 14 may not have an overloaded position.

FIGS. 21A-21C illustrate that the suture 70 can extend through the suture holder 795, for example, between the first and second pads 777a, 777b or between the pad first and second sides 796a, 796b as the loader control 762 is moved along the track 964 during loading and/or during unloading. For example, FIGS. 21A-21C illustrate that the suture 70 can be clamped between the first and second pads 777a, 777b as the loader control 762 is moved along the track 964 in direction 965 from the loader control first position to the loader control third position. FIGS. 21A-21C illustrate that when the loader control 762 is in the loader control second closed configuration, the suture holder 795 can inhibit or prevent the suture 70 from sliding and/or rolling between the first and second pads 777a, 777b as the loader control 762 is moved along the track 764 in direction 965 from the loader control first position to the loader control second position. FIGS. 21A-21C illustrate that when the loader control 762 is in the loader control second closed configuration, the loader control threshold force can be reached or exceeded at the loader control second position such that the suture 70 can slide and/or roll between the first and second pads 777a, 777b as the loader control 762 is moved from the loader control second position to the loader control third position to inhibit or prevent the suture 70 from stretching, breaking, or fraying as the shuttle 14 is moved into a loaded or overloaded position into the device 188 by moving the loader control 762 from the loader control second position to the loader control third position. FIGS. 21A-21C illustrate, for example, that the length of the suture 70 between the loader control 762 and the shuttle 14 can be longer in the when the loader control 762 is beyond the loader control second position (e.g., in the loader control third position) than when the loader control 762 is between the loader control first and second positions. FIGS. 21A-21C illustrate that the length of the suture 70 between the loader control 762 and the shuttle 14 can be constant as the loader control 762 is moved from the loader control first position to the loader control second position. The length of the suture 70 between the loader control 762 and the shuttle 14 can progressively increase as the loader control 762 is moved from the loader control second position to the loader control third position, for example, as the suture 70 slips through suture holder 795.

FIG. 21A illustrates that the loader control 762 can have the loader control first closed configuration and FIGS. 21B and 21C illustrate that the loader control can have the loader control second closed configuration. FIGS. 21A-21C illustrate that before the loader control 762 is moved from the loader control first position to the loader control second or third position in direction 765, the configuration of the loader control 762 can be changed from the loader control first closed configuration to the loader control second closed configuration. The loader control 762 can be changed from the loader control first closed configuration to the loader control second closed configuration, for example, by the user pressing the loader control first side 762_{FS} toward the loader control second side 762_{SS}, by the user pressing the loader control second side 762_{SS} toward the loader control first side 762_{FS}, by squeezing the loader control 762, by moving the loader control first side first portion 762_{FS1} toward the loader control first side second portion 762_{FS2}, by moving the loader control first side second portion 762_{FS2} toward the loader control first side first portion 762_{FS1}, or any combination thereof. As another example, FIGS. 21A-21C illustrate that the loader control 762 can have the loader control second closed configuration.

FIG. 21A illustrates that the loader 750 can be packaged without the device 188 in the device space 752.

FIG. 21A illustrates that the loader 750 can have an opener 834 that can open the loader control 762 from a closed configuration, for example, by engaging with the connector when the loader control 762 is moved along the track 764. The opener 834 can open the loader control 762 from the loader control first closed configuration. The opener 834 can open the loader control 762 from the loader control second closed configuration. The opener 834 can be, for example, an extension, a tab, a protrusion, or any combination thereof. The opener 834 can be attached to or integrated with the loader control body 751. The opener 834 can extend from the loader body 751, for example, into the track 764. The opener 834 can be flexible or rigid. The opener 834 can be positioned anywhere along the length of the track 764. For example, FIG. 21A illustrates that the opener 834 can extend from a terminal end of the track 764 such that the loader control 762 can be moved (e.g., pushed and/or pulled) into the opener 834 as the loader control 762 is moved into the loader control second or third position. FIG. 21A illustrates that the loader control 762 can be moveable relative to the opener 834. For example, FIG. 21A illustrates that the opener 834 can be in a fixed position.

When the loader control 762 is moved into the opener 834, for example, in direction 765 along the track 764, the connector 800 can be moved into contact with the opener 834 in direction 765. When the loader control 762 is moved into the opener 834, the opener 834 can move (e.g., push) the male portion 802 out of engagement with the female portion 804 or vice versa. When the loader control 762 is moved into the opener 834, the opener 834 can release the male portion 802 from the female portion 804 or vice versa. For example, FIG. 21C illustrates that when the loader control 762 is moved into the opener 834, the opener 834 can move (e.g., push) the male portion 802 in direction 826. This can disengage the male portion 802 from the female portion 804, for example, by deflecting the arm 806 and/or the protrusions 808 in direction 826. Once the male portion 802 is disengaged from the female portion 804, the spring 816 can open the loader control 762, for example, when the user lets go of the loader control 762 and/or the user can open the loader control 762. The disengagement of the male portion 802 from the female portion 804 caused by the opener 834 can provide audible and/or tactile feedback that can indicate to the user that the shuttle 14 is fully loaded into the device 188 and/or that the loader control 762 has been moved to the end of the track 764 and that they can release the loader control 762. FIG. 21D illustrates that when the loader control 762 is in the loader control third position and the user releases the loader control 762, the loader control 962 can automatically open via the spring 816.

FIGS. 21A-21D illustrate, for example, that when the loader control 762 is in the loader control first closed configuration and is moved into the opener 834 in direction 765, the arm 806 and/or first protrusion 808a can be moved (e.g., pushed) by the opener 834 in direction 826 to disengage the male portion 802 from the female portion 804. Once the male portion 802 is disengaged from the female portion 804, the spring 816 can open the loader control 762 from the loader control first closed configuration to an open configuration (e.g., to the open configuration shown in FIG. 20A or 21D). In this way, the loader control 762 can be passively and/or automatically opened from a closed configuration (e.g., the loader control first closed configuration), for example, by moving the connector 800 into the opener 834. FIGS. 21C and 21D illustrate, for example, that when the male portion 802 has been disengaged from the female portion 804 via the opener 834 and the user lets go of the loader control 762 (e.g., the user is shown transparent in FIGS. 21A-21D), the loader control 762 can automatically open. For example, the spring 816 can flip open the loader control first side first portion 762_{FS1} away from the loader control first side second portion 762_{FS2}. As another example, once the user lets go of the loader control 762, the male portion 802 can remain disengaged from the female portion 804 but the loader control 962 may not flip open. In such variations, the male portion 802 can be disengaged from the female portion 804 but the user can manually open the loader control 762.

FIGS. 21A-21D illustrate, for example, that when the loader control 762 is in the loader control second closed configuration and is moved into the opener 834 in direction 765, the arm 806, the first protrusion 808a, and/or the second protrusion 808b can be moved (e.g., pushed) by the opener 834 in direction 826 to disengage the male portion 802 from the female portion 804. Once the male portion 802 is disengaged from the female portion 804, the spring 816 can open the loader control 762 from the loader control second closed configuration to an open configuration (e.g., to the open configuration shown in FIG. 20A or 21D). In this way, the loader control 762 can be passively and/or automatically opened from a closed configuration (e.g., the loader control second closed configuration), for example, by moving the connector 800 into the opener 834. FIGS. 21C and 21D illustrate, for example, that when the male portion 802 has been disengaged from the female portion 804 via the opener 834 and the user lets go of the loader control 762 (e.g., the user is shown transparent in FIGS. 21A-21D), the loader control 762 can automatically open. For example, the spring 816 can flip open the loader control first side first portion 762_{FS1} away from the loader control first side second portion 762_{FS2}. As another example, once the user lets go of the loader control 762, the male portion 802 can remain disengaged from the female portion 804 but the loader control 962 may not flip open. In such variations, the male portion 802 can be disengaged from the female portion 804 but the user can manually open the loader control 762.

FIG. 21C illustrates that when the loader control 762 is in the loader control third position, the connector 800 can be engaged with the opener 834 and the loader control first side 762_{FS} can hang over the opener 834.

FIG. 21D illustrates the loader control 762 in an open configuration in the loader control third position after having been automatically opened by the opener 834 and released by the user.

FIG. 21C illustrates that when the loader control 762 is in the loader control third position and the loader control 762 is in a closed configuration, the suture 70 can extend diagonally across the first and second pads 777a, 777b.

FIG. 21D illustrates that when the loader control 762 is in the loader control third position and the loader control 762 is in an open configuration, the suture 70 can extend diagonally across the second pad 777b.

FIG. 21D illustrates that when the loader control 762 is in an open configuration, the spring first arm 820 can contact the first pad 777a and the spring second arm 822 can contact the second pad 777b.

FIGS. 21A-21D illustrate that the user can choose to load the shuttle 14 to the shuttle second position or to the shuttle third position by moving the loader control 762 to the loader control second position or to the loader control third position. If the user chooses to load the shuttle 14 to the shuttle second position (e.g., a non-overloaded position of shuttle 14), the user can manually open the loader control 762 when the loader control 762 is in the loader control second position by releasing the male portion 802 from the female portion 804 (e.g., by moving the male portion 802 in direction 826). If the user chooses to load the shuttle 14 to the shuttle third position (e.g., an overloaded position of the shuttle 14), the opener 834 can release the male portion 802 from the female portion 804 when the loader control 762 is in the loader control third position and the loader control 762 can automatically open once the user releases the loader control 762. As another example, if the user chooses to load the shuttle 14 to a shuttle position between the shuttle second and third positions, the user can move the loader control 762 to a loader control position between the loader control second and third positions and can manually open the loader control 762 when the loader control 762 is between the loader control second and third positions by releasing the male portion 802 from the female portion 804 (e.g., by moving the male portion 802 in direction 826).

FIGS. 21A-21D illustrate that the cap 758 can be opaque.

FIG. 21E illustrates that the cap 58 can be transparent.

FIGS. 21A and 21E illustrate that when the loader control 762 is in the loader control first position, the shuttle 14 can be completely under the cap 758.

FIG. 21B illustrates that when the loader control 762 is in a loader control position between the loader control first position and the loader control second position, the shuttle 14 can be partially outside of the cap 758, for example, in and/or above the first jaw space 752a.

FIGS. 21C and 21D illustrate that when the loader control 762 is in the loader control second or third position, the shuttle 14 can be completely outside the cap 758, for example, in and/or above the first jaw space 752a.

FIGS. 21A-21E illustrate that the male stops 412 can be movable in the tracks 756 and 760. For example, FIGS. 21A-21E illustrate that the first male stop 412a (e.g., a first lateral male stop) can be movable in the track 756 and the second male stop 412b (e.g., a second lateral male stop) can be movable in the track 760 or vice versa.

FIGS. 21A-21E illustrate, for example, that as the shuttle 14 is moved along the tracks 756 and 760 during loading and/or unloading, the first male stop 412a (e.g., a first lateral male stop) can move in the track 756 and the second male stop 412b (e.g., a second lateral male stop) can move in the track 760. FIGS. 21A-21E illustrate, for example, that as the shuttle 14 is moved along the tracks 756 and 760 during loading and/or unloading, the first male stop 412a can move along the track 756 and the second male stop 412b can move along the track 760.

When a device (e.g., the device 188) is in the device space 752, the first male stop 412a can be movable from the track 756 into the jaw track that is in the first jaw space 752a (e.g., the upper jaw track 64 or the lower jaw track 66). When a device (e.g., the device 188) is in the device space 752, the second male stop 412b can be movable from the track 760 into the jaw track that is in the first jaw space 752a (e.g., the upper jaw track 64 or the lower jaw track 66). The first male stop 412a can be moved into the device 188 before the second male stop 412b or vice versa.

FIG. 21E illustrates that the loader 750 can have a deflector 836. The shuttle 14 can be movable into the deflector 836 or vice versa, for example, during loading. The shuttle 14 can be movable against the deflector 836 or vice versa, for example, during loading. The deflector 836 can contract the shuttle 14 as the shuttle 14 is moved into (e.g., loaded into) the device 188. For example, the deflector 836 can contract one or both ends of the shuttle 14 as the shuttle 14 is moved into (e.g., loaded into) the device 188. The deflector 836 can decrease a width of the shuttle 14 as the shuttle 14 is moved into (e.g., loaded into) the device 188. For example, the deflector 836 can decrease a width of one or both ends of the shuttle 14 as the shuttle 14 is moved into (e.g., loaded into) the device 188. The deflector 836 can deflect the leading male stop 412 and/or the trailing male stop 412 as the shuttle 14 is moved into (e.g., loaded into) the device 188. As another example, the deflector 836 can deflect the leading male stop 412 and/or the trailing male stop 412 toward a longitudinal axis (e.g., a center longitudinal axis) of the shuttle 14 as the shuttle 14 is moved into (e.g., loaded into) the device 188. The deflector 836 can deflect the shuttle 14 from a shuttle non-contracted configuration (also referred to as a non-contracted configuration) to a shuttle contracted configuration (also referred to as a contracted configuration), for example, during loading of the shuttle 14 into the device. The deflector 836 can deflect the first male stop 412a and/or the second male stop 412b from a non-deflected configuration to a deflected configuration, for example, during loading of the shuttle 14 into the device. When the shuttle 14 has a non-contracted configuration, the first male stop 412a and/or the second male stop 412b can have a non-deflected configuration. When the shuttle 14 has a contracted configuration, the first male stop 412a and/or the second male stop 412b can have a deflected configuration. The deflector 836 can, for example, contract one or both ends of the shuttle 14 from a non-contracted configuration to a contracted configuration as the shuttle 14 is moved into (e.g., loaded into) the device 188. For example, the deflector 836 can deflect the leading male stop 412 from a non-contracted configuration to a contracted configuration and/or the trailing male stop 412 from a non-contracted configuration to a contracted configuration as the shuttle 14 is moved into (e.g., loaded into) the device 188.

The deflector 836 can extend into the path (e.g., loading path) of the shuttle 14. The deflector 836 can obstruct the path of the shuttle 14 as the shuttle 14 is loaded into the device 188 such that when the shuttle 14 is moved against the deflector 836, the shuttle 14 can contract. For example, the deflector 836 can obstruct the path (e.g., loading path) of the leading male stop 412 and/or the trailing male stop 412 as the shuttle 14 is loaded into the device 188 such that when the shuttle 14 is loaded, the leading male stop 412 and/or the trailing male stop 412 can engage with the deflector 836 which can move (e.g., deflect) the leading male stop and/or trailing male stop 412 toward a longitudinal axis of the shuttle 14.

The deflector 836 can be, for example, a protrusion that extends into the path (e.g., loading path) of the shuttle 14. The deflector 836 can be, for example, a protrusion that narrows the path (e.g., loading path) of the shuttle 14. The shuttle 14 can be movable past the deflector 836 during loading and/or during unloading. FIG. 21E illustrates, for example, that the deflector 836 can be a ramp. The track 756 and/or the track 760 can have the deflector 836. For example, FIG. 21E illustrates that the track 760 can have the deflector 836. The deflector 836 can be, for example, a tapered surface of the track 756 and/or a tapered surface of the track 760. For example, FIG. 21E illustrates that the deflector 836 can be a tapered surface of the track 760. The deflector 836 can be, for example, a surface of the loader body and/or a surface of the cap 758. For example, FIG. 21E illustrates that the deflector 836 can be a surface of the cap 758. The deflector 836 can be anywhere along the length of the track 756 and/or the track 760. The deflector 836 can define a longitudinal end of the track 756 and/or the track 760, for example, the longitudinal end of the track 756 or the track 760 that is closest to the entrance of the jaw track that is in the first jaw space 752a (e.g., the upper jaw track 64 or the lower jaw track 66) when the device 188 is in the device space 752. For example, FIG. 21E illustrates that the deflector 836 can define a longitudinal end of the track 760. The track 756 and/or the track 760 can thereby have a tapered end. The tapered end can form a wedge that can deflect the leading male stop 412 and/or the trailing male stop 412 as the shuttle 14 is moved into (e.g., loaded into) the device 188. FIG. 21E illustrates, for example, that the track 760 can have the deflector 836 such that the deflector 836 can be a tapered surface of the track 760. The deflector 836 can be, for example, a tapered portion of the base of the track 760. FIG. 21E illustrates, for example, that the deflector 836 can be a tapered surface of the cap 758.

During loading, the leading male stop 412 can be the first male stop 412 that is moved into the device 188, and the trailing male stop 412 can be the second male stop 412 that is moved into the device 188. The deflector 836 can deflect the leading male stop 412 and/or the trailing male stop 412, for example, toward a center longitudinal axis of the shuttle 14 as the shuttle 14 is moved into the device 188 via the user moving the loader control 762 in direction 765 and/or via the user pulling directly on the suture 70.

FIG. 21E illustrates that the first and second male stops 412a, 412b can be on opposite longitudinal ends and on opposite lateral sides of the shuttle 14, for example, as shown in FIGS. 11F and 11G. For example, the first male stop 412a can be on a shuttle first longitudinal end and on a shuttle first lateral side, and the second male stop 412b can be on a shuttle second longitudinal end and on a shuttle second lateral side. The shuttle first longitudinal end can be opposite the shuttle second longitudinal end. The shuttle first lateral side can be opposite the shuttle second lateral side. FIG. 21E illustrates, for example, that the leading male stop 412 (e.g., the first male stop 412a) can ride in the track 756 and that the trailing male stop 412 (e.g., the second male stop 412b) can ride in the track 760, or vice versa. FIG. 21E illustrates, for example, that the leading male stop 412 (e.g., the first male stop 412a) can be moveable in the track 756 and that the trailing male stop 412 (e.g., the second male stop 412b) can be movable in the track 760, or vice versa. For example, FIG. 21E illustrates that the first male stop 412a can extend toward the base of the track 756 and that the second male stop 412b can extend away from the base of the track 756. FIG. 21E illustrates, for example, that the first male stop 412a can extend away from the base of the track 760 and that the second male stop 412b can extend toward the base of the track 760. In such variations, the deflector 836 can deflect the leading male stop 412 or the trailing male stop 412. For example, FIG. 21E illustrates that the deflector 836 can deflect the trailing male stop 412—which FIG. 21E illustrates can be the second male stop 412b—as the shuttle 14 is loaded into the device 188. For example, FIG. 21E illustrates that the deflector 836 can deflect the trailing male stop 412 but not the leading male stop 412 as the shuttle 14 is loaded into the device 188. As another example, the first male stop 412a can be on a shuttle first longitudinal end and on a shuttle first lateral side, and the second male stop 412b can be on a shuttle second longitudinal end and on the shuttle first lateral side such that both the first male stop 412a and the second male stop 412b can ride in the track 756 or in the track 760.

As a first example, when the shuttle 14 is in the shuttle first position (e.g., the position shown in FIG. 21E), the leading male stop 412 and/or the trailing male stop 412 can have a non-deflected configuration. For example, during loading, as the male stops 412 enter the device 188 as the shuttle 14 is moved into the device 188, the leading male stop 412 and/or the trailing male stop 412 can be moved from the non-deflected configuration to a deflected configuration via the deflector 836. FIG. 21E illustrates, for example, that the deflector 836 can deflect the trailing male stop 412 (e.g., from the non-deflected configuration to the deflected configuration) but may not deflect the leading male stop 412 as the shuttle 14 is loaded into the device 188. The male stops 412 can be closer to a center longitudinal axis of the shuttle 14 when the male stops 412 are in the deflected configuration than when in the non-deflected configuration. The defected configuration of the male stops 412 can be, for example, a contracted configuration of the male stops 412. In such variations, the male stops 412 (e.g., the first male stop 412a and/or the second male stop 412b) can have a non-deflected configuration when the shuttle 14 is in the shuttle first position (e.g., the position shown in FIG. 21E), the male stops 412 (e.g., the first male stop 412a and/or the second male stop 412b) can have the deflected configuration (e.g., a fully deflected configuration) when in a jaw track (e.g., the upper jaw track 64 or the lower jaw track 66), for example, when the shuttle 14 is in the shuttle second position, the male stops 412 (e.g., the first male stop 412a and/or the second male stop 412b) can have a less deflected configuration (e.g., a partially deflected configuration or the non-deflected configuration) when in a female stop 416, or any combination thereof. FIG. 21E illustrates, for example, that when the shuttle 14 is in the shuttle first position, the male stops 412 (e.g., the first and second male stops 412a, 412b) can have the non-deflected configuration. The non-deflected configuration of the male stops 412 can be, for example, the configuration of the male stops 412 shown in FIGS. 11F and 11G.

As a second example, when the shuttle 14 is in the shuttle first position (e.g., the position shown in FIG. 21E), the leading male stop 412 and/or the trailing male stop 412 can have a partially deflected configuration. For example, during loading, as the male stops 412 enter the device 188 as the shuttle 14 is moved into the device 188, the leading male stop 412 and/or the trailing male stop 412 can be moved from a first deflected configuration to a second deflected configuration via the deflector 836. FIG. 21E illustrates, for example, that the deflector 836 can deflect the trailing male stop 412 (e.g., from the first deflected configuration to the second deflected configuration) but may not deflect the leading male stop 412 as the shuttle 14 is loaded into the device 188. The male stops 412 can be closer to the center longitudinal axis of the shuttle 14 when the male stops 412 are in the second deflected configuration than when in the first deflected configuration. The first deflected configuration can be, for example, a partially deflected configuration and the second deflected configuration can be, for example, a more deflected (e.g., a fully deflected) configuration. In such variations, the male stops 412 (e.g., the first and second male stops 412a, 412b) can have the first deflected configuration when the shuttle 14 is in the shuttle first position (e.g., the position shown in FIG. 21E), the male stops 412 (e.g., the first male stop 412a and/or the second male stop 412b) can have the second deflected configuration when in a jaw track (e.g., the upper jaw track 64 or the lower jaw track 66), for example, when the shuttle 14 is in the shuttle second position, the male stops 412 (e.g., the first male stop 412a and/or the second male stop 412b) can have less deflected configuration than the second deflected configuration (e.g., the non-deflected configuration, the first deflected configuration) when in a female stop 416, or any combination thereof. The first and second deflected configurations of the male stops 412 can be, for example, configurations of the male stops 412 shown in FIGS. 11F and 11G when the male stops 412 are deflected toward the center longitudinal axis of the shuttle 14.

As the shuttle 14 is moved into the device 188 from the shuttle first position, FIG. 21E illustrates that the deflector 836 can deflect the trailing male stop 412 (e.g., the second male stop 412b) toward the center longitudinal axis of the shuttle 14. Deflecting the trailing male stop 412 toward the center longitudinal axis of the shuttle 14 via the deflector 836 can move the trailing male stop 412 toward the center longitudinal axis of the shuttle 14. Deflecting the trailing male stop 412 (e.g., the second male stop 412b) via the deflector 836 can inhibit or prevent the trailing male stop 412 from catching on the tip of the jaw (e.g., the tip of the lower jaw 38 or the tip of the upper jaw 30) that is in the first jaw space 752a as the trailing male stop 412 is moved into the device 188. Deflecting the trailing male stop 412 (e.g., the second male stop 412b) via the deflector 836 can inhibit or prevent the jaw (e.g., the lower jaw 38 or the upper jaw 30) that is in the first jaw space 752a from obstructing the trailing male stop 412 from being moved into the device 188 as the shuttle 14 is loaded into the device 188. Deflecting the trailing male stop 412 (e.g., the second male stop 412b) via the deflector 836 can make the trailing end of the shuttle 14 (e.g., the side of the shuttle 14 having the trailing male stop 412) small enough to fit into the jaw that is in the first jaw space 752a. For example, deflecting the trailing male stop 412 (e.g., the second male stop 412b) via the deflector 836 can reduce the width trailing end of the shuttle 14 (e.g., the transverse width of the shuttle 14 perpendicular to the center longitudinal axis of the shuttle 14) by about 0.50 mm to about 5.00 mm, or more narrowly, by about 0.50 mm to about 3.00 mm, including every 0.01 mm increment within these ranges (e.g., 0.50 mm, 1.00 mm, 2.00 mm, 3.00 mm, 5.00 mm). Deflecting the trailing male stop 412 (e.g., the second male stop 412b) via the deflector 836 can reduce the amount of force required to load the shuttle 14 into the device 188. For variations in which the leading male stop 412 and/or the trailing male stop 412 have a partially deflected configuration (e.g., the first deflected configuration) when the shuttle 14 is in the shuttle first position (e.g., the position shown in FIG. 21E), loading the shuttle 14 from a position in which one or both of the male stops 412 have a deflected configuration can reduce the force required to load the shuttle 14 into the device 188.

Once the leading male stop 412 (e.g., the first male stop 412a) is in the device 188, the leading male stop 412 may or may not become engaged with a female stop 416 that is in the jaw in the first device space 752a (e.g., the female stop 416 in upper jaw 30 or the female stop in the lower jaw 38) as the shuttle 14 is moved further into the device 188. Once the trailing male stop 412 (e.g., the second male stop 412b) is in the device 188, the trailing male stop 412 may or may not become engaged with a female stop 416 that is in the jaw in the first device space 752a (e.g., the female stop 416 in upper jaw 30 or the female stop 416 in the lower jaw 38) as the shuttle 14 is moved further into the device 188. For example, FIG. 21H illustrates that as the shuttle 14 is moved further into the device 188, the leading male stop 412 (e.g., the first male stop 412a) can become engaged with a female stop 416 that is in the jaw in the first device space 752a and the trailing male stop 412 (e.g., the second male stop 412b) may not become engaged with a female stop 416 that is in the jaw in the first device space 752a such that the trailing male stop 412 can be in or can remain in a deflected configuration when the shuttle 14 is fully loaded into the device 188 (e.g., when the shuttle 14 is in the shuttle second or third position). The leading male stop 412 can move (e.g., automatically move) from the deflected configuration to the non-deflected configuration or to a less deflected configuration when the leading male stop 412 becomes engaged with the female stop 416. For variations in which the trailing male stop 412 (e.g., the second male stop 412b) can become engaged with a female stop 416 during loading, the trailing male stop 412 can move (e.g., automatically move) from the deflected configuration to the non-deflected configuration or to a less deflected configuration when the trailing male stop 412 becomes engaged with the female stop 416. The male stops 412 can, for example, be springs that are biased to have the non-deflected configuration (e.g., the configuration shown in FIGS. 11F and 11G).

As another example, the loader 750 (e.g., the cap 758) may not have the deflector 836. For example, the track 756 may not have the deflector 836. For example, the track 760 may not have the deflector 836. The shuttle 14 can be loaded into the device 188 with or without the deflector 836. The male stops 412 can be moved into the device 188 with or without the deflector 836. The male stops 412 can be moved into the device 188 with or without deflecting the leading male stop 412 and/or the trailing male stop 412. The male stops 412 can be moved into the device 188 with or without deflecting the leading male stop 412 and/or the trailing male stop 412 with a ramp (e.g., the deflector 836). For example, the male stops 412 can be moved into the device 188 with or without deflecting the trailing male stop 412. As another example, the deflector 836 can be attached to or integrated with the jaw that is in the first jaw space 752a. As yet another example, the shape of the tip of the jaw that is in the first jaw space 752a can deflect the leading male stop 412 and/or the trailing male stop 412 as the shuttle 14 is loaded into the device 188. As still yet another example, the jaw that is in the first jaw space 752a can have a first ramp and the loader (e.g., the cap 758) can have a second ramp (e.g., the deflector 836).

FIG. 21E illustrates that the deflector 836 can face the track 756, for example, a base of the track 756.

FIGS. 21E and 21H illustrate that the ends of the track 756 can be wider than the center of the track 756. As another example, the track 756 can have a uniform width from end to end. FIG. 21E illustrates that the ends of the track 760 can be wider than the center of the track 760. As another example, the track 760 can have a uniform width from end to end.

FIG. 21H illustrates that the female stops 416 can be lateral female stops 416. For example, FIG. 21H illustrates that the first female stop 416a can be a first lateral female stop and that the second female stop 416b can be a second lateral female stop. FIG. 21H illustrates that the upper jaw 30 can have the first female stop 416a and that the lower jaw 38 can have the second female stop 416b or vice versa. The lateral female stops 416 can extend laterally away from the shuttle tracks in the jaws. The first female stop 416a can, for example, extend laterally away from the upper jaw track 64. The second female stop 416b can, for example, extend laterally away from the lower jaw track 66. The first and second female stops 416a, 416b can extend in opposite lateral directions, for example, so that the first male stop 412a can be engageable with the first female stop 416a and so that the second male stop 412b can be engageable with the second female stop 416b. For example, FIG. 21H illustrates that when the upper jaw 30 is in the first jaw space 752a and when the lower jaw 38 is in the second jaw space 752a, the first female stop 416a can be closer to the loader body 751 than the second female stop 416b. Only a portion of the upper jaw 30 and only a portion of the lower jaw 38 are shown transparent in FIG. 21H so that the female stops 416 can be seen. Only a portion of the upper jaw 30 and only a portion of the lower jaw 38 are shown transparent in FIG. 21H so that the lateral orientation of the female stops 416 can be seen.

FIG. 21F illustrates that the device 188 can be placed in the device space 752 when the loader control 762 is in the loader control first position and the shuttle 14 is in the shuttle first position. The loader control 762 can be in a loader control open configuration (e.g., the open configuration shown in FIG. 20A), the loader control first closed configuration, or the loader control second closed configuration when the device 188 is placed in the device space 752. For example, FIG. 21F illustrates that the loader control 762 can be in the loader control first closed configuration when the device 188 is placed in the device space 752.

FIG. 21F illustrates that the lower jaw 38 can have a tissue guide 837 and that the upper jaw 30 may not have a tissue guide 837 or vice versa. As another example, both jaws can have a tissue guide 837. The tissue guides 837 can be a wedge or a tapered surface that can guide tissue between the jaws, for example, as the jaws are advanced by the user against tissue that the user would like to pass suture through. The tissue guides 837 can be attached to or integrated with one of the jaws. FIGS. 21A-21F illustrate that the first jaw space 752a and the second jaw space 752b can be sized and shaped to receive the tissue guides 837 on one or both jaws.

FIG. 21G illustrates the loader 750 without the cap 758 so that the shuttle 14 can be seen while in the shuttle first position before the shuttle 14 is loaded into the device 188 or after the shuttle 14 is unloaded from the device 188. FIG. 21G illustrates that when the shuttle 14 is in the shuttle first position, the shuttle 14 can be completely outside of the device 188, for example, between the upper and lower jaws 30, 38.

FIG. 21H illustrates that the loader 750 can have a deflector 838. The shuttle 14 can be movable into the deflector 838 or vice versa, for example, during loading. The shuttle 14 can be movable against the deflector 838 or vice versa, for example, during loading. The deflector 838 can contract the shuttle 14 as the shuttle 14 is moved into (e.g., loaded into) the device 188. For example, the deflector 838 can contract one or both ends of the shuttle 14 as the shuttle 14 is moved into (e.g., loaded into) the device 188. The deflector 838 can decrease a width of the shuttle 14 as the shuttle 14 is moved into (e.g., loaded into) the device 188. For example, the deflector 838 can decrease a width of one or both ends of the shuttle 14 as the shuttle 14 is moved into (e.g., loaded into) the device 188. For example, the deflector 838 can deflect the shuttle 14 as the shuttle 14 is moved into (e.g., loaded into) the device 188. The deflector 838 can deflect the leading male stop 412 and/or the trailing male stop 412 as the shuttle 14 is moved into (e.g., loaded into) the device 188. As another example, the deflector 838 can deflect the leading male stop 412 and/or the trailing male stop 412 toward a longitudinal axis (e.g., a center longitudinal axis) of the shuttle 14 as the shuttle 14 is moved into (e.g., loaded into) the device 188. The deflector 838 can deflect the shuttle 14 from a shuttle non-contracted configuration (also referred to as a non-contracted configuration) to a shuttle contracted configuration (also referred to as a contracted configuration), for example, during loading of the shuttle 14 into the device. The deflector 838 can deflect the first male stop 412a and/or the second male stop 412b from a non-deflected configuration to a deflected configuration, for example, during loading of the shuttle 14 into the device. When the shuttle 14 has a non-contracted configuration, the first male stop 412a and/or the second male stop 412b can have a non-deflected configuration. When the shuttle 14 has a contracted configuration, the first male stop 412a and/or the second male stop 412b can have a deflected configuration. The deflector 838 can, for example, contract one or both ends of the shuttle 14 from a non-contracted configuration to a contracted configuration as the shuttle 14 is moved into (e.g., loaded into) the device 188. For example, the deflector 838 can deflect the leading male stop 412 from a non-contracted configuration to a contracted configuration and/or the trailing male stop 412 from a non-contracted configuration to a contracted configuration as the shuttle 14 is moved into (e.g., loaded into) the device 188.

The deflector 838 can extend into the path (e.g., loading path) of the shuttle 14. The deflector 838 can obstruct the path of the shuttle 14 as the shuttle 14 is loaded into the device 188 such that when the shuttle 14 is moved against the deflector 838, the shuttle 14 can contract. For example, the deflector 838 can obstruct the path (e.g., loading path) of the leading male stop 412 and/or the trailing male stop 412 as the shuttle 14 is loaded into the device 188 such that when the shuttle 14 is loaded, the leading male stop 412 and/or the trailing male stop 412 can engage with the deflector 838 which can move (e.g., deflect) the leading male stop and/or trailing male stop 412 toward a longitudinal axis of the shuttle 14.

The deflector 838 can be, for example, a protrusion that extends into the path (e.g., loading path) of the shuttle 14. The deflector 838 can be, for example, a protrusion that narrows the path (e.g., loading path) of the shuttle 14. The shuttle 14 can be movable past the deflector 838 during loading and/or during unloading. FIG. 21H illustrates, for example, that the deflector 838 can be a ramp. The track 756 and/or the track 760 can have the deflector 838. For example, FIG. 21H illustrates that the track 756 can have the deflector 838. The deflector 838 can be, for example, a tapered surface of the track 756 and/or a tapered surface of the track 760. For example, FIG. 21H illustrates that the deflector 838 can be a tapered surface of the track 756. The deflector 838 can be, for example, a surface of the loader body 751 and/or a surface of the cap 758. For example, FIG. 21H illustrates that the deflector 838 can be a surface of the loader body 751. The deflector 838 can be anywhere along the length of the track 756 and/or the track 760. The deflector 838 can define a longitudinal end of the track 756 and/or the track 760, for example, the longitudinal end of the track 756 or the track that is closest to the entrance of the jaw track that is in the first jaw space 752a (e.g., the upper jaw track 64 or the lower jaw track 66) when the device 188 is in the device space 752. For example, FIG. 21H illustrates that the deflector 838 can define a longitudinal end of the track 756. The track 756 and/or the track 760 can thereby have a tapered end. The tapered end can form a wedge that can deflect the leading male stop 412 and/or the trailing male stop as the shuttle 14 is moved into (e.g., loaded into) the device 188. FIG. 21H illustrates, for example, that the track 756 can have the deflector 838 such that the deflector 838 can be a tapered surface of the track 756. The deflector 838 can be, for example, a tapered portion of the base of the track 756. FIG. 21H illustrates, for example, that the deflector 838 can be a tapered surface of the loader body 751.

During loading, the leading male stop 412 can be the first male stop 412 that is moved into the device 188, and the trailing male stop 412 can be the second male stop 412 that is moved into the device 188. The deflector 838 can deflect the leading male stop 412 and/or the trailing male stop 412, for example, toward the center longitudinal axis of the shuttle 14 as the shuttle 14 is moved into the device 188 via the user moving the loader control 762 in direction 765 and/or via the user pulling directly on the suture 70.

FIG. 21H illustrates that the first and second male stops 412a, 412b can be on opposite longitudinal ends and on opposite lateral sides of the shuttle 14, for example, as shown in FIGS. 11F and 11G. For example, the first male stop 412a can be on a shuttle first longitudinal end and on a shuttle first lateral side, and the second male stop 412b can be on a shuttle second longitudinal end and on a shuttle second lateral side. The shuttle first longitudinal end can be opposite the shuttle second longitudinal end. The shuttle first lateral side can be opposite the shuttle second lateral side. FIG. 21H illustrates, for example, that the first male stop 412a can extend toward the base of the track 756 and that the second male stop 412b can extend away from the base of the track 756. FIG. 21H illustrates, for example, that the first male stop 412a can extend away from the base of the track 760 and that the second male stop 412b can extend toward the base of the track 760. In such variations, the deflector 838 can deflect the leading male stop 412 or the trailing male stop 412. For example, FIG. 21H illustrates that the deflector 838 can deflect the leading male stop 412—which FIG. 21H illustrates can be the first male stop 412a—as the shuttle 14 is loaded into the device 188. For example, FIG. 21H illustrates that the deflector 838 can deflect the leading male stop 412 but not the trailing male stop 412 as the shuttle 14 is loaded into the device 188.

As a first example, as the shuttle 14 is loaded into the device 188 from the shuttle first position (e.g., the position shown in FIG. 21E), the leading male stop 412 and/or the trailing male stop 412 can be moved from the non-deflected configuration to the deflected configuration via the deflector 838. FIG. 21H illustrates, for example, that the deflector 838 can deflect the leading male stop 412 (e.g., from the non-deflected configuration to the deflected configuration) but may not deflect the trailing male stop 412 as the shuttle 14 is loaded into the device 188.

As a second example, as the shuttle 14 is loaded into the device 188 from the shuttle first position (e.g., the position shown in FIG. 21E), the leading male stop 412 and/or the trailing male stop 412 can be moved from the first deflected configuration to the second deflected configuration via the deflector 838. FIG. 21H illustrates, for example, that the deflector 838 can deflect the leading male stop 412 (e.g., from the first deflected configuration to the second deflected configuration) but may not deflect the trailing male stop 412 as the shuttle 14 is loaded into the device 188.

As the shuttle 14 is moved into the device 188 from the shuttle first position, FIG. 21H illustrates that the deflector 838 can deflect the leading male stop 412 (e.g., the first male stop 412a) toward the center longitudinal axis of the shuttle 14. Deflecting the leading male stop 412 toward the center longitudinal axis of the shuttle 14 via the deflector 838 can move the leading male stop 412 toward the center longitudinal axis of the shuttle 14. Deflecting the leading male stop 412 (e.g., the first male stop 412a) via the deflector 838 can inhibit or prevent the leading male stop 412 from catching on the tip of the jaw (e.g., the tip of the lower jaw 38 or the tip of the upper jaw 30) that is in the first jaw space 752a as the leading male stop 412 is moved into the device 188. Deflecting the leading male stop 412 (e.g., the first male stop 412a) via the deflector 838 can inhibit or prevent the jaw (e.g., the lower jaw 38 or the upper jaw 30) that is in the first jaw space 752a from obstructing the leading male stop 412 from being moved into the device 188 as the shuttle 14 is loaded into the device 188. Deflecting the leading male stop 412 (e.g., the first male stop 412a) via the deflector 838 can make the leading end of the shuttle 14 (e.g., the side of the shuttle 14 having the leading male stop 412) small enough to fit into the jaw that is in the first jaw space 752a. For example, deflecting the leading male stop 412 (e.g., the first male stop 412a) via the deflector 838 can reduce the width leading end of the shuttle 14 (e.g., the transverse width of the shuttle 14 perpendicular to the center longitudinal axis of the shuttle 14) by about 0.50 mm to about 5.00 mm, or more narrowly, by about 0.50 mm to about 3.00 mm, including every 0.01 mm increment within these ranges (e.g., 0.50 mm, 1.00 mm, 2.00 mm, 3.00 mm, 5.00 mm). Deflecting the leading male stop 412 (e.g., the first male stop 412a) via the deflector 838 can reduce the amount of force required to load the shuttle 14 into the device 188. For variations in which the leading male stop 412 and/or the leading male stop 412 have a partially deflected configuration (e.g., the first deflected configuration) when the shuttle 14 is in the shuttle first position (e.g., the position shown in FIG. 21H), loading the shuttle 14 from a position in which one or both of the male stops 412 have a deflected configuration can reduce the force required to load the shuttle 14 into the device 188.

Once the leading male stop 412 (e.g., the first male stop 412a) is in the device 188, the leading male stop 412 may or may not become engaged with a female stop 416 that is in the jaw in the first device space 752a (e.g., the female stop 416 in upper jaw 30 or the female stop 416 in the lower jaw 38) as the shuttle 14 is moved further into the device 188. For example, once the leading male stop 412 (e.g., the first male stop 412a) is in the device 188, the leading male stop 412 (e.g., the first male stop 412a) can become engaged with the female stop 416 that is in the jaw in the first device space 752a (e.g., the female stop 416 in upper jaw 30 or the female stop 416 in the lower jaw 38) as the shuttle 14 is moved further into the device 188. When the leading male stop 412 (e.g., the first male stop 412a) becomes engaged with the female stop 416, the leading male stop 412 can move (e.g., automatically move) from the deflected configuration to the non-deflected configuration or to a less deflected configuration. The male stops 412 can, for example, be springs that are biased to have the non-deflected configuration (e.g., the configuration shown in FIGS. 11F and 11G).

As another example, the loader 750 (e.g., the loader body 751) may not have the deflector 838. For example, the track 756 may not have the deflector 838. For example, the track 760 may not have the deflector 838. The shuttle 14 can be loaded into the device 188 with or without the deflector 838. The male stops 412 can be moved into the device 188 with or without the deflector 838. The male stops 412 can be moved into the device 188 with or without deflecting the leading male stop 412 and/or the trailing male stop 412. The male stops can be moved into the device 188 with or without deflecting the leading male stop 412 and/or the trailing male stop 412 with a ramp (e.g., the deflector 838). For example, the male stops 412 can be moved into the device 188 with or without deflecting the leading male stop 412. As another example, the deflector 838 can be attached to or integrated with the jaw that is in the first jaw space 752a. As yet another example, the shape of the tip of the jaw that is in the first jaw space 752a can deflect the leading male stop 412 and/or the trailing male stop 412 as the shuttle 14 is loaded into the device 188. As still yet another example, the jaw that is in the first jaw space 752a can have a first ramp and the loader (e.g., the loader body 751) can have a second ramp (e.g., the deflector 838).

FIGS. 21E and 21H illustrate that the loader 750 can have a shuttle track (e.g., the track 756 and/or the track 760). The shuttle track can have a first track and a second track. For example, the first track can be the track 756 and the second track can be the track 760, or vice versa. The shuttle 14 can be simultaneously movable in the first track (e.g., the track 756) and the second track (e.g., the track 760). The gap 786 may or may not be between the first and second tracks 756, 760 of the shuttle track. For example, FIGS. 21E and 21H illustrate that the gap 786 can be between the first and second tracks 756, 760 of the shuttle track. As another example, the shuttle track can be a single track, where the track 756 can be a first lateral side of the shuttle track and the track 760 can be a second lateral side of the shuttle track, or vice versa. The shuttle 14 can be simultaneously movable in the first and second lateral sides of the shuttle track (e.g., the tracks 756 and 760). The gap 786 may or may not be between can be between the first and second lateral sides of the shuttle track. For example, FIGS. 21E and 21H illustrate that the gap 786 can be between the first and second lateral sides of the shuttle track.

FIGS. 21E and 21H illustrate that the male stops 412 can be deflectable toward a longitudinal axis (e.g., center longitudinal axis) of the shuttle track (e.g., the track 756 and/or the track 760) by the deflector 836 and/or by the deflector 838. FIG. 21E illustrates, for example, that the trailing male stop 412 (e.g., the second male stop 412b) can be deflectable toward a longitudinal axis of the shuttle track via the deflector 836. FIG. 21H illustrates, for example, that the leading male stop 412 (e.g., the first male stop 412a) can be deflectable toward a longitudinal axis of the shuttle track via the deflector 838.

FIGS. 21E and 21H illustrate that when the male stops 412 can be deflectable toward an opposite lateral side the shuttle track (e.g., the track 756 and/or the track 760) by the deflector 836 and/or by the deflector 838. FIG. 21E illustrates, for example, that the trailing male stop 412 (e.g., the second male stop 412b) can be deflectable toward the track 756 via the deflector 836. FIG. 21E illustrates, for example, that the trailing male stop 412 (e.g., the second male stop 412b) can be closer to the track 756 when the trailing male stop 412 is in a deflected configuration than when in a non-deflected configuration. FIG. 21H illustrates, for example, that the leading male stop 412 (e.g., the first male stop 412a) can be deflectable toward the track 760 via the deflector 838. FIG. 21H illustrates, for example, that the leading male stop 412 (e.g., the first male stop 412a) can be closer to the track 760 when the leading male stop 412 is in a deflected configuration than when in a non-deflected configuration.

FIGS. 21E and 21H illustrate that when the shuttle 14 is in the shuttle first position (e.g., the position shown in FIGS. 21E and 21H), the leading male stop 412 and/or the trailing male stop 412 can have a non-deflected configuration, a partially deflected configuration, or a fully deflected configuration. For variations in which one or both of the male stops 412 have a fully deflected configuration when the shuttle 14 is in the shuttle first position, the male stops 412 may not be deflected as the male stops are loaded into (e.g., initially enter) the device 188 but can deflect outward, away from the center longitudinal axis of the shuttle 14, as the male stops 412 become engaged with a female stop 416 in the jaw in the first jaw space 752a.

The loader 750 can have the deflector 836 and/or the deflector 838. For example, the loader 750 can have the deflector 836 but not the deflector 838 such that the track 760 can have the deflector 836 and such that the track 756 may not have a ramp (e.g., the deflector 838), or vice versa. As another example, the loader 750 can have the deflector 838 but not the deflector 836 such that the track 756 can have the deflector 838 and such that the track 760 may not have a ramp (e.g., the deflector 836), or vice versa. As yet another example, the loader 750 can have the deflector 836 and the deflector 838. As still yet another example, the loader 750 may not have the deflector 836 and/or may not have the deflector 838. For variations in which the loader 750 does not have the deflector 836, the terminal edge of the track 756 and/or the jaw in the in first jaw space 752a can deflect the leading male stop 412 and/or the trailing male stop 412 as the shuttle 14 is loaded into the device 188 from the shuttle first position (e.g., the position shown in FIGS. 21E and 21H). For variations in which the loader 750 does not have the deflector 838, the terminal edge of the track 760 and/or the jaw in the in first jaw space 752a can deflect the leading male stop 412 and/or the trailing male stop 412 as the shuttle 14 is loaded into the device 188 from the shuttle first position (e.g., the position shown in FIGS. 21E and 21H).

FIG. 21H illustrates that the deflector 838 can face the track 760, for example, a base of the track 760.

FIGS. 21E and 21H illustrate the deflector 836 and the deflector 838 can be opposite each other. For example, FIGS. 21E and 21H illustrate that the deflector 836 can be in the track 760 and that the deflector 838 can be in the track 756. As another example, FIGS. 21E and 21H illustrate that the deflector 836 can be on a first side of the shuttle track (e.g., in the track 760) and that the deflector 838 can be on a second side of the shuttle track (e.g., in the track 756).

FIGS. 21I-21L illustrate that the loader 750 can have the deflector 836. FIGS. 21I-21L illustrate that the trailing male stop 412 (e.g., the second male stop 412b) can be deflected (e.g., squeezed) by the deflector 836 as the shuttle 14 is moved into the jaw that is in the first jaw stop 752a (e.g., the upper jaw 30). As another example, the lower jaw 38 can be in the first jaw stop 752a. The shuttle position illustrated in FIGS. 21I-21K can be, for example, a shuttle position between the shuttle first position and the shuttle second position (e.g., the shuttle second intermediate position). The shuttle position illustrated in FIG. 21L can be, for example, the shuttle second position or the shuttle third position.

FIG. 21I illustrates the loader 750 without the cap 758 so that the interference between the trailing male stop 412 and the jaw in the first jaw space 752a as the trailing male stop 412 enters the device 188 can be seen. The interference illustrated in FIG. 21I can result, for example, if the trailing male stop 412 is not deflected (e.g., by the deflector 836) as the trailing male stop 412 is moved into the device 188. FIG. 21I illustrates that the interference can be, for example, between a tip of the jaw in the first jaw space 752a and the trailing male stop 412. FIG. 21I illustrates that the interference can be, for example, between an outer surface of the jaw in the first jaw space 752a (e.g., an outer surface that faces the track 756 and/or the track 760) and the trailing male stop 412. As another example, the interference can be, for example, between a distal edge of the jaw in the first jaw space 752a (e.g., a distal edge adjacent the track 756 and/or the track 760) and the trailing male stop 412

FIG. 21J illustrates that the deflector 836 can deflect (e.g., squeeze) the trailing male stop 412 to remove the interference between the trailing male stop 412 and the jaw in the first jaw space 752a that can otherwise form as shown in FIG. 21I. FIG. 21J illustrates that the deflector 836 can deflect (e.g., squeeze) the trailing male stop 412 to prevent the interference shown in FIG. 21I between the trailing male stop 412 and the jaw in the first jaw space 752a from forming. For example, as the shuttle 14 transits into the device 188, the deflector 836 can push the trailing male stop 412 closed. FIG. 21J illustrates, for example, that the deflector 836 can deflect (e.g., squeeze) the trailing male stop 412 to remove the interference shown in FIG. 21I. FIG. 21J illustrates that the cap 758 can be transparent. FIG. 21J illustrates that the finger 758_{F} of the cap 758 can have the deflector 836. FIGS. 21I and 21J illustrate that the cap 758 can be removably attached to the loader 750. FIGS. 21I and 21J illustrate that the engager 836 can be removably attached to the loader 750, for example, by attaching the cap 758 to the loader 750, and that the engager 836 can be detached from the loader 750, for example, by detaching the cap 758 from the loader 750.

FIG. 21J illustrates that the deflector 836 can have a ramp length 836_{L} of about 2.0 mm to about 30.0 mm, or more narrowly, of about 2.0 mm to about 20.0 mm, or more narrowly still, of about 2.0 mm to about 10.0 mm, including every 0.1 mm increment within these ranges (e.g., 2.0 mm, 5.0 mm, 8.0 mm, 10.0 mm, 20.0 mm, 30.0 mm).

FIG. 21J illustrates that the deflector 836 can have a ramp height 836_{H} of about 2.0 mm to about 15.0 mm, or more narrowly, of about 2.0 mm to about 8.0 mm, or more narrowly still, of about 2.0 mm to about 5.0 mm, including every 0.1 mm increment within these ranges (e.g., 2.0 mm, 3.0 mm, 5.0 mm, 8.0 mm, 10.0 mm, 15.0 mm).

FIG. 21J illustrates that the ramp length 836_{L} (e.g., 10.0 mm) can be greater than the ramp height 836_{H} (e.g., 3.5 mm).

FIG. 21J illustrates that the deflector 836 can have a ramp angle 836_{A} of about 15 degrees to about 75 degrees, or more narrowly, of about 30 degrees to about 60 degrees relative to the base of the track 756 or relative to the base of the track 760, including every 1 degree increment within these ranges (e.g., 15 degrees, 30 degrees, 45 degrees, 60 degrees, 75 degrees).

FIG. 21J illustrates that the track 760 can have a track height 760_{H}. The track height 760_{H} can be the depth of the track 760. The track height 760_{H} can be about 2.0 mm to about 25.0 mm, or more narrowly, about 2.0 mm to about 15.0 mm, or more narrowly still, about 2.0 mm to about 8.0 mm, including every 0.1 mm increment within these ranges (e.g., 2.0 mm, 5.0 mm, 8.0 mm, 10.0 mm, 15.0 mm, 25.0 mm).

FIG. 21J illustrates that the track height 760_{H} (e.g., 8.0 mm) can be greater than the ramp height 836H (e.g., 3.5 mm).

FIG. 21J illustrates that the shuttle 14 can be wider than the gap 786.

FIGS. 21K and 21L illustrate the loader 750 without the cap 758 so that the position and movement of the deflected trailing male stop 412 into the device 188 can be more easily seen. FIG. 21K illustrates, for example, the arrangement of features shown in FIG. 21J without the cap 758. FIG. 21L illustrates, for example, that the trailing male stop 412 (e.g., the second male stop 412b) can be moved into the device 188 when the trailing male stop 412 is in a deflected configuration. FIGS. 21K and 21L illustrate, for example, that when the trailing male stop 412 (e.g., the second male stop 412b) is in a deflected configuration (e.g., a fully deflected configuration), the trailing male stop 412 can be moved into the jaw in the first jaw space 752a without interference from the jaw in the first jaw space 752a or without interference from the jaw in the first jaw space 752a that would substantially inhibit or outright prevent the trailing male stop 412 from being moved into the device 188. Substantial interference can be, for example, interference that would cause the suture 70 to slip through the suture holder 795 as the loader control 762 is pushed in direction 765 while the trailing male stop 412 and the jaw in the first jaw space 752a are interfering with each other (e.g., while the jaw in the first jaw space 752a is obstructing movement of the trailing male stop 412 into the device 188). Substantial interference can be, for example, interference that would cause the suture 70 to slip through the suture holder 795 before the shuttle 14 is in shuttle second position as the loader control 762 is pushed in direction 765. An example of substantial interference can be the interference shown in FIG. 21I.

FIG. 21M illustrates that when the shuttle 14 is in the shuttle first position, the male stops 412 (e.g., the first and second male stops 412a, 412b) can be in a non-deflected configuration (e.g., a non-squeezed configuration). For example, FIG. 21M illustrates that when the shuttle 14 is in the first position, the first male stop 412a can be in a non-deflected configuration in the track 756 and the second male stop 412b can be in a non-deflected configuration in the track 760 or vice versa.

FIG. 21M illustrates that the cap 758 can be transparent.

FIG. 21M illustrates the loader 750 without a device (e.g., the device 188) in the device space 752, for example, so that the cap 758 and the shuttle 14 can be more easily seen.

FIGS. 21N-21P illustrate the loader control 762 in the loader control first position when the loader control 762 is in a loader control open configuration (e.g., as shown in FIG. 21N), when the loader control 762 is in the loader control first closed configuration (e.g., as shown in FIG. 21O), and when the loader control 762 is in the loader control second closed configuration (e.g., as shown in FIG. 21P). The loader control open configuration in FIG. 21N can be, for example, a fully open configuration of the loader control 762. The spring first and second arms 820, 822 in FIG. 21N are both shown on the second pad 777b, for example, so that the first pad 777a can be clearly seen. However, in operation, the spring first arm 820 can be biased against the first pad 777a and the spring second arm 822 can be biased against the second pad 777b such that as the loader control 762 is closed from an open configuration (e.g., the open configuration shown in FIG. 21N), the spring first and second arms 820, 822 can resist the closure by pressing against the first and second pads 777a, 777b, respectively. In other words, in operation, the spring first arm 820 can be in contact with the first pad 777a and the spring second arm 822 can be in contact with the second pad 777b when the loader control 762 is in an open configuration (e.g., the open configuration shown in FIG. 21N). The open configuration shown in FIG. 21N can be, for example, a partially open configuration (e.g., a half open configuration). The open configuration shown in FIG. 21N can be, for example, a fully open configuration. As another example, the loader control 762 may not have a spring (e.g., the spring 816).

FIGS. 21N and 21O illustrate that the leading male stop 412 (e.g., the first male stop 412a) can be in a non-deflected configuration when the shuttle 14 is in the shuttle first position.

FIG. 21Q illustrates that the loader 750 can have a travel limiter 840. The travel limiter 840 can limit movement of the loader control 762 and/or the suture holder 795 along the track 764 in a direction opposite to direction 765. The travel limiter 840 can restrict movement of the loader control 762 and/or the suture holder 795 along the track 764 in a direction opposite to direction 765. The travel limiter 840 can be positioned anywhere along the track 764. For example, FIG. 21Q illustrates that the travel limiter 840 can be positioned in the location shown to limit movement of the loader control 762 away from the loader control third position (e.g., back toward the loader control second or first position) once the loader control 762 has been moved into the loader control third position. For example, FIG. 21Q illustrates that the travel limiter 840 can catch the loader control 762 in the loader control third position such that when the user lets go of the loader control 762 when the loader control 762 is in the loader control third position, the travel limiter 840 can inhibit or prevent the loader control 762 from migrating (e.g., passively migrating) away from the loader control third position (e.g., back toward the loader control second or first position). This can inhibit or prevent the shuttle 14 from migrating (e.g., passively migrating) away from a loaded position (e.g., from the shuttle third position). As another example, the travel limiter 840 can inhibit the user from moving the loader control 762 away from the loader control third position (e.g., back toward the loader control second or first position). Inhibiting passive migration and/or active movement of the loader control 765 in a direction opposite to direction 765 can help keep the shuttle 14 in the desired loaded position in the device 188.

The travel limiter 840 can releasably lock the loader control 762 in a position beyond the travel limiter 840. For example, FIG. 21Q illustrates that the travel limiter 840 can be proximal the loader control third position or can be proximal a center of the loader control 762 when the loader control 762 is in the loader control third position. The user can move (e.g., push and/or pull) the loader control 762 in a direction opposite direction 765 if the user would like to move the loader control 762 along the track 764 toward the loader control first position. The user can thus unlock the travel limitation that the travel limiter 840 can provide by moving the loader control 762 over the travel limiter 840 along the track 764 in a direction opposition to direction 765.

The travel limiter 840 can be attached to or integrated with the loader body 751. FIG. 21Q illustrates, for example, that the loader control limiter 840 can be a protrusion, an extension, an arm, a tab, an engager or any combination thereof. FIG. 21Q illustrates, for example, that the travel limiter 840 can extend laterally away from the wall of the track 764 (e.g., as shown in FIG. 21Q) toward a center of the loader 750. FIG. 21Q illustrates, for example, that the travel limiter can extend about 0.25 mm to about 2.00 mm, or more narrowly, about 0.25 mm to about 1.00 mm away from the track 764, including every 0.01 mm increment within these ranges (e.g., 0.25 mm, 0.75 mm, 1.00 mm, 2.00 mm). The travel limiter 840 can be rigid or flexible. The travel limiter 840 can have a tapered surface and a non-tapered surface. The travel limiter can have an entry surface and an exit surface. The entry surface can be closer to the loader control first position along the track 764 than the travel limiter exit surface. For example, FIG. 21Q illustrates that the travel limiter entry surface can be a tapered surface and that the travel limiter exit surface can be less tapered than the entry surface. For example, FIG. 21Q illustrates that the exit surface can be a non-tapered surface. FIG. 21Q illustrates, for example, that the travel limiter 840 can have a wedge shape. FIG. 21Q illustrates, for example, that the travel limiter 840 can be a ramp. FIG. 21Q illustrates that the travel limiter 840 can be shaped so that the loader control 762 and/or the suture holder 795 can be movable over the travel limiter 840 in direction 765 more easily than the loader control 762 and/or the suture holder 795 can be movable over the travel limiter 840 in a direction opposite to direction 765. FIG. 21Q illustrates that the travel limiter can have a shape (e.g., a wedge shape, a ramp shape), that can allow the loader control 762 and/or the suture holder 795 to be moved over the travel limiter 840 in direction 765 but that can inhibit or prevent the loader control 762 and/or the suture holder 795 from being moved over the travel limiter in a direction opposite to direction 765.

The loader 750 can have one or multiple travel limiters 840. For example, FIG. 21Q illustrates that the loader 750 can have one travel limiter 840 and that the travel limiter 840 can be in the position shown, proximal the loader control third position (e.g., between the loader control second and third positions), to inhibit movement of the loader control 762 away from the loader control third position (e.g., back toward the loader control second or first position). This can keep the shuttle 14 in the shuttle third position or inhibit the shuttle 14 from migrating (e.g., passively migrating) away from the shuttle third position (e.g., toward the shuttle second or first position). As another example, FIG. 21R illustrates that the loader the loader 750 can have the two travel limiters 840, for example, a first travel limiter 840a and a second travel limiter 840b. The loader 750 can have the first travel limiter 840a and/or the second travel limiter 840b. For example, FIG. 21R illustrates that the loader 750 can have the first travel limiter 840a and the second travel limiter 840b.

The first and second travel limiters 840a, 840b can be positioned anywhere along the track 764. For example, FIG. 21R illustrates that the first and second travel limiters 840a, 840b can be positioned in the locations shown.

The first travel limiter 840a can be in the position shown. For example, FIG. 21R illustrates that the first travel limiter 840a can be proximal the loader control second position (e.g., between the loader control first and second positions) or can be proximal a center of the loader control 762 when the loader control 762 is in the loader control second position. Such a position of the first travel limiter 840a can inhibit movement of the loader control 762 away from the loader second position in a direction opposite to direction 965 (e.g., back toward the loader control first position) once the loader control 762 has been moved into the loader control second position or beyond (e.g., between the loader control second and third positions). For example, FIG. 21R illustrates that the first travel limiter 840a can catch the loader control 762 when the loader control 762 is between the loader control second and third positions such that when the user lets go of the loader control 762 when the loader control 762 is between the loader control second and third positions, the first travel limiter 840a can inhibit or prevent the loader control 762 from migrating (e.g., passively migrating) away from the loader control second position (e.g., back toward the loader control first position). This can inhibit or prevent the shuttle 14 from migrating (e.g., passively migrating) away from the shuttle second position (e.g., toward the shuttle first position). The first travel limiter 840a can help keep the shuttle 14 in the shuttle second position.

The second travel limiter 840b can be in the position shown. For example, FIG. 21R illustrates that the second travel limiter 840b can be proximal the loader control third position (e.g., between the loader control second and third positions) or can be proximal a center of the loader control 762 when the loader control 762 is in the loader control third position. Such a position of the second travel limiter 840b can inhibit movement of the loader control 762 away from the loader third position in a direction opposite to direction 965 (e.g., back toward the loader control second or first position) once the loader control 762 has been moved into the loader control third position. For example, FIG. 21R illustrates that the second travel limiter 840b can catch the loader control 762 when the loader control 762 is in the loader control third position such that when the user lets go of the loader control 762 when the loader control 762 is in the loader control third position, the second travel limiter 840b can inhibit or prevent the loader control 762 from migrating (e.g., passively migrating) away from the loader control third position (e.g., back toward the loader control second or first position). This can inhibit or prevent the shuttle 14 from migrating (e.g., passively migrating) away from the shuttle third position (e.g., toward the shuttle second or first position). The second travel limiter 840b can help keep the shuttle 14 in the shuttle third position.

The first and second travel limiters 840a, 840b in FIG. 21R can have any of the features of the travel limiter 840, for example, described in relation to FIG. 21Q. As another example, the first and second travel limiters 840a, 840b in FIG. 21R can have the features shown in FIG. 21R.

As another example, the engagement of the leading male stop 412 with a female stop 416 when the shuttle 14 is in the shuttle second position can inhibit or prevent migration (e.g., passive migration) of the shuttle 14 away from a loaded position (e.g., away from the shuttle second position) when the loader control 762 is in the loader control second position.

As another example, the engagement of the leading male stop 412 with a female stop 416 when the shuttle 14 is in the shuttle third position can inhibit or prevent migration (e.g., passive migration) of the shuttle 14 away from a loaded position (e.g., away from the shuttle third position) when the loader control 762 is in the loader control third position.

As another example, the engagement of the leading male stop 412 with a female stop 416 when the shuttle 14 is in the shuttle third position can allow migration (e.g., passive migration) of the shuttle 14 from the shuttle third position to the shuttle second position but can inhibit or prevent migration (e.g., passive migration) of the shuttle 14 from the shuttle second position toward the shuttle first position.

As another example, the loader 750 may not have a travel limiter (e.g., the travel limiter 840. In such variations, the engagement of the leading male stop 412 with a female stop 416 can inhibit or prevent migration (e.g., passive migration) of the shuttle 14 away from a loaded position (e.g., away from the shuttle second or third position).

FIG. 21S illustrates that the opener 834 can be thinner than the depth of the track 764. FIG. 21S illustrates, for example, that the opener 834 can be positioned at the terminal end of the track 764 so that the male portion 802 (e.g., the arm 806, the first protrusion 808a, and/or the second protrusion 808b) can be moved (e.g., pushed and/or pulled) into the opener 834. FIGS. 21A and 21S illustrate that the leading edge of the opener 834 can be curved. FIGS. 21A and 21S illustrate that the leading edge of the opener 834 can have a crescent shape.

The loader 750 can have any combination of features shown in FIGS. 14A-21S. The loader control 762 can have any combination of features shown in FIGS. 14A-21S. FIGS. 14A-18 illustrate, for example, that the loader 750 can have the loader control 762 having the features shown in FIGS. 19A and 19B. FIGS. 21A-21S illustrate, for example, that the loader 750 can have the loader control 762 having the features shown in FIGS. 20A-20O. As another example, the loader 750 in FIGS. 14A-18 can have the loader control 762 shown in FIGS. 20A-20O, and the loader 750 in FIGS. 21A-21S can have the loader control 762 shown in FIGS. 19A-19B. FIGS. 14A-18 illustrate, for example, that the device 188 can have radial female stops 416 that the male stops 412 can be engageable with. FIGS. 21A-21S illustrate, for example, that the device 188 can have lateral female stops 416 that the male stops 412 can be engageable with. The loader 750 in FIGS. 21A-21S can have, for example, any of the features shown in FIG. 18 and any of the other features in FIGS. 14A-19B. For example, the loader 750 in FIGS. 21A-21S can have the suture holder 766, the holders 788, the suture grabber 790, the track 791, the ribs 792, and the rib engagers 794 as shown in FIG. 18, or any combination thereof.

The device 188 can be attached to the loader 750 when the jaws (e.g., the upper and lower jaws 30, 38) are in an open configuration. The open configuration can be a partially open configuration or a fully open configuration. The device 188 can be detached from the loader 750 when the shuttle 14 is in one of the jaws (e.g., the upper jaw 30 or the lower jaw 38). The device 188 can be detached from the loader 750 when the jaws (e.g., the upper and lower jaws 30, 38) are in an open configuration. During loading (e.g., as the shuttle 14 is being moved into the device 188), one or both of the jaws (e.g., the upper jaw 30 and/or the lower jaw 38) can be stationary or can be non-movable, for example, such that the shuttle 14 can be movable relative to the device 188 (e.g., relative to the upper jaw 30 and/or relative to the lower jaw 38).

The loader 750 can be used to load the shuttle 14 into jaws having more than one female stop 416, for example, two female stops 416.

Any of the devices (e.g., devices 188) disclosed, illustrated, contemplated, and/or incorporated herein can be removably attachable to the loader 750, for example, to load the shuttle 14 into the device 188, to unload the shuttle 14 from the device 188, or to both load the shuttle 14 into the device 188 and to unload the shuttle 14 from the device 188.

The loader 750 can have any combination of features shown in FIGS. 14A-21S. For example, the loader 750 can have a shuttle (e.g., the shuttle 14) and a shuttle track (e.g., the track 756 and/or the track 760). The shuttle 14 can be releasably attachable to the loader 750. The shuttle 14 can be releasably attached to the loader 750, for example, in the shuttle track. The shuttle 14 can be releasably attached to the loader 750, for example, to the cap 758. The shuttle 14 can be loaded onto the loader 750. The shuttle 14 can be loaded onto the loader 750, for example, into the cap 758. The shuttle 14 can be loaded onto the loader 750, for example, between the cap 758 and the loader body 751. The shuttle 14 can be loaded onto the loader 750, for example, in the shuttle track (e.g., in the track 756 and/or the track 760). The shuttle 14 can be loaded onto the loader 750 before or after a device (e.g., the device 188) is releasably attached to the loader 750. The shuttle 14 can be releasably attached to the loader 750, for example, before or after a device (e.g., the device 188) is releasably attached to the loader 750. The cap 758 can hold the shuttle 14 in position, for example, in the shuttle first position on the loader 750 until the user loads the shuttle 14 into the device 188, for example, using the loader control 762. A device (e.g., the device 188) can be removably attachable to the loader 750. The device 188 can be, for example, a suture device, a tissue piercer, or a suture device and a tissue piercer.

When the device 188 is removably attached to the loader 750, the shuttle 14 can be movable from outside the device 188 to inside the device 188. When the device 188 is removably attached to the loader 750, the shuttle 14 can be movable relative to the shuttle track (e.g., the track 756 and/or the track 760) from outside the device 188 to inside the device 188. When the device 188 is removably attached to the loader 750, the shuttle 14 can be movable from inside the shuttle track to outside the shuttle track.

The loader 750 can have a loader control (e.g., the loader control 762). The loader control 762 can have a loader control open configuration and a loader control closed configuration. The loader control 762 can be releasably lockable in the loader control closed configuration. When a suture (e.g., the suture 70) is in the loader control 762 and the loader control 762 is in the loader control closed configuration, the shuttle 14 can be movable into the device 188. When a suture (e.g., the suture 70) is in the loader control 762 and the loader control 762 is in the loader control closed configuration, the suture 70 can be movable into the device 188. When the suture 70 is in the loader control 762 and the loader control 762 is in the loader control closed configuration, the shuttle 14 and the suture 70 can be movable into the device 188. The loader control 762 can have a loader control open configuration, a loader control first closed configuration, and a loader control second closed configuration. The loader control 762 can be releasably lockable in the loader control first closed configuration, for example, via a connector (e.g., the connector 800). The loader control 762 can be releasably lockable in the loader control second closed configuration, for example, via a connector (e.g., the connector 800). When a suture (e.g., the suture 70) is in the loader control 762 and the loader control 762 is in the loader control second closed configuration, the shuttle 14 can be movable into the device 188. When a suture (e.g., the suture 70) is in the loader control 762 and the loader control 762 is in the loader control second closed configuration, the suture 70 can be movable into the device 188. When the suture 70 is in the loader control 762 and the loader control 762 is in the loader control second closed configuration, the shuttle 14 and the suture 70 can be movable into the device 188.

The loader control 762 can have an unlockable closed configuration. The loader control 762 can have a releasably lockable closed configuration.

The loader control closed configuration can be a releasably lockable closed configuration. The loader control closed configuration can be a lockable and unlockable configuration. The loader control 762 can be releasably locked in the loader control closed configuration, for example, by engaging the male portion 802 of the connector 800 with the female portion 804 of the connector 800, and the loader control 762 can be released or unlocked from the loader control closed configuration, for example, by disengaging the male portion 802 of the connector 800 from the female portion 804 of the connector 800.

The loader control first closed configuration can be a releasably lockable closed configuration. The loader control first closed configuration can be a lockable and unlockable configuration. The loader control 762 can be releasably locked in the loader control first closed configuration, for example, by engaging the male portion 802 (e.g., the first protrusion 808a) of the connector 800 with the female portion 804 of the connector 800, and the loader control 762 can be released or unlocked from the loader control first closed configuration, for example, by disengaging the male portion 802 (e.g., the first protrusion 808a) of the connector 800 from the female portion 804 of the connector 800.

The loader control second closed configuration can be a releasably lockable closed configuration. The loader control second closed configuration can be a lockable and unlockable configuration. The loader control 762 can be releasably locked in the loader control second closed configuration, for example, by engaging the male portion 802 (e.g., the second protrusion 808b) of the connector 800 with the female portion 804 of the connector 800, and the loader control 762 can be released or unlocked from the loader control second closed configuration, for example, by disengaging the male portion 802 (e.g., the second protrusion 808b) of the connector 800 from the female portion 804 of the connector 800.

The loader control 762 can be openable and closable. The loader control closed configuration can be openable. The loader control first closed configuration can be openable. The loader control second closed configuration can be openable.

The loader 750 can have a suture holder (e.g., the suture holder 795). The suture holder 795 can be releasably lockable, for example, in a closed configuration. The suture holder 795 can be a releasably lockable suture holder. The loader 750 can have a releasably lockable suture holder (e.g., the suture holder 795). The loader control 762 can have the suture holder 795. The loader control 762 can be the suture holder 795. The suture holder 795 can be the loader control 762.

The shuttle 14 can be movable via the loader control 762. The shuttle 14 can be movable via the suture holder 795.

The suture 70 can be movable via the loader control 762. The suture 70 can be movable via the suture holder 795.

The shuttle 14 and the suture 70 can be movable via the loader control 762. The shuttle 14 and the suture 70 can be movable via the suture holder 795.

The suture holder 795 can have a suture holder open configuration and a suture holder closed configuration. The suture holder open configuration can, for example, correspond to the loader control open configuration, and the suture holder closed configuration can, for example, correspond to the loader control closed configuration. The suture holder 795 can be releasably lockable in the suture holder closed configuration, for example, via the connector 800. The suture holder 795 can have a releasably lockable closed configuration. The suture holder closed configuration can be a releasably lockable closed configuration. When the suture 70 is in the suture holder 795 and the suture holder 795 is in the suture holder closed configuration, the shuttle 14 can be movable into the device 188. When the suture 70 is in the suture holder 795 and the suture holder 795 is in the suture holder closed configuration, the shuttle 14 and the suture 70 can be movable into the device 188.

The suture holder 795 can have suture holder open configuration, a suture holder first closed configuration, and a suture holder second closed configuration. The suture holder open configuration can, for example, correspond to the loader control open configuration, the suture holder first closed configuration can, for example, correspond to the loader control first closed configuration, and the suture holder second closed configuration can, for example, correspond to the loader control second closed configuration. The suture holder 795 can be releasably lockable in the suture holder first closed configuration, for example, via a connector (e.g., the connector 800). The suture holder 795 can be releasably lockable in the suture holder second closed configuration, for example, via a connector (e.g., the connector 800). When a suture (e.g., the suture 70) is in the suture holder 795 and the suture holder 795 is in the suture holder second closed configuration, the shuttle 14 can be movable into the device 188. When a suture (e.g., the suture 70) is in the suture holder 795 and the suture holder 795 is in the suture holder second closed configuration, the suture 70 can be movable into the device 188. When the suture 70 is in the suture holder 795 and the suture holder 795 is in the suture holder second closed configuration, the shuttle 14 and the suture 70 can be movable into the device 188.

The loader control 762 can have a clamp. The clamp can be openable and closable. The loader control 762 can be an openable and closable clamp. The suture holder 795 can have a clamp. The clamp can be openable and closable. The suture holder 795 can be an openable and closable clamp.

The loader 750 can have a suture (e.g., the suture 70).

A suture (e.g., the suture 70) can be clampable by the loader control 762. The suture 70 can be releasably clampable by the loader control 762. A suture (e.g., the suture 70) can be clampable by the suture holder 795. The suture 70 can be releasably clampable by the suture holder 795.

When the suture 70 is releasably clamped by the loader control 762, the shuttle 14 can be attached to the loader control 762. When the suture 70 is releasably clamped by the loader control 762, the shuttle 14 can be considered releasably attached to the loader control 762. When the suture 70 is releasably clamped by the loader control 762, the shuttle 14 can be releasably attached to the loader control 762 via the suture 70. When the suture 70 is releasably clamped by the loader control 762, the shuttle 14 can be attached to the loader control 762, for example, via the releasable connection between the suture 70 and the loader control 762.

When the suture 70 is releasably clamped by the suture holder 795, the shuttle 14 can be attached to the suture holder 795. When the suture 70 is releasably clamped by the suture holder 795, the shuttle 14 can be considered releasably attached to the suture holder 795. When the suture 70 is releasably clamped by the suture holder 795, the shuttle 14 can be releasably attached to the suture holder 795 via the suture 70. When the suture 70 is releasably clamped by the suture holder 795, the shuttle 14 can be attached to the suture holder 795, for example, via the releasable connection between the suture 70 and the suture holder 795.

The suture 70 can be more clamped when the loader control 762 is in the loader control second closed configuration than when in the loader control 762 is in the loader control first closed configuration.

The suture 70 can be more clamped when the suture holder 795 is in the suture holder second closed configuration than when in the suture holder 795 is in the suture holder first closed configuration.

When the suture 70 is in the loader control 762 and the loader control 762 is in the loader control first closed configuration, the suture 70 can be movable through the loader control 762. When the suture 70 is in the loader control 762 and the loader control 762 is in the loader control first closed configuration, the suture 70 can move (e.g., roll, translate, slip) through the loader control 762. When the suture 70 is in the loader control 762 and the loader control 762 is in the loader control second closed configuration, the loader control 762 can inhibit the suture 70 from moving (e.g., rolling, translating, slipping) through the loader control 762. When the suture 70 is in the loader control 762 and the loader control 762 is in the loader control first closed configuration, the shuttle 14 can be detached from the loader control 762. When the suture 70 is in the loader control 762 and the loader control 762 is in the loader control first closed configuration, the shuttle 14 can be considered detached from the loader control 762. When the suture 70 is in the loader control 762 and the loader control 762 is in the loader control second closed configuration, the shuttle 14 can be attached to the loader control 762. When the suture 70 is in the loader control 762 and the loader control 762 is in the loader control second closed configuration, the shuttle 14 can be considered attached to the loader control 762.

When the suture 70 is in the suture holder 795 and the suture holder 795 is in the suture holder first closed configuration, the suture 70 can be movable through the suture holder 795. When the suture 70 is in the suture holder 795 and the suture holder 795 is in the suture holder first closed configuration, the suture 70 can move (e.g., roll, translate, slip) through the suture holder 795. When the suture 70 is in the suture holder 795 and the suture holder 795 is in the suture holder second closed configuration, the suture holder 795 can inhibit the suture 70 from moving (e.g., rolling, translating, slipping) through the suture holder 795. When the suture 70 is in the suture holder 795 and the suture holder 795 is in the suture holder first closed configuration, the shuttle 14 can be detached from the suture holder 795. When the suture 70 is in the suture holder 795 and the suture holder 795 is in the suture holder first closed configuration, the shuttle 14 can be considered detached from the suture holder 795. When the suture 70 is in the suture holder 795 and the suture holder 795 is in the suture holder second closed configuration, the shuttle 14 can be attached to the suture holder 795. When the suture is in the suture holder 795 and the suture holder 795 is in the suture holder second closed configuration, the shuttle 14 can be considered attached to the suture holder 795.

The loader control 762 can be movable relative to the shuttle track (e.g., the track 756 and/or the track 760).

The suture holder 795 can be movable relative to the shuttle track (e.g., the track 756 and/or the track 760).

When the device 188 is removably attached to the loader 750, the loader control 762 can be movable relative to the device 188.

When the device 188 is removably attached to the loader 750, the suture holder 795 can be movable relative to the device 188.

The shuttle 14 can follow the loader control 762.

The shuttle 14 can be configured to follow the loader control 762.

The shuttle 14 can follow the suture holder 795.

The shuttle 14 can be configured to follow the suture holder 795.

The loader control 762 can have a connector (e.g., the connector 800). The connector can have a male portion (e.g., the male portion 802) and a female portion (e.g., the female portion 804). The connector 800 can be a clip.

The suture holder 795 can have a connector (e.g., the connector 800). The connector 800 can have a male portion (e.g., the male portion 802) and a female portion (e.g., the female portion 804). The connector 800 can be a clip.

Movement of the loader control 762 can be restrictable by the a travel limiter (e.g., the travel limiter 840). For example, movement of the loader control 762 can be preventable and/or inhibitable by the travel limiter 840. The loader control 762 can be movable relative to the travel limiter 840. The loader control 762 can be movable into the travel limiter 840. The travel limiter 840 can limit movement of the loader control 762. The travel limiter 840 can inhibit or prevent the loader control 762 from moving. The travel limiter 840 can be configured to limit travel of the loader control 762.

Movement of the suture holder 795 can be restrictable by the a travel limiter (e.g., the travel limiter 840). For example, movement of the suture holder 795 can be preventable and/or inhibitable by the travel limiter 840. The suture holder 795 can be movable relative to the travel limiter 840. The suture holder 795 can be movable into the travel limiter 840. The travel limiter 840 can limit movement of the suture holder 795. The travel limiter 840 can inhibit or prevent the suture holder 795 from moving. The travel limiter 840 can be configured to limit travel of the suture holder 795.

The travel limiter 840 can be a protrusion on the loader 750. The travel limiter 840 can have a wedge shape.

The loader control 762 can be movable from a loader control first position to a loader control second position. When the loader control 762 is in the loader control first position, the loader control 762 can be unrestricted by the travel limiter 840. When the loader control 762 is in the loader control second position, the loader control 762 can be restricted by the travel limiter 840.

The suture holder 795 can be movable from a suture holder first position to a suture holder second position. When the suture holder 795 is in the suture holder first position, the suture holder 795 can be unrestricted by the travel limiter 840. When the suture holder 795 is in the suture holder second position, the suture holder 795 can be restricted by the travel limiter 840.

The loader 750 can have an opener (e.g., the opener 834). The opener 834 can be a protrusion on the loader 750. The loader control 762 can be unlockable via the opener 834. The suture holder 795 can be unlockable via the opener 834.

The loader control 762 can be movable relative to the opener 834. The loader control 762 can be movable into the opener 834. The loader control 762 can be movable toward and/or away from the opener 834.

The suture holder 795 can be movable relative to the opener 834. The suture holder 795 can be movable into the opener 834. The suture holder 795 can be movable toward and/or away from the opener 834.

The shuttle 14 can be moveable from a shuttle first position to a shuttle second position. When the device 188 is removably attached to the loader 750 and the shuttle 14 is in the shuttle first position, the device 188 can have an open configuration. When the device 188 is removably attached to the loader 750 and the shuttle 14 is in the shuttle second position, the device 188 can have the open configuration. The shuttle 14 can be contractible via the loader 750.

The loader 750 can have a deflector (e.g., the deflector 836 and/or the deflector 838). The loader 750 can have the deflector 836. The loader can have the deflector 838. The loader 750 can have the deflector 836 and/or the deflector 838. The shuttle 14 can be contractible via the deflector (e.g., the deflector 836 and/or the deflector 838). The deflector (e.g., the deflector 836 and/or the deflector 838) can have a ramp.

A width of the shuttle 14 can be decreasable via the loader 750. A width of the shuttle can be decreasable via the deflector (e.g., the deflector 836 and/or the deflector 838).

The shuttle can have a shuttle first end and a shuttle second end. The shuttle first end and/or the shuttle second end can be contractible via the loader 750. The shuttle first end and/or the shuttle second end can be contractible via the deflector (e.g., the deflector 836 and/or the deflector 838). A width of the shuttle first end and/or a width of the shuttle second end can be decreasable via the loader 750. A width of the shuttle first end and/or a width of the shuttle second end can be decreasable via the deflector (e.g., the deflector 836 and/or the deflector 838).

The shuttle 14 can have a shuttle first longitudinal end and a shuttle second longitudinal end. The shuttle first longitudinal end can have the shuttle first tip 164a and the shuttle second longitudinal end can have the shuttle second tip 164b or vice versa. The shuttle first longitudinal end can have the first male stop 412a and/or the shuttle second longitudinal end can have the second male stop 412b or vice versa. The shuttle first longitudinal end and/or the shuttle second longitudinal end can be contractible via the loader 750. The shuttle first longitudinal end and/or the shuttle second longitudinal end can be contractible via the deflector (e.g., the deflector 836 and/or the deflector 838). A width of the shuttle first longitudinal end and/or a width of the shuttle second longitudinal end can be decreasable via the loader 750. A width the shuttle first longitudinal end and/or a width of the shuttle second longitudinal end can be decreasable via the deflector (e.g., the deflector 836 and/or the deflector 838).

The shuttle 14 can be movable against the deflector (e.g., the deflector 836 and/or the deflector 838). The shuttle first end and/or the shuttle second end can be movable against the deflector (e.g., the deflector 836 and/or the deflector 838).

The shuttle 14 can be moveable from a shuttle first position to a shuttle second position. The shuttle 14 can have a shuttle first end and a shuttle second end. The shuttle first end can have the shuttle first tip 164a and the shuttle second end can have the shuttle second tip 164b or vice versa. The shuttle first end can have the first male stop 412a and/or the shuttle second end can have the second male stop 412b or vice versa. The shuttle first end can be contractible. The shuttle second end can be contractible. When the shuttle 14 is in the shuttle first position, the shuttle first end and the shuttle second end can be outside the device 188. When the shuttle 14 is in the shuttle second position, the shuttle first end can be inside the device 188, the shuttle second end can be outside the device 188, and the shuttle second end can be in a contracted configuration. When the shuttle 14 is in the shuttle first position, the shuttle first end can be in a non-contracted configuration and the shuttle second end can be in a non-contracted configuration. When the shuttle 14 is in the shuttle second position, the shuttle first end can be in a contracted configuration. When the shuttle 14 is in the shuttle second position, the shuttle second end can be in contact with the ramp. When the shuttle 14 is in the shuttle first position, the ramp can be between the device 188 and the shuttle second end. When the shuttle 14 is in the shuttle second position, the ramp can be between the shuttle first end and the shuttle second end.

The shuttle 14 can be moveable from the shuttle second position to a shuttle third position. When the shuttle 14 is in the shuttle third position, the shuttle first end and the shuttle second end can be inside the device 188. When the shuttle 14 is in the shuttle third position, the shuttle second end can be in the contracted configuration. When the shuttle 14 is in the shuttle second position, the shuttle first end can be in a contracted configuration. When the shuttle 14 is in the shuttle third position, the shuttle first end can be less contracted than when the shuttle 14 is in the shuttle second position. When the shuttle is in the shuttle third position, the shuttle second end can be in the contracted configuration. When the shuttle 14 is in the shuttle second position, the shuttle first end can be in a contracted configuration. When the shuttle 14 is in the shuttle third position, the shuttle first end can be in a non-contracted configuration. When the shuttle 14 is in the shuttle third position, the shuttle second end can be in the contracted configuration.

The shuttle 14 can have a male stop (e.g., the male stop 412). The male stop 412 can be deflectable via the deflector (e.g., the deflector 836 and/or the deflector 838). The male stop 412 can be deflectable toward a center longitudinal axis of the shuttle 14 via the deflector (e.g., the deflector 836 and/or the deflector 838). The male stop 412 can be movable against the deflector (e.g., the deflector 836 and/or the deflector 838). The male stop 412 can be movable against the deflector (e.g., the deflector 836 and/or the deflector 838) via the loader control 762. The male stop 412 can be pushable or pullable into the deflector (e.g., the deflector 836 and/or the deflector 838). The male stop 412 can be pushable or pullable into the deflector (e.g., the deflector 836 and/or the deflector 838) via the loader control 762.

The loader control 762 can be movable from a loader control first position to a loader control second position. When the loader control 762 is in the loader control first position, the male stop 412 can have a non-deflected configuration. When the loader control 762 is in the loader control second position, the male stop 412 can have a deflected configuration. The male stop 412 can be more deflected when the loader control 762 is in the loader control second position than when the loader control 762 is in the loader control first position. When the loader control 762 is in the loader control first position, the deflector (e.g., the deflector 836 and/or the deflector 838) can be between the device 188 and the male stop 412. When the loader control 762 is in the loader control second position, the male stop 412 can be in contact with the deflector (e.g., the deflector 836 and/or the deflector 838).

When the device 188 is removably attached to the loader 750, the deflector (e.g., the deflector 836 and/or the deflector 838) can be outside of the device 188.

When the device 188 is removably attached to the loader 750, the deflector (e.g., the deflector 836 and/or the deflector 838) can be between a device first jaw and a device second jaw. When the device 188 is removably attached to the loader 750, the deflector (e.g., the deflector 836 and/or the deflector 838) can be closer to a device first jaw than to a device second jaw.

The shuttle 14 can have a first male stop (e.g., the first male stop 412a) and a second male stop (e.g., the second male stop 412b). The first male stop 412a and/or the second male stop 412b can be deflectable via the deflector (e.g., the deflector 836 and/or the deflector 838). The first male stop 412a and/or the second male stop 412b can be deflectable toward a center longitudinal axis of the shuttle via the deflector (e.g., the deflector 836 and/or the deflector 838). The first male stop 412a and/or the second male stop 412b can be movable against the deflector (e.g., the deflector 836 and/or the deflector 838). The first male stop 412a and/or the second male stop 412b can be movable against the deflector (e.g., the deflector 836 and/or the deflector 838) via the loader control 762. The first male stop 412 and/or the second male stop 412b can be pushable or pullable into the deflector (e.g., the deflector 836 and/or the deflector 838). The first male stop 412a and/or the second male stop 412b can be pushable or pullable into the deflector (e.g., the deflector 836 and/or the deflector 838) via the loader control 762. When the loader control 762 is in the loader control first position, the first male stop 412a can have a non-deflected configuration and the second male stop can have a non-deflected configuration. When the loader control 762 is in the loader control second position, the second male stop 412b can have a deflected configuration. When the loader control 762 is in the loader control second position, the first male stop 412a can have a deflected configuration. The first male stop 412a and the second male stop 412b can be more deflected when the loader control 762 is in the loader control second position than when the loader control 762 is in the loader control first position. When the loader control is in the loader control first position, the deflector (e.g., the deflector 836 and/or the deflector 838) can be between the device 188 and the first male stop 412a. When the loader control 762 is in the loader control first position, the deflector (e.g., the deflector 836 and/or the deflector 838) can be between the device 188 and the second male stop 412b. When the loader control is in the loader control second position, the first male stop 412a or the second male stop 412b can be in contact with the deflector (e.g., the deflector 836 and/or the deflector 838).

The shuttle 14 can have a shuttle first position and a shuttle second position. When the shuttle 14 is in the shuttle first position, the first male stop 412a and the second male stop 412b can be outside the device 188. When the shuttle 14 is in the shuttle second position, the first male stop 412a can be inside the device 188, the second male stop 412b can be outside the device 188, and the second male stop can be in a deflected configuration. When the shuttle 14 is in the shuttle first position, the first male stop 412a can be in a non-defected configuration and the second male stop 412b can be in a non-deflected configuration. When the shuttle 14 is in the shuttle second position, the first male stop 412a can be in a deflected configuration. When the shuttle 14 is in the shuttle second position, the second male stop 412b can be in contact with the deflector (e.g., the deflector 836 and/or the deflector 838). When the shuttle 14 is in the shuttle first position, the deflector (e.g., the deflector 836 and/or the deflector 838) can be between the device 188 and the second male stop 412b. When the shuttle 14 is in the shuttle second position, the deflector (e.g., the deflector 836 and/or the deflector 838) can be between the first male stop 412a and the second male stop 412b. When the shuttle 14 is in the shuttle first position, the first male stop 412a can be on a first side of the deflector (e.g., the deflector 836 and/or the deflector 838). When the shuttle 14 is in the shuttle second position, the first male stop 412a can be on a second side of the deflector (e.g., the deflector 836 and/or the deflector 838). The shuttle 14 can be moveable from the shuttle second position to a shuttle third position. When the shuttle 14 is in the shuttle third position, the first male stop 412a and the second male stop 412b can be inside the device 188. When the shuttle 14 is in the shuttle third position, the second male stop 412b can be in the deflected configuration. When the shuttle 14 is in the shuttle second position, the first male stop 412a can be in a deflected configuration. When the shuttle 14 is in the shuttle third position, the first male stop 412a can be less deflected than when the shuttle 14 is in the shuttle second position. When the shuttle 14 is in the shuttle third position, the second male stop can be in the deflected configuration. When the shuttle 14 is in the shuttle second position, the first male stop 412a can be in a deflected configuration. When the shuttle is in the shuttle third position, the first male stop 412a can be in a non-deflected configuration. When the shuttle 14 is in the shuttle third position, the second male stop 412b can be in the deflected configuration.

The first male stop 412a and the second male stop 412b can be movable in the shuttle track (e.g., the track 756 and/or the track 760). The first male stop 412a can be movable in a first lateral side of the shuttle track. The second male stop 412b can be movable in a second lateral side of the shuttle track. The first male stop 412a can be movable in the track 756 and the second male stop 412b can be movable in the track 760 or vice versa. The first lateral side of the shuttle track can be, for example, the track 756, and the second lateral side of the shuttle track can be, for example, the track 760. The first lateral side of the shuttle track can be, for example, the track 760, and the second lateral side of the shuttle track can be, for example, the track 756. The first lateral side of the shuttle track and the second lateral side of the shuttle track can be separated by a gap (e.g., the gap 786). The suture 70 can be movable in the gap 786.

When the shuttle 14 is in the shuttle first position, the first male stop 412a and the second male stop 412b can be outside the device 188, the first male stop 412a can be in the first lateral side of the shuttle track, and the second male stop 412b can be in the second lateral side of the shuttle track. When the shuttle 14 is in the shuttle second position, the first male stop 412a can be inside the device 188, the second male stop 412b can be outside the device 188, and the second male stop 412b can be in a deflected configuration. When the shuttle 14 is in the shuttle second position, the first male stop 412a can be outside the first lateral side of the shuttle track and the second male stop 412b can be in the second lateral side of the shuttle track.

When the first male stop 412a and/or the second male stop 412b are in the shuttle track (e.g., the track 756 and/or the track 760), the first male stop 412a and/or the second male stop 412b can be deflectable via the deflector (e.g., the deflector 836 and/or the deflector 838).

The shuttle track can have a first track and a second track. The first track can be, for example, the track 756, and the second track can be, for example, the track 760. The first track can be, for example, the track 760, and the second track can be, for example, the track 756. The first male stop 412a can be movable in the first track. The second male stop 412b can be movable in the second track. The first track and the second track can be separated by a gap (e.g., the gap 786). The suture 70 can be movable in the gap 786.

When the shuttle 14 is in the shuttle first position, the first male stop 412a and the second male stop 412b can be outside the device 188, the first male stop 412a can be in the first track, and the second male stop 412b can be in the second track. When the shuttle 14 is in the shuttle second position, the first male stop 412a can be inside the device 188, the second male stop 412b can be outside the device 188, and the second male stop 412b can be in a deflected configuration. When the shuttle 14 is in the shuttle first position, the first male stop 412a can have a non-deflected configuration in the first track and the second male stop 412b can have a non-deflected configuration in the second track. When the shuttle 14 is in the shuttle second position, the first male stop 412a can be outside the first track and the second male stop 412b can be in the second track. When the first male stop 412a is in the first track, the first male stop 412a can be deflectable via the deflector and/or when the second male stop 412b is in the second track, the second male stop 412b can be deflectable via the deflector.

The shuttle 14 can have a shuttle first longitudinal end and a shuttle second longitudinal end. The shuttle first longitudinal end can have the first male stop 412a and the shuttle second longitudinal end can have the second male stop 412b or vice versa.

The first male stop 412a can have a first male stop non-deflected configuration and a first male stop deflected configuration. When the first male stop 412a has the first male stop non-deflected configuration, the shuttle first longitudinal end can have a shuttle first longitudinal end first width. When the first male stop 412a has the first male stop deflected configuration, the shuttle first longitudinal end can have a shuttle first longitudinal end second width. The shuttle first longitudinal end second width can be less than the shuttle first longitudinal end first width.

The second male stop 412b can have a second male stop non-deflected configuration and a second male stop deflected configuration. When the second male stop 412b has the second male stop non-deflected configuration, the shuttle second longitudinal end can have a shuttle second longitudinal end first width. When the second male stop 412b has the second male stop deflected configuration, the shuttle second longitudinal end can have a shuttle second longitudinal end second width. The shuttle second longitudinal end second width can be less than the shuttle second longitudinal end first width.

The shuttle 14 can be connected to the loader control 762 via the suture 70. The shuttle 14 can be releasably connected to the loader control 762 via the suture 70. The shuttle 14 can be releasably connectable to the loader control 762 via the suture 70.

The loader control 762 can be movable from a loader control first position to a loader control second position. The shuttle 14 can be movable from a shuttle first position to a shuttle second position. When the loader control 762 is in the loader control first position, the shuttle 14 can be in the shuttle first position. When the loader control 762 is in the loader control second position, the shuttle 14 can be in the shuttle second position. The suture 70 can be under less tension when the loader control 762 is in the loader control first position than when the loader control 762 is in the loader control second position. The suture 70 can be under more tension when the loader control 762 is in the loader control second position than when the loader control 762 is in the loader control first position.

The suture 70 can be moveable from a suture first position to a suture second position. When the suture 70 is in the suture first position, the device 188 can be releasably attachable to the loader 750. When the device 188 is releasably attached to the loader 750, the suture 70 can be moveable from the suture first position to the suture second position. More of the suture 70 can be in the device 188 when the suture 70 is in the suture second position than when the suture 70 is in the suture first position. None of the suture 70 may be in the device 188 when the suture 70 is in the suture first position. Some of the suture 70 may be in the device 188 when the suture 70 is in the suture first position.

The suture 70 can be moveable from the suture first position to the suture second position via the loader control 762. When the loader control 762 is in the loader control first position, the suture 70 can be in the suture first position. When the loader control 762 is in the loader control second position, the suture 70 can be in the suture second position.

The shuttle 14 can be pullable or pushable into the device 188 via the loader control 762.

The suture 70 can be pullable or pushable into the device 188 via the loader control 762.

The shuttle 14 and the suture 70 can be pullable or pushable into the device 188 via the loader control 762.

The suture 70 can be under less tension when the suture 70 is in the suture first position than when the suture 70 is in the suture second position. The suture 70 can be under more tension when the suture 70 is in the suture second position than when the suture is in the suture first position.

Less of the shuttle 14 can be in the shuttle track when the shuttle 14 is in the shuttle second position than when in the shuttle first position.

When the shuttle 14 is in the shuttle first position, the device 188 can be releasably attached to the loader 750. When the shuttle 14 is in the shuttle second position, the device 188 can be detached from the loader 750. Less of the shuttle 14 can be in the shuttle track when the shuttle 14 is in the shuttle second position than when the shuttle 14 is in the shuttle first position. More of the shuttle 14 can be in the device 188 when the shuttle 14 is in the shuttle second position than when the shuttle 14 is in the shuttle first position.

When the device 188 is removably attached to the loader 750, the shuttle 14 can be movable relative to the shuttle track toward the device 188.

The loader 750 can have any combination of features disclosed herein.

The shuttle 14 can have any combination of features disclosed herein. For example, the shuttle 14 can have 0, 1, 2, or more male stops 412. For example, the shuttle 14 can have a first male stop 412a and/or a second male stop 412b. For variations in which the shuttle 14 has one male stop 412, the male stop 412 can be releasably engageable (e.g., releasably loadable) into a first female stop (e.g., the first female 416a) in the first jaw and the male stop 412 can be releasably engageable (e.g., releasably loadable) into a second female stop (e.g., the second female stop 416b) in the second jaw.

The shuttle 14 can contract as the shuttle 14 is moved into the device 188. The shuttle 14 can contract, for example, by a male stop 412 being moved from a non-deflected configuration to a deflected configuration. The shuttle 14 can contract, for example, as the shuttle 14 is moved (e.g., pulled and/or pushed) against a deflector. The shuttle 14 can expand as the shuttle 14 releasably engages with the device 188. The shuttle 14 can releasably engage with the device 188, for example, via the male stop 412 releasably engaging with a female stop 416. The shuttle 14 can expand, for example, by the male stop 412 moving (e.g., automatically moving) from a deflected configuration (e.g., from the contracted configuration) to a less deflected configuration (e.g., to a less contracted configuration) or to a non-deflected configuration. The shuttle 14 can expand, for example, as the male stop 412 moves into (e.g., becomes releasably engaged with) a female stop 416.

The loader 750 can have a loader control (e.g., the loader control 762). The loader control 762 can be openable and closable. The loader control 762 can have an open configuration. The loader control 762 can have a closed configuration. The loader control 762 can be releasably lockable in the closed configuration. The loader 750 can have a suture (e.g., the suture 70). When the suture 70 is in the loader control 762 and the loader control 762 is releasably locked in the closed configuration, the shuttle 14 can be movable into the device 188. When the suture 70 is in the loader control 762 and the loader control 762 is releasably locked in the closed configuration, the suture 70 can be movable into the device 188. When the suture 70 is in the loader control 762 and the loader control 762 is releasably locked in the closed configuration, the shuttle 14 and the suture 70 can be movable into the device 188. The loader control 762 can be releasably locked in the closed configuration, for example, by engaging the connector 800 (e.g., by engaging the male portion 802 with the female portion 804 and/or vice versa). The loader control 762 can be released from the closed configuration, for example, by disengaging the connector 800 (e.g., by disengaging the male portion 802 from the female portion 804 and/or vice versa). The loader control 762 can be unlocked from the closed configuration, for example, by unlocking the connector 800 (e.g., by disengaging the male portion 802 from the female portion 804 and/or vice versa). The open configuration of the loader control 762 can also be referred to as the loader control open configuration. The closed configuration of the loader control 762 can also be referred to as the loader control closed configuration.

The loader 750 can have a loader control (e.g., the loader control 762). The loader control 762 can be openable and closable. The loader control 762 can have an open configuration. The loader control 762 can have a first closed configuration. The loader control 762 can be releasably lockable in the first closed configuration. The loader control 762 can have a second closed configuration. The loader control 762 can be releasably lockable in the second closed configuration. The loader 750 can have a suture (e.g., the suture 70). The suture 70 can be more clamped when the loader control 762 is in the second closed configuration than when the loader control 762 is in the first closed configuration. When the suture 70 is in the loader control 762 and the loader control 762 is releasably locked in the second closed configuration, the shuttle 14 can be movable into the device 188. When the suture 70 is in the loader control 762 and the loader control 762 is releasably locked in the second closed configuration, the suture 70 can be movable into the device 188. When the suture 70 is in the loader control 762 and the loader control 762 is releasably locked in the second closed configuration, the shuttle 14 and the suture 70 can be movable into the device 188. When the suture 70 is in the loader control 762 and the loader control 762 is releasably locked in the first closed configuration, the suture 70 can be movable (e.g., slidable, rollable, and/or translatable) through the loader control 70. When the suture 70 is in the loader control 762 and the loader control 762 is releasably locked in the second closed configuration, the loader control 762 can inhibit the suture 70 from moving (e.g., sliding, rolling, and/or translating) through the loader control 762. When the suture 70 is in the loader control 762 and the loader control 762 is releasably locked in the first closed configuration, the shuttle 14 can be detached from the loader control 762. When the suture 70 is in the loader control 762 and the loader control 762 is releasably locked in the second closed configuration, the shuttle 14 can be attached to the loader control 762. The loader control 762 can be releasably locked in the first closed configuration, for example, by engaging the connector 800 (e.g., by engaging the first protrusion 808a with the female portion 804 and/or vice versa). The loader control 762 can be released from the first closed configuration, for example, by disengaging the connector 800 (e.g., by disengaging the first protrusion 808a from the female portion 804 and/or vice versa). The loader control 762 can be unlocked from the first closed configuration, for example, by disengaging the connector 800 (e.g., by disengaging the first protrusion 808a from the female portion 804 and/or vice versa). The loader control 762 can be releasably locked in the second closed configuration, for example, by engaging the connector 800 (e.g., by engaging the second protrusion 808b with the female portion 804 and/or vice versa). The loader control 762 can be released from the second closed configuration, for example, by disengaging the connector 800 (e.g., by disengaging the second protrusion 808b from the female portion 804 and/or vice versa). The loader control 762 can be unlocked from the second closed configuration, for example, by disengaging the connector 800 (e.g., by disengaging the second protrusion 808b from the female portion 804 and/or vice versa). The open configuration of the loader control 762 can also be referred to as the loader control open configuration. The first closed configuration of the loader control 762 can also be referred to as the loader control first closed configuration. The second closed configuration of the loader control 762 can also be referred to as the loader control second closed configuration.

The loader 750 can have a suture holder (e.g., the suture holder 795). The suture holder 795 can be openable and closable. The suture holder 795 can have an open configuration. The suture holder 795 can have a closed configuration. The suture holder 795 can be releasably lockable in the closed configuration. The loader 750 can have a suture (e.g., the suture 70). When the suture 70 is in the suture holder 795 and the suture holder 795 is releasably locked in the closed configuration, the shuttle 14 can be movable into the device 188. When the suture 70 is in the suture holder 795 and the suture holder 795 is releasably locked in the closed configuration, the suture 70 can be movable into the device 188. When the suture 70 is in the suture holder 795 and the suture holder 795 is releasably locked in the closed configuration, the shuttle 14 and the suture 70 can be movable into the device 188. The suture holder 795 can be releasably locked in the closed configuration, for example, by engaging the connector 800 (e.g., by engaging the male portion 802 with the female portion 804 and/or vice versa). The suture holder 795 can be released from the closed configuration, for example, by disengaging the connector 800 (e.g., by disengaging the male portion 802 from the female portion 804 and/or vice versa). The suture holder 795 can be unlocked from the closed configuration, for example, by unlocking the connector 800 (e.g., by disengaging the male portion 802 from the female portion 804 and/or vice versa). The open configuration of the suture holder 795 can also be referred to as the suture holder open configuration. The closed configuration of the suture holder 795 can also be referred to as the suture holder closed configuration. The loader 750 can have a loader control (e.g., the loader control 762). The loader control 762 can have the suture holder 795.

The loader 750 can have a suture holder (e.g., the suture holder 795). The suture holder 795 can be openable and closable. The suture holder 795 can have an open configuration. The suture holder 795 can have a first closed configuration. The suture holder can be releasably lockable in the first closed configuration. The suture holder 795 can have a second closed configuration. The suture holder 795 can be releasably lockable in the second closed configuration. The loader 750 can have a suture (e.g., the suture 70). The suture 70 can be more clamped when the suture holder 795 is in the second closed configuration than when the suture holder 795 is in the first closed configuration. When the suture 70 is in the suture holder 795 and the suture holder 795 is releasably locked in the second closed configuration, the shuttle 14 can be movable into the device 188. When the suture 70 is in the suture holder 795 and the suture holder 795 is releasably locked in the second closed configuration, the suture 70 can be movable into the device 188. When the suture 70 is in the suture holder 795 and the suture holder 795 is releasably locked in the second closed configuration, the shuttle 14 and the suture 70 can be movable into the device 188. When the suture 70 is in the suture holder 795 and the suture holder 795 is releasably locked in the first closed configuration, the suture 70 can be movable (e.g., slidable, rollable, and/or translatable) through the suture holder 70. When the suture 70 is in the suture holder 795 and the suture holder 795 is releasably locked in the second closed configuration, the suture holder 795 can inhibit the suture 70 from moving (e.g., sliding, rolling, and/or translating) through the suture holder 795. When the suture 70 is in the suture holder 795 and the suture holder 795 is releasably locked in the first closed configuration, the shuttle 14 can be detached from the suture holder 795. When the suture 70 is in the suture holder 795 and the suture holder 795 is releasably locked in the second closed configuration, the shuttle 14 can be attached to the suture holder 795. The suture holder 795 can be releasably locked in the first closed configuration, for example, by engaging the connector 800 (e.g., by engaging the first protrusion 808a with the female portion 804 and/or vice versa). The suture holder 795 can be released from the first closed configuration, for example, by disengaging the connector 800 (e.g., by disengaging the first protrusion 808a from the female portion 804 and/or vice versa). The suture holder 795 can be unlocked from the first closed configuration, for example, by disengaging the connector 800 (e.g., by disengaging the first protrusion 808a from the female portion 804 and/or vice versa). The suture holder 795 can be releasably locked in the second closed configuration, for example, by engaging the connector 800 (e.g., by engaging the second protrusion 808b with the female portion 804 and/or vice versa). The suture holder 795 can be released from the second closed configuration, for example, by disengaging the connector 800 (e.g., by disengaging the second protrusion 808b from the female portion 804 and/or vice versa). The suture holder 795 can be unlocked from the second closed configuration, for example, by disengaging the connector 800 (e.g., by disengaging the second protrusion 808b from the female portion 804 and/or vice versa). The open configuration of the suture holder 795 can also be referred to as the suture holder open configuration. The first closed configuration of the suture holder 795 can also be referred to as the suture holder first closed configuration. The second closed configuration of the suture holder 795 can also be referred to as the suture holder second closed configuration. The loader 750 can have a loader control (e.g., the loader control 762). The loader control 762 can have the suture holder 795.

The shuttle 14 can be moveable from a shuttle first position to a shuttle second position. The shuttle 14 can have a shuttle first end and a shuttle second end. The shuttle first end can be contractible and/or the shuttle second end can be contractible. When the shuttle 14 is in the shuttle first position, the shuttle first end and the shuttle second end can be outside the device 188. When the shuttle 14 is in the shuttle second position, the shuttle first end can be inside the device 188, the shuttle second end can be outside the device 188, and the shuttle second end can have a first contracted configuration. When the shuttle 14 is in the shuttle first position, the shuttle first end can have a non-contracted configuration and the shuttle second end can have a non-contracted configuration. When the shuttle 14 is in the shuttle second position, the shuttle first end can have a contracted configuration. The shuttle 14 can be moveable from the shuttle second position to a shuttle third position. When the shuttle 14 is in the shuttle third position, the shuttle first end and the shuttle second end can be inside the device 188. When the shuttle 14 is in the shuttle third position, the shuttle second end can have a second contracted configuration. The second contracted configuration can be more contracted than the first contracted configuration. When the shuttle 14 is in the shuttle second position, the shuttle first end can have a contracted configuration, and when the shuttle 14 is in the shuttle third position, the shuttle first end can be less contracted than when the shuttle 14 is in the shuttle second position. When the shuttle 14 is in the shuttle third position, the shuttle second end can have second contracted configuration. The second contracted configuration can be more contracted than the first contracted configuration.

The shuttle first end can be contractible and expandable. The shuttle first end can have a contracted configuration and an expanded configuration (also referred to as a non-contracted configuration). The shuttle first end can be biased to have the expanded configuration. When the shuttle first end is moved against a defector and/or a jaw of the device 188, the shuttle first end can change from the expanded configuration to the contracted configuration. The shuttle first end can change from the expanded configuration to the contracted configuration, for example, by a male stop (e.g., the first male stop 412a) being deflected by the deflector and/or by a jaw of the device 188. When the shuttle first end is moved into a female stop (e.g., the first female stop 416a), the shuttle first end can change from a contracted configuration (e.g., the contracted configuration) to a less contracted configuration or to the expanded configuration. The shuttle first end can expand, for example, by a male stop (e.g., the first male stop 412a) becoming less deflected or by returning to a non-deflected configuration. The deflector can be the deflector 836. The deflector can be the deflector 838. The first male stop 412a can be the leading male stop or the trailing male stop.

The shuttle second end can be contractible and expandable. The shuttle second end can have a contracted configuration and an expanded configuration (also referred to as a non-contracted configuration). The shuttle second end can be biased to have the expanded configuration. When the shuttle second end is moved against a defector and/or a jaw of the device 188, the shuttle second end can change from the expanded configuration to the contracted configuration. The shuttle second end can change from the expanded configuration to the contracted configuration, for example, by a male stop (e.g., the second male stop 412b) being deflected by the deflector and/or by a jaw of the device 188. When the shuttle second end is moved into a female stop (e.g., the second female stop 416b), the shuttle second end can change from a contracted configuration (e.g., the contracted configuration) to a less contracted configuration or to the expanded configuration. The shuttle second end can expand, for example, by a male stop (e.g., the second male stop 412b) becoming less deflected or by returning to a non-deflected configuration. The deflector can be the deflector 836. The deflector can be the deflector 838. The second male stop 412b can be the leading male stop or the trailing male stop. For example, the first male stop 412a can be the leading male stop and the second male stop 412b can be the trailing male stop or vice versa.

The loader 750 can have a deflector (e.g., the deflector 836 and/or the deflector 838). The shuttle 14 can have a first male stop (e.g., the first male stop 412a) and a second male stop (e.g., the second male stop 412b). The first male stop and/or the second male stop can be deflectable via the deflector. The loader 750 can have a loader control (e.g., the loader control 762). The loader control 762 can be movable from a loader control first position to a loader control second position. When the loader control 762 is in the loader control first position, the first male stop 412a can have a non-deflected configuration and the second male stop 412b can have a non-deflected configuration. When the loader control 762 is in the loader control second position, the second male stop can have a deflected configuration. When the loader control 762 is in the loader control second position, the first male stop 412a can have a deflected configuration. The shuttle 14 can be moveable from a shuttle first position to a shuttle second position. When the shuttle 14 is in the shuttle first position, the first male stop 412a and the second male stop 412b can be outside the device 188. When the shuttle 14 is in the shuttle second position, the first male stop 412a can be inside the device 188, the second male stop 412b can be outside the device 188, and the second male stop 412b can have a first deflected configuration. When the shuttle 14 is in the shuttle first position, the first male stop 412a can have a non-defected configuration and the second male stop 412b can have a non-deflected configuration. When the shuttle 14 is in the shuttle second position, the first male stop 412a can have a deflected configuration. The shuttle can be moveable from the shuttle second position to a shuttle third position. When the shuttle 14 is in the shuttle third position, the first male stop 412a and the second male stop 412b can be inside the suture device. When the shuttle 14 is in the shuttle third position, the second male stop 412b can have a second deflected configuration. The second deflected configuration can be more deflected than the first deflected configuration. When the shuttle 14 is in the shuttle second position, the first male stop 412a can have a deflected configuration, and when the shuttle 14 is in the shuttle third position, the first male stop 412a can be less deflected than when the shuttle 14 is in the shuttle second position. When the shuttle 14 is in the shuttle third position, the second male stop 412b can have a second deflected configuration. The second deflected configuration can be more deflected than the first deflected configuration. When the shuttle 14 is in the shuttle second position, the first male stop 412a can have a deflected configuration, and when the shuttle 14 is in the shuttle third position, the first male stop 412a can have a non-deflected configuration. When the shuttle 14 is in the shuttle third position, the second male stop 412b can have a second deflected configuration. The second deflected configuration can be more deflected than the first deflected configuration.

When the first male stop 412a has a deflected configuration, the shuttle 14 can have a contracted configuration.

When the first male stop 412a has a non-deflected configuration, the shuttle 14 can have an expanded configuration.

When the second male stop 412b has a deflected configuration, the shuttle 14 can have a contracted configuration.

When the second male stop 412b has a non-deflected configuration, the shuttle 14 can have an expanded configuration.

The loader 750 can have a suture (e.g., the suture 70) and a loader control (e.g., the loader control 762). The shuttle 14 can be connectable to the loader control 762 via the suture 70. The shuttle 14 and/or the suture 70 can be pullable and/or pushable into the device 188 via the loader control 762. When the suture device 18 is removably attached to the loader 750, the shuttle 14 can be movable relative to the shuttle track toward the device 188 via the loader control 762 and/or away from the device 188 via the loader control 762.

A method can have any combination of steps disclosed herein. FIGS. 14A-21S illustrate, for example, a method of loading a device (e.g., the device 188) using a loader (e.g., the loader 750). The method can include loading a shuttle (e.g., the shuttle 14) into the device 188.

Loading the shuttle 14 into the device 188 can include contracting the shuttle 14. Contracting the shuttle 14 can include decreasing a width of the shuttle 14. Contracting the shuttle 14 can include moving a male stop (e.g., the first male stop 412a and/or the second male stop 412b). Contracting the shuttle 14 can include deflecting a male stop (e.g., the first male stop 412a and/or the second male stop 412b). Contracting the shuttle 14 can include moving a male stop (e.g., the first male stop 412a and/or the second male stop 412b) against a deflector.

Loading the shuttle 14 into the device 188 can include expanding the shuttle 14. Expanding the shuttle 14 can include increasing a width of the shuttle 14. Expanding the shuttle 14 can include moving a male stop (e.g., the first male stop 412a and/or the second male stop 412b) into a female stop 416. Expanding the shuttle 14 can include deflecting a male stop (e.g., the first male stop 412a and/or the second male stop 412b), for example, into a female stop 416. Expanding the shuttle 14 can include a male stop (e.g., the first male stop 412a and/or the second male stop 412b) moving into a female stop (e.g., automatically moving into a female stop 416) as the shuttle 14 is moved into the device 188.

Loading the shuttle 14 can include moving the shuttle 14 from a non-loaded configuration to a loaded configuration by moving the loader control 762 from the loader control first position to the loader control second position. When the loader control 762 is in the loader control first position, the shuttle 14 can be in the non-loaded configuration. When the loader control 762 is in the loader control second position, the shuttle 14 can be in the loaded configuration.

Loading the shuttle 14 can include contracting a leading end (e.g., a first end) of the shuttle 14. The leading end of the shuttle 14 can contract, for example, by a leading male stop (e.g., the first male stop 412a) moving toward an opposite side of the shuttle 14 and/or toward a center longitudinal axis of the shuttle 14 as the shuttle 14 enters the device 188 and/or as the shuttle 14 is moved against a deflector. The leading end of the shuttle 14 can contract, for example, by the leading male stop (e.g., the first male stop 412a) moving toward the side of the shuttle 14 that a trailing male stop (e.g., the second male stop 412b) can be on. The leading male stop can be on a first lateral side of the shuttle 14 and the trailing male stop can be on a second lateral side of the shuttle 14. The first lateral side of the shuttle 14 can be opposite the second lateral side of the shuttle 14.

Loading the shuttle 14 can include contracting and expanding a leading end (e.g., a first end) of the shuttle 14. The leading end of the shuttle 14 can contract, for example, by a leading male stop (e.g., the first male stop 412a) moving toward an opposite side of the shuttle 14 and/or toward a center longitudinal axis of the shuttle 14 as the shuttle 14 enters the device 188 and/or as the shuttle 14 is moved against a deflector. The leading end of the shuttle 14 can expand, for example, by the leading male stop moving away from the opposite side of the shuttle 14 and/or away from the center longitudinal axis of the shuttle 14 as the leading male stop enters a female stop (e.g., the female stop 416) in the upper jaw or the lower jaw of the device 188.

Loading the shuttle 14 can include contracting a trailing end (e.g., a second end) of the shuttle 14. The trailing end of the shuttle 14 can contract, for example, by a trailing male stop (e.g., the second male stop 412b) moving toward an opposite side of the shuttle 14 and/or toward a center longitudinal axis of the shuttle 14 as the shuttle 14 enters the device 188 and/or as the shuttle 14 is moved against a deflector. The trailing end of the shuttle 14 can contract, for example, by the trailing male stop (e.g., the second male stop 412b) moving toward the side of the shuttle 14 that the leading male stop (e.g., the first male stop 412a) can be on.

Loading the shuttle 14 can include contracting and expanding a trailing end (e.g., a second end) of the shuttle 14. The trailing end of the shuttle 14 can contract, for example, by a trailing male stop (e.g., the second male stop 412b) moving toward an opposite side of the shuttle 14 and/or toward a center longitudinal axis of the shuttle 14 as the shuttle 14 enters the device 188 and/or as the shuttle 14 is moved against a deflector. The trailing end of the shuttle 14 can expand, for example, by the trailing male stop moving away from the opposite side of the shuttle 14 and/or away from the center longitudinal axis of the shuttle 14 as the trailing male stop enters a female stop (e.g., the female stop 416) in the upper jaw or the lower jaw of the device 188.

Loading the shuttle 14 can include moving the shuttle 14 from a non-loaded configuration to a loaded configuration by moving the loader control 762 from the loader control first position to the loader control second position. When the loader control 762 is in the loader control first position, the shuttle 14 can be in the non-loaded configuration. When the loader control 762 is in the loader control second position, the shuttle 14 can be in the loaded configuration.

When the shuttle 14 is in the non-loaded configuration, the leading end of the shuttle 14 can have a non-contracted configuration and the trailing end of the shuttle 14 can have a non-contracted configuration. When the shuttle 14 is in the non-loaded configuration, the leading end of the shuttle 14 can have a non-contracted configuration or a partially contracted configuration and the trailing end of the shuttle 14 can have a non-contracted configuration or a partially contracted configuration. When the shuttle 14 is in the non-loaded configuration, for example, the leading male stop can have a non-deflected configuration and the trailing male stop can have a non-deflected configuration.

When the shuttle 14 is in the loaded configuration, the leading end of the shuttle 14 can have a non-contracted or a partially contracted configuration and the trailing end of the shuttle 14 can have a partially contracted configuration or a fully contracted configuration. When the shuttle 14 is in the loaded configuration, for example, the leading male stop can have a non-deflected configuration or a partially deflected configuration and the trailing male stop can have a partially deflected configuration or a fully deflected configuration.

When the shuttle 14 is in the loaded configuration, the trailing end of the shuttle 14 can have a non-contracted or a partially contracted configuration and the leading end of the shuttle 14 can have a partially contracted configuration or a fully contracted configuration. When the shuttle 14 is in the loaded configuration, for example, the trailing male stop can have a non-deflected configuration or a partially deflected configuration and the leading male stop can have a partially deflected configuration or a fully deflected configuration.

The method can include loading a suture (e.g., the suture 70) into the device 188. Loading the suture 70 into the device 188 can include moving the suture 70 from a non-loaded configuration to a loaded configuration by moving the loader control 762 from the loader control first position to the loader control second position. When the loader control 762 is in the loader control first position, the suture 70 can be in the non-loaded configuration. When the loader control 762 is in the loader control second position, the suture 70 can be in the loaded configuration.

Loading the shuttle 14 and the suture 70 can include moving the shuttle 14 and the suture 70 from a non-loaded configuration to a loaded configuration by moving the loader control 762 from the loader control first position to the loader control second position. When the loader control 762 is in the loader control first position, the shuttle 14 and the suture 70 can be in the non-loaded configuration. When the loader control 762 is in the loader control second position, the shuttle 14 and the suture 70 can be in the loaded configuration.

The method can include removably attaching the device 188 to the loader 750.

The method can include detaching the device 188 from the loader 750.

A method can have any combination of steps disclosed herein. FIGS. 14A-21S illustrate, for example, a method of loading and/or unloading a device (e.g., the device 188). The method can include loading a shuttle (e.g., the shuttle 14) into the device 188. The method can include loading a shuttle (e.g., the shuttle 14) into the device 188, for example, from a loader (e.g., the loader 750). Loading the shuttle 14 can include contracting and/or expanding the shuttle 14. During loading, the shuttle 14 can contract, for example, when a male stop (e.g., the male stop 412) engages with a deflector (e.g., the deflector 836 or the deflector 838) or with a jaw of the device 188. During loading, the shuttle 14 can expand, for example, when the male stop 412 engages with a female stop (e.g., the female stop 416). The method can include unloading the shuttle 14 from the device 188, for example, into the loader 750. Unloading the shuttle 14 can include contracting and/or expanding the shuttle 14. During unloading, the shuttle 14 can contract, for example, when a male stop (e.g., the male stop 412) engages with a lip of a female stop (e.g., the female stop 416) as the male stop 412 exits the female stop 416. During unloading, the shuttle 14 can expand, for example, when the male stop 412 enters the shuttle track in the loader 750. The device 188 can be, for example, a tissue piercer. The device 188 can be, for example, a suture device. The device 188 can be, for example, a tissue piercer and a suture device. The device 188 can be for example, a suture device that can pierce tissue, for example, with the shuttle 14. The device 188 can be for example, a suture device that can pass suture through tissue, for example, with the shuttle 14.

The loader 750 can have any combination of features shown in FIGS. 14A-21S. For example, the loader 750 can have a body (e.g., the body 751) having a device space (e.g., the device space 752), a loader control (e.g., the loader control 762), and a shuttle (e.g., the shuttle 14). The shuttle 14 can be moveable from a shuttle first position to a shuttle second position via the loader control 762. A device (e.g., the device 188) can be positionable in the device space 752. The device 188 can be, for example, a tissue piercer. The device 188 can be, for example, a suture device. The device 188 can be, for example, a tissue piercer and a suture device. The device 188 can be for example, a suture device that can pierce tissue, for example, with the shuttle 14. The device 188 can be for example, a suture device that can pass suture through tissue, for example, with the shuttle 14. When the device 188 is positioned in the device space 752, the shuttle 14 can be moveable from the shuttle first position to the shuttle second position. The shuttle 14 can be more contracted when the shuttle 14 is in the shuttle second position than when the shuttle 14 is in the shuttle first position.

When the shuttle 14 is in the shuttle first position, the shuttle 14 can have a non-contracted configuration, and when the shuttle 14 is in the shuttle second position, the shuttle can have a contracted configuration. The shuttle 14 can be wider when the shuttle 14 is in the non-contracted configuration than when the shuttle 14 is in the contracted configuration. When the shuttle 14 is in the shuttle first position, the shuttle 14 can be outside the device 188, and when the shuttle 14 is in the shuttle second position, the shuttle 14 can be inside the device 188. A first end of the shuttle 14 can be wider when the shuttle 14 is in the non-contracted configuration than when the shuttle 14 is in the contracted configuration, and/or a second end of the shuttle 14 can be wider when the shuttle 14 is in the non-contracted configuration than when the shuttle 14 is in the contracted configuration. When the shuttle 14 is in the shuttle first position, the first end of the shuttle 14 and the second end of the shuttle 14 can be outside the device 188, and when the shuttle 14 is in the shuttle second position, the first end of the shuttle 14 can be inside the device 188 and the second end of the shuttle 14 can be outside the device 188. When the shuttle 14 is in the shuttle first position, the first end of the shuttle 14 and the second end of the shuttle 14 can be outside the device 188, and when the shuttle 14 is in the shuttle second position, the first end of the shuttle 14 and the second end of the shuttle 14 can be inside the device 188. When the shuttle 14 has the non-contracted configuration, a first longitudinal end of the shuttle 14 can have a non-contracted configuration and a second longitudinal end of the shuttle 14 can have a non-contracted configuration. When the shuttle 14 has the contracted configuration, a first longitudinal end of the shuttle 14 can have a contracted configuration and a second longitudinal end of the shuttle 14 can have a contracted configuration, and/or when the shuttle 14 has the contracted configuration, the first longitudinal end of the shuttle 14 can be more contracted than the second longitudinal end of the shuttle 14. When the shuttle 14 has the contracted configuration, the first longitudinal end of the shuttle 14 can be inside the device 188 and the second longitudinal end of the shuttle 14 can be outside the device 188. When the shuttle 14 has the contracted configuration, the first longitudinal end of the shuttle 14 and the second longitudinal end of the shuttle 14 can be inside the device 188. When the shuttle 14 has the non-contracted configuration, a width of a contractible and expandable portion of the first longitudinal end of the shuttle 14 can the same as a width of a contractible and expandable portion of the second longitudinal end of the shuttle 14.

The contractible and expandable portion of the first longitudinal end of the shuttle 14 can be, for example, the portion of the first longitudinal end of the shuttle 14 having the first male stop 412a. The contractible and expandable portion of the first longitudinal end of the shuttle 14 can be, for example, the first male stop 412a. The width of the contractible and expandable portion of the first longitudinal end of the shuttle 14, can be, for example, the width of the shuttle 14 between a first lateral terminal end of the shuttle 14 and a second lateral terminal end of the shuttle 14, where the first lateral terminal end of the shuttle 14 on the first longitudinal end of the shuttle 14 can be, for example, an edge or surface of the first male stop 412a, and where the second lateral terminal end of the shuttle 14 on the first longitudinal end of the shuttle 14 can be, for example, an edge or surface of the shuttle 14 opposite the first male stop 412a.

The contractible and expandable portion of the second longitudinal end of the shuttle can be, for example, the portion of the second longitudinal end of the shuttle 14 having the second male stop 412b. The contractible and expandable portion of the second longitudinal end of the shuttle 14 can be, for example, the second male stop 412b. The width of the contractible and expandable portion of the second longitudinal end of the shuttle 14, can be, for example, the width of the shuttle 14 between a first lateral terminal end of the shuttle 14 and a second lateral terminal end of the shuttle 14, where the first lateral terminal end of the shuttle 14 on the second longitudinal end of the shuttle 14 can be, for example, an edge or surface of the second male stop 412b, and where the second lateral terminal end of the shuttle 14 on the second longitudinal end of the shuttle 14 can be, for example, an edge or surface of the shuttle 14 opposite the first male stop 412a.

When the shuttle 14 has the non-contracted configuration, the width of the contractible and expandable portion of the first longitudinal end of the shuttle 14 and the width of the contractible and expandable portion of the second longitudinal end of the shuttle 14 can be measured along an axis perpendicular to a center longitudinal axis of the shuttle.

When the shuttle 14 has the non-contracted configuration, a width of the widest portion of a contractible and expandable section of the first longitudinal end of the shuttle 14 can be less than or greater than a width of the widest portion of a contractible and expandable section of the second longitudinal end of the shuttle 14.

When the shuttle 14 has the contracted configuration, a width of the widest portion of a contractible and expandable section of the first longitudinal end of the shuttle 14 can be the same as a width of the widest portion of a contractible and expandable section of the second longitudinal end of the shuttle 14.

When the shuttle 14 has the contracted configuration, a width of the first longitudinal end of the shuttle 14 can be less than a width of the second longitudinal end of the shuttle 14, or when the shuttle 14 has the contracted configuration, a width of the widest portion of a contractible and expandable section of the first longitudinal end of the shuttle 14 can be less than a width of the widest portion of a contractible and expandable section of the second longitudinal end of the shuttle 14.

When the shuttle 14 has the non-contracted configuration, the width of the first longitudinal end of the shuttle 14 and the width of the second longitudinal end of the shuttle 14 can be measured along an axis perpendicular to a center longitudinal axis of the shuttle.

The loader 750 can have a deflector (e.g., the deflector 836 and/or the deflector 838). The shuttle 14 can be engageable with the deflector. When the shuttle 14 is engaged with the deflector, the shuttle 14 can have the contracted configuration.

The loader 750 can have a first deflector (e.g., the deflector 836) and a second deflector (e.g., the deflector 838). The shuttle 14 can be engageable with the first deflector and/or with the second deflector. When the shuttle 14 is engaged with the first deflector and/or with the second deflector, the shuttle 14 can have the contracted configuration.

The loader 750 can have a suture (e.g., the suture 70). The shuttle 14 can be connectable to the loader control 762 via the suture 70. The suture 70 can be under more tension when the shuttle 14 is in the shuttle second position than when the shuttle 14 is in the shuttle first position.

The loader 750 can have any combination of features shown in FIGS. 14A-21S. For example, the loader 750 can have a body (e.g., the body 751) having a device space (e.g., the device space 752), a loader control (e.g., the loader control 762), a deflector (e.g., the deflector 836 and/or the deflector 838), and a shuttle (e.g., the shuttle 14). The shuttle 14 can be contractible and expandable. The shuttle 14 can be contractible via the deflector. When the shuttle 14 is in contact with the deflector, the shuttle 14 can have a contracted configuration. The shuttle 14 can be biased to have an expanded configuration. The deflector can contract the shuttle 14, for example, from the expanded configuration to the contracted configuration. A device (e.g., the device 188) can be positionable in the device space 752. When the device is positioned in the device space 752 and the shuttle 14 has the contracted configuration, the shuttle 14 can be moveable into the device 188 via the loader control 762. The device 188 can be, for example, a tissue piercer. The device 188 can be, for example, a suture device. The device 188 can be, for example, a tissue piercer and a suture device. The device 188 can be for example, a suture device that can pierce tissue, for example, with the shuttle 14. The device 188 can be for example, a suture device that can pass suture through tissue, for example, with the shuttle 14.

The loader 750 can be used to load the device 188 with the shuttle 14 and/or with the suture 70.

Any systems, devices, features, and/or methods disclosed, illustrated, and/or contemplated in U.S. application Ser. No. 14/255,945 filed Apr. 17, 2014 (published as US 2014/0316443), in International Application No. PCT/US2019/025203 filed Apr. 1, 2019 (published as WO 2019/191768), in U.S. application Ser. No. 16/733,740 filed Jan. 3, 2020 (published as US Publication No. 2021/0204934), in International Application No. PCT/US2020/060888 filed Nov. 17, 2020 (published as WO 2021/137963), in U.S. application Ser. No. 16/734,406 filed Jan. 6, 2020 (published as US Publication No. 2021/0204935), in PCT/US2020/060914 filed Nov. 17, 2020 (published as WO 2021/141675), and/or in U.S. application Ser. No. 17/446,259 filed Aug. 27, 2021 titled METHOD AND APPARATUS FOR PASSING SUTURE, each of which is herein incorporated by reference in its entirety for all purposes—can be used with the loader 750 for any purpose (e.g., to load any of the systems, devices, and/or features in any of these applications with a suture (e.g., the suture 70) and/or with a shuttle (e.g., the shuttle 14) and/or to unload any of the systems, devices, and/or features in any of these applications with a suture (e.g., the suture 70) and/or with a shuttle (e.g., the shuttle 14)) and/or can be combined with any of the systems, devices, features, and/or methods disclosed, illustrated, and/or contemplated herein for any purpose.

It is apparent to one skilled in the art that various changes and modifications can be made to this disclosure, and equivalents employed, without departing from the spirit and scope of the invention. Elements shown with any variation are exemplary for the specific variation and can be used on other variations within this disclosure. Any elements described herein as singular can be pluralized (i.e., anything described as “one” can be more than one). Any species element of a genus element can have the characteristics or elements of any other species element of that genus. The words “may” and “can” are interchangeable (e.g., “may” can be replaced with “can” and “can” can be replaced with “may”). The above-described configurations, elements or complete assemblies and methods and their elements for carrying out the invention, and variations of aspects of the invention can be combined and modified with each other in any combination. Any phrase involving an “A and/or B” construction or similar construction can mean (1) A alone, (2) B alone, (3) A and B together. Any range disclosed can include any subrange of the range disclosed, for example, a range of 1-10 units can include 2-10 units, 8-10 units, or any other subrange.

We claim:

1. A loader comprising:
a body having a device space;
a loader control; and
a shuttle,
wherein the shuttle is moveable from a shuttle first position to a shuttle second position via the loader control,
wherein a device is positionable in the device space,
wherein when the device is positioned in the device space, the shuttle is moveable from the shuttle first position to the shuttle second position, and
wherein the shuttle is more contracted when the shuttle is in the shuttle second position than when the shuttle is in the shuttle first position.

2. The loader of claim 1, wherein when the shuttle is in the shuttle first position, the shuttle has a non-contracted configuration, and wherein when the shuttle is in the shuttle second position, the shuttle has a contracted configuration.

3. The loader of claim 2, wherein the shuttle is wider when the shuttle is in the non-contracted configuration than when the shuttle is in the contracted configuration.

4. The loader of claim 3, wherein when the shuttle is in the shuttle first position, the shuttle is outside the device, and wherein when the shuttle is in the shuttle second position, the shuttle is inside the device.

5. The loader of claim 2, wherein a first end of the shuttle is wider when the shuttle is in the non-contracted configuration than when the shuttle is in the contracted configuration, and/or wherein a second end of the shuttle is wider when the shuttle is in the non-contracted configuration than when the shuttle is in the contracted configuration.

6. The loader of claim 5, wherein when the shuttle is in the shuttle first position, the first end of the shuttle and the second end of the shuttle are outside the device, and wherein when the shuttle is in the shuttle second position, the first end of the shuttle is inside the device and the second end of the shuttle is outside the device.

7. The loader of claim 5, wherein when the shuttle is in the shuttle first position, the first end of the shuttle and the second end of the shuttle are outside the device, and wherein when the shuttle is in the shuttle second position, the first end of the shuttle and the second end of the shuttle are inside the device.

8. The loader of claim 2, wherein when the shuttle has the non-contracted configuration, a first longitudinal end of the shuttle has a non-contracted configuration and a second longitudinal end of the shuttle has a non-contracted configuration.

9. The loader of claim 2, wherein when the shuttle has the contracted configuration, a first longitudinal end of the shuttle has a contracted configuration and a second longitudinal end of the shuttle has a contracted configuration, and/or wherein when the shuttle has the contracted configuration, the first longitudinal end of the shuttle is more contracted than the second longitudinal end of the shuttle.

10. The loader of claim 9, wherein when the shuttle has the contracted configuration, the first longitudinal end of the shuttle is inside the device and the second longitudinal end of the shuttle is outside the device, or wherein when the shuttle has the contracted configuration, the first longitudinal end of the shuttle and the second longitudinal end of the shuttle are inside the device.

11. The loader of claim 2, wherein when the shuttle has the non-contracted configuration, a width of a contractible and expandable portion of the first longitudinal end of the shuttle is the same as a width of a contractible and expandable portion of the second longitudinal end of the shuttle.

12. The loader of claim 11, wherein when the shuttle has the non-contracted configuration, the width of the contractible and expandable portion of the first longitudinal end of the shuttle and the width of the contractible and expandable portion of the second longitudinal end of the shuttle are measured along an axis perpendicular to a center longitudinal axis of the shuttle.

13. The loader of claim 2, wherein when the shuttle has the non-contracted configuration, a width of the widest portion of a contractible and expandable section of the first longitudinal end of the shuttle is less than or greater than a width of the widest portion of a contractible and expandable section of the second longitudinal end of the shuttle.

14. The loader of claim 2, wherein when the shuttle has the contracted configuration, a width of the widest portion of a contractible and expandable section of the first longitudinal end of the shuttle is the same as a width of the widest portion of a contractible and expandable section of the second longitudinal end of the shuttle.

15. The loader of claim 2, wherein when the shuttle has the contracted configuration, a width of the first longitudinal end of the shuttle is the less than a width of the second longitudinal end of the shuttle, or wherein when the shuttle has the contracted configuration, a width of the widest portion of a contractible and expandable section of the first longitudinal end of the shuttle is less than a width of the widest portion of a contractible and expandable section of the second longitudinal end of the shuttle.

16. The loader of claim 15, wherein when the shuttle has the non-contracted configuration, the width of the first longitudinal end of the shuttle and the width of the second longitudinal end of the shuttle are measured along an axis perpendicular to a center longitudinal axis of the shuttle.

17. The loader of claim 1, further comprising a deflector, wherein the shuttle is engageable with the deflector, and wherein when the shuttle is engaged with the deflector, the shuttle has the contracted configuration.

18. The loader of claim 1, further comprising a suture, wherein the shuttle is connectable to the loader control via the suture, and wherein the suture is under more tension when the shuttle is in the shuttle second position than when the shuttle is in the shuttle first position.

19. A loader comprising:
a body having a device space;
a loader control;
a deflector; and
a shuttle,
wherein the shuttle is contractible and expandable,
wherein the shuttle is contractible via the deflector,
wherein when the shuttle is in contact with the deflector, the shuttle has a contracted configuration,
wherein a device is positionable in the device space, and wherein when the device is positioned in the device space and the shuttle has the contracted configuration, the shuttle is moveable into the device via the loader control.

20. A method of loading and/or unloading a device, the method comprising:
loading a shuttle and/or a suture into the device, and/or
unloading the shuttle and/or the suture from the device,
wherein loading the shuttle comprises contracting and/or expanding the shuttle, and
wherein unloading the shuttle comprises contracting and/or expanding the shuttle.
